(19) United States
(12) Patent Application Publication
(10) Pub. No.: US 2011/0177970 A1
(43) Pub. Date: Jul. 21, 2011

(54) METHODS FOR PREDICTING OR MONITORING WHETHER A PATIENT AFFECTED BY A CANCER IS RESPONSIVE TO A TREATMENT WITH A MOLECULE OF THE TAXOID FAMILY

(75) Inventors: Anne Chauchereau, Fontenay-Aux-Roses (FR); Nader Al Nakouzi, Rue Beraniyeh (LB)

(73) Assignees: Inserm (Institut National de la Sante et de la Recherche Medicale), Paris (FR); Institut Gustave Roussy, Villejuif (FR)

(21) Appl. No.: 13/121,975

(22) PCT Filed: Oct. 2, 2009
(86) PCT No.: PCT/EP09/62851

§ 371 Date: Mar. 31, 2011

(30) Foreign Application Priority Data
Oct. 2, 2008 (EP) 08305634.1

(51) Int. Cl. C40B 30/00; C40B 40/06; G01N 33/68; C12Q 1/68

(52) U.S. Cl. 506/9; 506/16; 435/7.1; 435/6.1

(57) ABSTRACT

The present invention concerns in vitro methods for predicting or monitoring whether a patient affected by a cancer is responsive to a treatment with a molecule of the taxoid family based on a resistance expression signature, kits for performing the methods, and methods for screening or identifying a compound suitable for improving the treatment of a cancer with a molecule of the taxoid family or for reducing the resistance development during the treatment of a cancer with the molecule of the taxoid family.

### FIELD OF THE INVENTION

The present invention relates to method for predicting the response to a treatment with a molecule of the taxoid family, kits and method for screening compounds useful for improve the treatment with the molecule.

### BACKGROUND OF THE INVENTION

Prostate cancer became, based on frequency and in Western countries, the first cancer in men, behind the lung cancer. This disease is the second cause of cancer death in men. Since 2005, more than 60,000 men are touched by prostate cancer (PCa) each year and 10,000 men died of this disease. The efficiency of docetaxel chemotherapy (Taxotere®) in prostate cancer (CaP) has been demonstrated for the first time in 2004 in two clinical trials, i.e. TAX 327 and SWOG 99-16, with an increase in survival. Accordingly, docetaxel became today a treatment of choice of metastatic hormone-refractory prostate cancers and phase III clinical trials are ongoing to assess its efficacy for the treatment of high-risk localized prostate cancer. Taxotere® is currently approved in 5 different cancer types in Europe and the US: Prostate cancer, breast cancer, lung cancer, gastric cancer and head and neck cancer. However, in spite of the survival benefit provided by this molecule, docetaxel has a great toxicity and almost half of the patients treated with docetaxel develop a resistance to the chemotherapy either from the beginning, or in a secondary way. Moreover, docetaxel is not effective on all the types of cancer. For instance, in case of breast cancer, only 30 to 50% of the metastatic tumours respond to docetaxel. Resistance to taxanes is common and there is an increasing need to try and identify those patients who will respond to treatment.

A genomic analysis was performed with two cell lines (PC3 and DU145) resistant to a docetaxel dose of 11 nM (Patterson et al, Oncogene, 2006, 25: 6113-6122). The article discloses an expression signature of 30 genes. The authors also demonstrated the effect of STAT1 and Clusterin in an in vitro model for the docetaxel resistance. However, the validation of the expression of these two genes in the docetaxel-resistance has not been performed on tumours. The authors further demonstrated that resveratrol leads to a decreased expression of clusterin in docetaxel resistant cells and, then to an increase of apoptosis (Sallman et al, Mol. Can. Ther., 2007, 6: 2938-2947). Other groups used docetaxel resistance cell lines (PC3-R) in their research (Lo Nigro et al, BJU Int., 2008, 102: 622-7). Some other groups used prostate cancer cell lines treated during a short period (24-72 h) with docetaxel for studying the role of genes in the docetaxel response.

In addition, a patent application WO 2006/062811 concerns a method for measuring resistance or sensitivity to docetaxel.

Therefore, there is still a strong need of a diagnostic method for predicting responsiveness to docetaxel and avoiding useless treatments. Indeed, before the initiation of the treatment, it is currently impossible to identify the patients who will respond to or who will have a resistance to docetaxel.

### SUMMARY OF THE INVENTION

The present invention provides an expression signature specific of the docetaxel resistance in human prostate cancer. Based on this signature, the present invention provides a method for predicting or monitoring whether a patient affected by a cancer is responsive to a treatment with a molecule of the taxoid family.

Accordingly, the present invention concerns an in vitro method for predicting or monitoring whether a patient affected by a cancer is responsive to a treatment with a molecule of the taxoid family, wherein the method comprises: 1) providing a biological sample from said subject; 2) determining in the biological sample the expression level of at least 5 genes selected from the group consisting of the genes listed in Tables 1, 1 bis, 2 and 2 bis, thereby predicting or monitoring whether a patient affected by a cancer is responsive to a treatment with a molecule of the taxoid family. Preferably, the at least 5 genes are selected from the group consisting of the genes listed in Tables 1 and 2. More preferably, the at least 5 genes are selected from the group consisting of RPIB9, CXCL2, AL137761, TFPI2, TNF, ABCB1, PURG, MCTP1, OAS3, GAS1, BIRC3, BQ186674, UBXD3, SFRP1, PLEKHH2, GNG11, CDH16, AKR1C1, MGC42367, AQP1, RAFTLIN, FAM111A, ADAMTS1, FHOD3, DUSP23, ITGB8, IL15, IGFBP3, PHLDA1, GPC3, CACNG6, AKR1C3, C1orf88, CDKN1C, THC2753543, NTN4, MID1, CSPG2, AL133090, ST6GAL1, TMEFF1, FBXL16, CART1, C9orf150, HDAC9, GLS, CNTNAP3, PDE4B, DKFZp586I1420, ZNRF2, SP5, LAMA2, CD55, MANEAL, AK026140, KIAA1505, DEPDC6, PPP2R2C, ARHGDIB, RAI2, TXNRD3, ABCB2, RASSF8, CR622072, LITAF, IGF2, LOC389722, ANKRD18A, GRB10, AY336981, SLP1, COL16A1, GRAMD3, FAM107B, LOC440934, VCX, LAMB3, WNT5A, JAG1, NRL, AGT, TMSB4X, CCPG1, ADRA2C, TEX15, SEMA3B, NFKBIZ, AK096677, PTPRM, NQO1, AK022020, MGAT4A, LOC63920, AL390181, AK123483, FAM80A, PSCDBP, CKB, SLC7A8, PDK1, GATA2, PDLIM5, FLJ10159, PTGES, DNAJC15, NPAS1, THC2668815, TFDP2, PFKFB4, ENC1, NRP2, MFHAS1, AK024680, AL137342, D4S234E, LCP1, A_{—}32_P95067, THC2038567, BDNF, AW205591, AKAP12, NMNAT2, SLC12A3, SLC22A2, ANKRD37, LIN7A, PHEX, C1QL1, EPAS1, KCNC4, FGFBP1, LZTS1, SYTL3, HSHPX5, MGST1, THC2050576, SLC3A1, UGT8, SUNC1, DUSP13, AUTS2, PLAC8, MSX2, SMAD9, TTN, LRRN6C, MEIS2, DHRS3, OLR1, MOXD1, DCAMKL1, C12orf59, SALL1, FZD8, FLJ39502, PROS1, MYB, SLC16A10, GJA7, GAL, PLXNA2, PDE1A, AW467174, PLAT, CXCR4, AK3L1, SMPDL3A, KIAA0960, LHFP, CPM, A_{—}24_P345290, PNOC, GALNT14, TM4SF1, ZAR1, RXFP1, CGNL1, AK094972, SOX9, SLC39A8, TMEM47, SLC10A4, EDG7, ITGA2, SLC1A3, PLCXD3, BF514799, SLC16A12, THC2182743, C4orf18, ANKRD38, and hSHISA3. Optionally, the method comprises determining the expression level of at least 10, 15, 20, 25, 30, 40, 50, 60, 70, 80, 90, 100, 150, 200 or 300 genes from those listed in Tables 1, 1bis, 2 and 2bis, preferably in Tables 1 and 2. Preferably, the cancer is selected from the group consisting of the breast cancer, the lung cancer, the prostate cancer, the gastric cancer and the head and neck cancer. More preferably the cancer is the prostate cancer. Preferably, the expression level is compared to a reference expression level, for instance the expression level of the genes in cell-lines or patients sensitive to the treatment by the molecule of the taxoid family. In particular, the over-expression of genes from Tables 1 and 1 bis, preferably from Table 1, and/or the under-expression of genes from Tables 2 and 2 bis, preferably from Table 2, are indicative of a resistance to the treatment by the molecule of the taxoid family. More preferably, the over-expression of genes selected from the group consisting of RPIB9, CXCL2, AL137761, TFPI2, TNF, ABCB1, PURG, MCTP1, OAS3, GAS1, BIRC3, BQ186674, UBXD3, SFRP1, PLEKHH2, GNG11, CDH16, AKR1C1, MGC42367, AQP1, RAFTLIN, FAM111A, ADAMTS1, FHOD3, DUSP23, ITGB8, IL15, IGFBP3, PHLDA1, GPC3, CACNG6, AKR1C3, C1orf88, CDKN1C, THC2753543, NTN4, MID1, CSPG2, AL133090, ST6GAL1, TMEFF1, FBXL16, CART1, C9orf150, HDAC9, GLS, CNTNAP3, PDE4B, DKFZp586I1420, ZNRF2, SP5, LAMA2, CD55, MANEAL, AK026140, KIAA1505, DEPDC6, PPP2R2C, ARHGDIB, RAI2, TXNRD3, ABCB2, RASSF8, CR622072, LITAF, IGF2, LOC389722, ANKRD18A, GRB10, AY336981, SLPI, COL16A1, GRAMD3, FAM107B, LOC440934, VCX, LAMB3, WNT5A, JAG1, NRL, AGT, TMSB4X, CCPG1, ADRA2C, TEX15, SEMA3B, NFKBIZ, AK096677, PTPRM, NQO1, AK022020, MGAT4A, LOC63920, and AL390181 and/or the under-expression of genes selected from the group consisting of AK123483, FAM80A, PSCDBP, CKB, SLC7A8, PDK1, GATA2, PDLIM5, FLJ10159, PTGES, DNAJC15, NPAS1, THC2668815, TFDP2, PFKFB4, ENC1, NRP2, MFHAS1, AK024680, AL137342, D4S234E, LCP1, A_{—}32_P95067, THC2038567, BDNF, AW205591, AKAP12, NMNAT2, SLC12A3, SLC22A2, ANKRD37, LIN7A, PHEX, C1QL1, EPAS1, KCNC4, FGFBP1, LZTS1, SYTL3, HSHPX5, MGST1, THC2050576, SLC3A1, UGT8, SUNC1, DUSP13, AUTS2, PLAC8, MSX2, SMAD9, TTN, LRRN6C, MEIS2, DHRS3, OLR1, MOXD1, DCAMKL1, C12orf59, SALL1, FZD8, FLJ39502, PROS1, MYB, SLC16A10, GJA7, GAL, PLXNA2, PDE1A, AW467174, PLAT, CXCR4, AK3L1, SMPDL3A, KIAA0960, LHFP, CPM, A_{—}24_P345290, PNOC, GALNT14, TM4SF1, ZAR1, RXFP1, CGNL1, AK094972, SOX9, SLC39A8, TMEM47, SLC10A4, EDG7, ITGA2, SLC1A3, PLCXD3, BF514799, SLC16A12, THC2182743, C4orf18, ANKRD38, and hSHISA3 are indicative of a resistance to the treatment by the molecule of the taxoid family. The expression level of genes can be determined by the quantity of protein or mRNA encoded by said genes. Preferably, the biological sample is a cancer sample.

Preferably, the at least 5 genes are selected from one of the following groups or a combination thereof:

a) RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, WNT2B, GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7, ITGA2, PLCXD3, BF514799, SLC16A12, THC2208430, THC2182743, C4orf18, ANKRD38, CALCRL, and LOC152573, preferably RPIB9, CXCL2, TFPI2, TNF, ABCB1, ADAMTS5, PURG, OAS3, GAS1, BIRC3, MAL, GALNT14, TM4SF1, RXFP1, ATP8A1, SOX9, SLC39A8, EDG7, ITGA2, SLC1A3, CALCRL and LOC152573, more preferably RPIB9, CXCL2, AL137761, TFPI2, TNF, ABCB1, PURG, MCTP1, OAS3, GAS1, BIRC3, BQ186674, UBXD3, GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38 and LOC152573/SHISA3, still more preferably RPIB9, CXCL2, TFPI2, TNF, ABCB1, PURG, OAS3, GAS1, BIRC3, GALNT14, TM4SF1, RXFP1, SOX9, SLC39A8, SLC1A3, EDG7, ITGA2, and LOC152573/SHISA3;

b) RPIB9, TFPI2, ABCB1, BIRC3, WNT2B, SFRP1, FSTL1, AHR, CDKN1C, ABCB2, CYR61, WNT5A, ABCC3, JAG1, STAT1, WNT7B, CASP8, LZTS1, FZD8, GALNT14, RXFP1 and LOC152573;

c) TFPI2, AL137761, RPIB9, PURG, ABCB1, BIRC3, CXCL2, TNF, MCTP1, FAM111A, OAS3, ITGA2, TMEM47, SLC16A12, THC2182743, ANKRD38, SLC1A3, SLC39A8, CXCR4, PDE1A, LOC152573/SHISA3, BF514799, GALNT14 and PLAT;

d) RPIB9, MID1, AQP1, TMSB4X, PDE4B, CDKN1C, ST6GAL1, SUSD4, AGT, CD55, NRL, THC2665111, UGT8, MYB, GALNT14, SMAD9, DNAJC15, and SHISA3;

e) CDH16, AQP1, PLEKHH2, SFRP1, C1orf88, AL133090, CDKN1C, IGF2, CCPG1, KIAA1505, COL16A1, LOC63920, PTGES, BDNF, THC2668815, MFHAS1, LCP1, C12orf59, FGFBP1, EPAS1, SYTL3, LZTS1, OLR1, PDE1A, PLCXD3, ANKRD38, and THC2182743;

f) ABCC3, CD55, COL16A1, DHRS3, FSTL1, GLS, HDL, HIVEP1, LAMA2, LAMB3, LIPG, LITAF, MAL, MFHAS1, NFKBIZ, NRP1, NRP2, OAS3, OLR1, PSCDBP, RFTN1, SCARB1, SEMA3B, SEMA3C, SFRP1, SLC1A3, ST6GAL1, TLR3, TM4SF1 and TNF;

g) ADAMTS1, ADRA2C, AKAP12, CDKN1C, CYR61, FBN1, GAS1, GPC3, IGF2, IGFBP3, JAG1, MGST1, NTN4, PDE1A, PDE4B, PDE4D, PDE4DIP, PDGFB, PHLDA1, PIM1, PPP2R2C, RGS16, SCD, SLC1A1, SMPDL3A, TFPI2 and VCAN;

h) ABCB1, AHR, AHRR, AMPH, BIRC3, CXCL2, CYP1A1, IL1R1, NQO1, PLAT, PLXNA2, SLC16A10, SLC3A1, SLC7A8, SLPI, TAP1, UGT8, UGT2B4, UGT2B7, UGT2B10, UGT2B11 and UGT2B28;

i) AQP1, ARHGDIB, BAMBI, CREB5, CXCR4, EPAS1, FGF2, FGFBP1, GRB10, IL15, MT2A, NUPR1, PDK1, PROS1, PTPN3, RPS6KA2, TFDP2, WNT2B, WNT5A and WNT7B;

j) AGT, ATP8A2, BDNF, EDG6, GAL, GATA2, ITGA2, LRP11, LZTS1, MYB, NCALD, PNOC, PTGES, SRGAP3, TAC3 and TTN;

k) AFF1, ASGR1, BLVRA, CASP8, CD40, KCNH2, NRG1, NRL, PHEX, PLAC8, SMAD7, SMAD9, SOX9, SPG20 and STAT1;

l) TNF, ABCB1, CYP1A1, AHRR, AHR, PP2R2C, ABCC3, NQO1, PIK3C3, UGT2B7, UGT2B11, UGT2B28, UGT2B4, UGT2B10, CHST7, MGST1 and UGT8;

m) Wnt2B, Wnt5A, Wnt7B, SFRP1, FSTL1, Jag1, Cyr61, LOC152573, FZD8 and FOXL2;

n) ADAMRS1, COL16A1, PSCDBP, DHRS3, GAS1, GLS, GPC3, IGF2, IGFBP3, LAMA2, LAMB3, LITAF, MFHAS1, MGST1, NFKBIZ, OAS3, OLR1, PHLDA1, PLAT, PNOC, RAFTLIN, RXFP1, SFRP1,SLC1A3, SLPI, ST6GAL1, TFPI2, TM4SF1, TNF, CSPG2 and WNT5A;

o) ABCB1, ADRA2C, AHR, AKAP12, BIRC3, CD44, CDH16, CDKN1C, CXCL2, EPAS1, HDAC9, MYB, PLXNA2, PTPRM, ROBO3, SLC16A10, SLC3A1, SLC7A8, ABCB2, TFDP2 and TNFSF13;

p) AQP1, GALNT14, ITGA2, ITGB8, NMNAT2, NPAS1, PDLIM5, SEMA3B, SLC12A3, SLC39A8, KIAA0960, TXNRD3, CSPG2 and ZNRF2;

q) CART1, CKB, EBF3, KRT7, LCP1, LRRN6C, THC2182743, MEIS2, NRP2, PROS1, RPIB9, SMPDL3A, UBXD3 and UGT8;

r) AKR1C1, C1QL1, CCPG1, D4S234E, DUSP23, FAM111A, FBXL16, GAL, MGAT4A, MIDI, FAM80A and TMSB4X;

s) PCDH7, LPHN2, CBLN2, FAM19A2, SESN3, NEBL, ST6GAL1, LIN7A, ZMYND12, TCEA3, ADD3, WNT54, TFF1, ACOT9, PCGF5, TUBB6, GBP1, BIRC3, KIF208 and FAM59A;

t) JAK3, ADD3, AKAP9, B3GALT4, BRCA2, CDK6, DEPDC1, GMNN, GULP1, NDUFAF4, PCGF5, SESN3, TUBB6, ZNF91 and WNT5A; and,

u) ACOT9, FUT3, LIN7A, NEBL, PCDH7, ST6GAL1, ASRGL1, BIRC3, BMP7, GBP1, KCND2, KIF20B and NAB1.

In a preferred embodiment, the molecule of the taxoid family is selected from the group consisting of docetaxel, larotaxel, XRP6258, BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321. More preferably, the molecule of the taxoid family is docetaxel.

The present invention also concerns kits and DNA chips suitable for this method. Accordingly, the present invention concerns a kit for predicting or monitoring whether a patient affected by a cancer is responsive to a treatment with a molecule of the taxoid family, wherein the kit comprises detection means selected from the group consisting of a pair of primers, a probe and an antibody specific to at least 5 genes selected from the group consisting of the genes listed in Tables 1, ibis, 2 and 2bis, preferably in Tables 1 and 2, or a DNA chip comprising a solid support which carries nucleic acids that are specific to at least 5 genes selected from the group consisting of the genes listed in Tables 1, ibis, 2 and 2bis, preferably in Tables 1 and 2. Preferably, the at least 5 genes of the kit or DNA chip according to the present invention are selected from one of the following groups or a combination thereof:

a) RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, WNT2B, GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7, ITGA2, PLCXD3, BF514799, SLC16A12, THC2208430, THC2182743, C4orf18, ANKRD38, CALCRL, and LOC152573, preferably RPIB9, CXCL2, TFPI2, TNF, ABCB1, ADAMTS5, PURG, OAS3, GAS1, BIRC3, MAL, GALNT14, TM4SF1, RXFP1, ATP8A1, SOX9, SLC39A8, EDG7, ITGA2, SLC1A3, CALCRL and LOC152573, more preferably RPIB9, CXCL2, AL137761, TFPI2, TNF, ABCB1, PURG, MCTP1, OAS3, GAS1, BIRC3, BQ186674, UBXD3, GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC152573/SHISA3, still more preferably RPIB9, CXCL2, TFPI2, TNF, ABCB1, PURG, OAS3, GAS1, BIRC3, GALNT14, TM4SF1, RXFP1, SOX9, SLC39A8, SLC1A3, EDG7, ITGA2, and LOC152573/SHISA3,

b) RPIB9, TFPI2, ABCB1, BIRC3, WNT2B, SFRP1, FSTL1, AHR, CDKN1C, ABCB2, CYR61, WNT5A, ABCC3, JAG1, STAT1, WNT7B, CASP8, LZTS1, FZD8, GALNT14, RXFP1 and LOC152573;

c) TFPI2, AL137761, RPIB9, PURG, ABCB1, BIRC3, CXCL2, TNF, MCTP1, FAM111A, OAS3, ITGA2, TMEM47, SLC16A12, THC2182743, ANKRD38, SLC1A3, SLC39A8, CXCR4, PDE1A, LOC152573/SHISA3, BF514799, GALNT14 and PLAT;

d) RPIB9, MID1, AQP1, TMSB4X, PDE4B, CDKN1C, ST6GAL1, SUSD4, AGT, CD55, NRL, THC2665111, UGT8, MYB, GALNT14, SMAD9, DNAJC15, and LOC152573/SHISA3;

e) CDH16, AQP1, PLEKHH2, SFRP1, C1orf88, AL133090, CDKN1C, IGF2, CCPG1, KIAA1505, COL16A1, LOC63920, PTGES, BDNF, THC2668815, MFHAS1, LCP1, C12orf59, FGFBP1, EPAS1, SYTL3, LZTS1, OLR1, PDE1A, PLCXD3, ANKRD38, and THC2182743;

f) ABCC3, CD55, COL16A1, DHRS3, FSTL1, GLS, HDL, HIVEP1, LAMA2, LAMB3, LIPG, LITAF, MAL, MFHAS1, NFKBIZ, NRP1, NRP2, OAS3, OLR1, PSCDBP, RFTN1, SCARB1, SEMA3B, SEMA3C, SFRP1, SLC1A3, ST6GAL1, TLR3, TM4SF1 and TNF;

g) ADAMTS1, ADRA2C, AKAP12, CDKN1C, CYR61, FBN1, GAS1, GPC3, IGF2, IGFBP3, JAG1, MGST1, NTN4, PDE1A, PDE4B, PDE4D, PDE4DIP, PDGFB, PHLDA1, PIM1, PPP2R2C, RGS16, SCD, SLC1A1, SMPDL3A, TFPI2 and VCAN;

h) ABCB1, AHR, AHRR, AMPH, BIRC3, CXCL2, CYP1A1, IL1R1, NQO1, PLAT, PLXNA2, SLC16A10, SLC3A1, SLC7A8, SLPI, TAP1, UGT8, UGT2B4, UGT2B7, UGT2B10, UGT2B11 and UGT2B28;

i) AQP1, ARHGDIB, BAMBI, CREB5, CXCR4, EPAS1, FGF2, FGFBP1, GRB10, IL15, MT2A, NUPR1, PDK1, PROS1, PTPN3, RPS6KA2, TFDP2, WNT2B, WNT5A and WNT7B;

j) AGT, ATP8A2, BDNF, EDGE, GAL, GATA2, ITGA2, LRP11, LZTS1, MYB, NCALD, PNOC, PTGES, SRGAP3, TAC3 and TTN;

k) AFF1, ASGR1, BLVRA, CASP8, CD40, KCNH2, NRG1, NRL, PHEX, PLAC8, SMAD7, SMAD9, SOX9, SPG20 and STAT1;

l) TNF, ABCB1, CYP1A1, AHRR, AHR, PP2R2C, ABCC3, NQO1, PIK3C3, UGT2B7, UGT2B11, UGT2B28, UGT2B4, UGT2B10, CHST7, MGST1 and UGT8; and,

m) Wnt2B, Wnt5A, Wnt7B, SFRP1, FSTL1, Jag1, Cyr61, LOC152573, FZD8 and FOXL2;

n) ADAMRS1, COL16A1, PSCDBP, DHRS3, GAS1, GLS, GPC3, IGF2, IGFBP3, LAMA2, LAMB3, LITAF, MFHAS1, MGST1, NFKBIZ, OAS3, OLR1, PHLDA1, PLAT, PNOC, RAFTLIN, RXFP1, SFRP1,SLC1A3, SLPI, ST6GAL1, TFPI2, TM4SF1, TNF, CSPG2 and WNT5A;

o) ABCB1, ADRA2C, AHR, AKAP12, BIRC3, CD44, CDH16, CDKN1C, CXCL2, EPAS1, HDAC9, MYB, PLXNA2, PTPRM, ROBO3, SLC16A10, SLC3A1, SLC7A8, ABCB2, TFDP2 and TNFSF13;

p) AQP1, GALNT14, ITGA2, ITGB8, NMNAT2, NPAS1, PDLIM5, SEMA3B, SLC12A3, SLC39A8, KIAA0960, TXNRD3, CSPG2 and ZNRF2;

q) CART1, CKB, EBF3, KRT7, LCP1, LRRN6C, THC2182743, MEIS2, NRP2, PROS1, RPIB9, SMPDL3A, UBXD3 and UGT8;

r) AKR1C1, C1QL1, CCPG1, D4S234E, DUSP23, FAM111A, FBXL16, GAL, MGAT4A, MID1, FAM80A and TMSB4X;

s) PCDH7, LPHN2, CBLN2, FAM19A2, SESN3, NEBL, ST6GAL1, LIN7A, ZMYND12, TCEA3, ADD3, WNT54, TFF1, ACOT9, PCGF5, TUBB6, GBP1, BIRC3, KIF208 and FAM59A;

t) JAK3, ADD3, AKAP9, B3GALT4, BRCA2, CDK6, DEPDC1, GMNN, GULP1, NDUFAF4, PCGF5, SESN3, TUBB6, ZNF91 and WNT5A; and,

u) ACOT9, FUT3, LIN7A, NEBL, PCDH7, ST6GAL1, ASRGL1, BIRC3, BMP7, GBP1, KCND2, KIF20B and NAB1.

The present invention further concerns methods for screening or identifying a compound suitable for improving the treatment of a cancer with a molecule of the taxoid family or for reducing the resistance development during the treatment of a cancer with a molecule of the taxoid family. In a first embodiment, the method comprises: 1) providing a cell-line with at least 5 genes over-expressed and/or under-expressed respectively selected from the group of over-expressed genes of Tables 1, 1bis and 5-7, preferably in Table 1, and under-expressed genes of Tables 2, 2bis and 5-7, preferably in Table 2; 2) contacting said cell-line with a test compound; 3) determining the expression level of said at least 5 genes; and, 4) selecting the compound which decreases the expression level of over-expressed genes and increases the expression level of under-expressed genes. In a second embodiment, the method comprises: 1) providing a cell-line sensitive to the molecule of the taxoid family; 2) contacting said cell-line with a test compound and the molecule of the taxoid family; 3) determining the expression level of said at least 5 genes selected from the genes listed in Tables 1 and 2; and, 4) selecting the compound which inhibits the appearance of an over-expression and/or an under-expression of at least 5 genes respectively selected from the group of genes of Tables 1 1bis, and over-expressed genes of Tables 5-7, preferably of Table 1 and genes of Tables 2 2bis and under-expressed genes of Tables 5-7, preferably of Table 2. In a third embodiment, the method comprises: 1) providing a cell-line with at least on gene over-expressed and/or under-expressed respectively selected from the group consisting of RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, and WNT2B, preferably RPIB9, CXCL2, AL137761, TFPI2, TNF, ABCB1, PURG, MCTP1, OAS3, GAS1, BIRC3, BQ186674, UBXD3, and more preferably, RPIB9, CXCL2, TFPI2, TNF, ABCB1, PURG, OAS3, GAS1 and BIRC3 for the over-expressed genes, and GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7, ITGA2, PLCXD3, BF514799, SLC16A12, THC2208430, THC2182743, C4orf18, ANKRD38, CALCRL, and LOC152573, preferably GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC152573/SHISA3, and more preferably GALNT14, TM4SF1, RXFP1, SOX9, SLC39A8, SLC1A3, EDG7, ITGA2 and LOC152573/SHISA3 for the under-expressed genes; 2) contacting said cell-line with a test compound; 3) determining the expression level of said at least one gene; and, 4) selecting the compound which decreases the expression level of over-expressed genes and increases the expression level of under-expressed genes. Preferably, the molecule of the taxoid family is selected from the group consisting of docetaxel, larotaxel, XRP6258, BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321. More preferably, the molecule of the taxoid family is docetaxel.

### BRIEF DESCRIPTION OF THE DRAWINGS

FIG. 1: RT PCR validation of the RPIP9 transcript. RPIP9 is the most over-expressed gene of the resistant phenotype. This gene shows a 34 fold change on the micro-array. In quantitative RT PCR, the expression of this gene showed a 450 fold ratio with the probe 1 (Hs00379227_ml) and more than 1000 fold ratio with the probe 2 (Hs00289927_ml). NT: without docetaxel treatment.

FIG. 2: RT PCR validation of the ABC proteins expression. ABCB1 transcript (Mdr-1) codes for P-gp1 protein which is an ATP-dependent membrane transporter responsible of cellular efflux of substances, in particular anti-tumoral drugs. This gene is frequently over-expressed in resistant phenotype. This gene belongs to the 10 most over-expressed genes in the present signature. At the highest docetaxel concentration, this gene showed a 16.22 fold change on the microarray. FIG. 2A: In quantitative RT-PCR (QRT-PCR), the expression of this gene shows a ratio up to 2000×. FIG. 2B: The P-gp1 protein is also found to be over-expressed in a dose-dependent manner in resistant IGR-CaP1 cells at various doses of docetaxel. FIG. 2C: The genes coding for two other proteins of the same family, i.e., ABCB2 and ABCC3, are also over-expressed, with lower fold changes. ABCB2 has a mean fold change of 3.6 on the microarray and a ratio up to 8.3 in QRT-PCR. ABCC3 has a mean fold change of 3.1 on the microarray and a ratio up to 14.3 in QRT-PCR.

FIG. 3: RT PCR validation of BIRC3 and TFPI2 gene expression. These two genes belong to the 15 most over-expressed genes in the present signature with a fold-change of 10.4 and 21.8, respectively. By QRT-PCR, the expression of these genes shows a ratio of up to 36× and 64, respectively.

FIG. 4: RT PCR validation of the expression of STAT1, Clusterin, AHR and CDKN1C genes. FIG. 4A: The gene encoding STAT1 shows a fold-change of 2.47 on the microarray and a fold change up to 5 by RT PCR. The clusterin gene is slightly over-expressed in the resistant cells. FIG. 4B: Over-expression of nuclear proteins AHR and CDKN1C with a fold change on microarray of 5.4 and 5.38 on the microarray, respectively.

FIG. 5: RT PCR validation of under-expressed genes in the signature. GALNT14 gene belongs to the most under-expressed genes in the present signature with a fold-change—10.26 on the microarray. The gene encoding Caspase 8 is also found to be under-expressed by QRT-PCR (Mean Fold change of −4.51 on the microarray).

FIG. 6: Validation of under-expressed LZST1 gene. FIG. 6A: LZST1 gene has been found to be under-expressed by QRT-PCR (Mean Fold change of −4.53 on the microarray). FIG. 6B: Whereas LZST1 protein is present in sensitive cells, it is absent in resistant cells at high docetaxel concentrations.

FIG. 7: RT PCR validation of under-expressed LOC152573 gene. FIG. 7A: the LOC152573 gene encoding the human homolog of SHISA3 is the most under-expressed gene of the present signature (Mean Fold change of −159.4 on the microarray). QRT-PCR analysis confirms this result in docetaxel resistant IGR-CaP1 cells. FIG. 7B: A strong decrease of the hSHISA gene expression has also been observed in LNCaP cells resistant to 2.5 nM of docetaxel and PC3 cells resistant to 0.5 nM of docetaxel.

FIG. 8: RT PCR validation of the expression of Wnt pathway genes belonging to the present signature. FIG. 8A and FIG. 8B: two Taqman primers were used for determining the amount of the two forms of Wnt2B (S1: Taqman Applied Hs00244632_ml; S2: Taqman Applied Hs00257131_ml). FIG. 8C: Genes encoding the other members of the Wnt family. Wnt5a and Wnt7b are over-expressed in a less extent. FIG. 8D: Genes encoding other members of the Wnt pathway. FIG. 8E: The gene encoding the Frizzled 8 receptor (FDZ8) is under-expressed in docetaxel resistant cells.

FIG. 9: Correlation of gene expression between IGR-CaP1 microarray data and quantitative RT-PCR on training test samples (◯) and on independent validation test samples (). For each gene, the mean fold change was calculated from data obtained for each dose of Docetaxel, and comparisons were based on the Log(Ratio). GE: Gene expression microarray.

FIG. 10: Cytotoxic effect of docetaxel in IGR-CaP1 cells and in the 2 derived clones 3A11 and 3B1. Cytotoxicity was assessed by WST1 cell proliferation assay following 72 h culture with increasing concentration of Docetaxel in the parental IGR-CAP1 cells (), in the 3A11 clone (◯) and in the 3B1 clone (□). The data represent the mean of three different experiments, the error bars represent the SEM. Student-tests showed that results on each clone were significantly different from that of the IGR-CaP1 cell line (p<0.001).

### DETAILED DESCRIPTION OF THE INVENTION

The present invention provides the identification of protein coding genes involved in the mechanism of docetaxel resistance in prostate cancer treatment. The inventors prepared in vitro cellular models of docetaxel resistant prostate cancer by selecting cell clones by pharmaceutical pressure from several cellular model of prostate cancer (i.e., LNCap, and IGR-CaP1 cell lines). IGR-CaP1 cell line became resistant to increasing doses of docetaxel (0.5 nM; 5 nM; 12 nM; 25 nM, 50 nM; 100 nM; 200 nM). LNCaP cell line becomes resistant to docetaxel concentrations of 0.5 nM, 2.5 nM, 5 nM and 12 nM. A micro-array genomic analysis was performed by comparing sensitive and resistant IGR-CaP1 cell lines at four docetaxel concentrations (5; 12; 25 and 50 nM) in first step and than, at two additional docetaxel concentrations (100 and 200 nM). This first step analysis led to the identification of 338 genes associated with the resistant phenotype for all the docetaxel concentrations (by 2D clusterization with a P value <10⁻¹⁰, genes with fold change >2). In this signature, 169 genes were over-expressed and 169 genes were under-expressed. By considering the results at the six docetaxel concentrations, the analysis led to the identification of 209 genes associated with the resistant phenotype for all the docetaxel concentrations (by 2D clusterization with a P value <10⁻¹⁰, genes with fold change >2). In this signature, 104 genes were over-expressed and 105 genes were under-expressed. The over-expression of some signature genes was confirmed by quantitative RT-PCR (e.g., genes RPIP9; ABCB1; ABCB2; ABCC3; BIRC3; TFPI2; AHR; STAT1; CDKN1; WNT2B; WNT5A; WNT7B; SFRP1; FSTL1; Jag1; PURG; ADAMTS5; CXCL2; TNF; CYP1A1; NQO1; C4orf18; OAS3; GAS1; PIM1) and/or by Western blot (ABCB1 protein expression). The over-expression of some genes of this signature has also been observed overexpressed by RT PCR in LNCaP cell line resistant to docetaxel concentrations of 0.5 nM and 2.5 nM (e.g., ABCB1 and WNT2B genes). The under-expression of some signature genes was also confirmed by quantitative RT-PCR (e.g., genes GALNT14; LZTS1; LOC152573; FZD8; ITGA2; SLC16A12; SLC39A8; CGNL1; SOX9) and/or by Western blot (LZTS1 protein expression). The under-expression of some genes of this signature has also been observed underexpressed by RT PCR in LNCaP cell line resistant to docetaxel concentrations of 0.5 nM and 2.5 nM and PC3 cell line resistant to a docetaxel concentration of 0.5 nM (e.g., LOC152573/hSHISA3 gene). A micro-array genomic analysis was performed by comparing sensitive and resistant LNCaP cell lines at four docetaxel concentrations (0.5, 2.5, 5 and 12 nM). This analysis led to the identification of 72 genes associated with the resistant phenotype for all the docetaxel concentrations (by 2D clusterization with a P value <10⁻¹⁰, genes with fold change >2). In this signature, 19 genes were over-expressed and 53 genes were under-expressed. In addition, the inventors compared the IGR-CaP1 expression signature to the LNCaP expression signature and defined a set of 18 common genes. Finally, the inventors identified 2 clones 3A11 and 3B1 showing a more resistant profile towards the Docetaxel compared to the parental IGR-CaP1 cell line. By comparing the IGR-CaP1 expression signature to the expression signatures of these two clones, the inventors identified a set of 27 genes.

On this basis, the inventors identified a set of genes whose combined expression profiles allow to distinguish patients between responder and non-responder to a treatment with a molecule of the taxoid family. A “responder” or “responsive” patient refers to a patient who shows or will show a clinically significant recovery when treated in the cancer when treated with a molecule of the taxoid family. In particular, the size of the tumor will no more increase, decrease or the tumor will disappear.

Therefore, the present invention discloses an expression signature useful for in vitro method for predicting whether a patient suffering of a cancer would be responsive to a treatment with a molecule of the taxoid family. The method comprises determining the expression level of genes from the present expression signature (see Tables 1, 1bis, 2, 2bis, 5, 6 and 7, optionally Tables 1 and 2) in a biological sample of said patient. In particular, the method comprises determining the expression level of at least 5 genes of Tables 1, 1bis, 2, 2bis, 5, 6 and 7, optionally of Tables 1 and 2, in a biological sample of said patient. Preferably, the method comprises determining the expression level of at least 6, 7, 8, 9, 10, 15, 20, 25, 30, 40, 50, 60, 70, 80, 90, 100, 150, 200 or 300 genes of Tables 1 and 2. Alternatively, the method comprises determining the expression level of 5 to 338 genes of Tables 1, 1bis, 2 and 2bis, optionally Tables 1 and 2, optionally of 7 to 330, 8 to 300, 9 to 250, 10 to 325, 15 to 300, 20 to 250, 30 to 200, 40 to 150, 50 to 100, 60 to 90 or 70 to 80.

By “predicting” or “prediction” is intended herein the likelihood that a patient will respond or not to a molecule of the taxoid family and also the extent of the response. Predictive methods of the invention can be used clinically to make treatment decisions by choosing the most appropriate treatment modalities for any particular patient. Therefore, the present invention also concerns a method for selecting a patient suffering of a cancer for a treatment with a molecule of the taxoid family, comprising determining the expression level of at least 5, 6, 7, 8, 9, 10, 15, 20, 25, 30, 40, 50, 60, 70, 80, 90, 100, 150, 200 or 300 genes of Tables 1, 1bis, 2 and 2bis, optionally Tables 1 and 2, in a biological sample of said patient and selecting the patient predicted to be responsive to a treatment with a molecule of the taxoid family.

In a first embodiment, the genes are selected from Tables 1 and 2 on the criteria of “fold change”. Accordingly, the genes with the greatest fold change (in absolute value) are chosen. For instance, the genes associated with a fold change greater (in absolute value) than 2, preferably than 3, 4, 5, 6, 7, 8, 9 or 10, are selected. In a particular embodiment, the genes are selected from the group consisting of RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, WNT2B, GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7, ITGA2, PLCXD3, BF514799, SLC16A12, THC2208430, THC2182743, C4orf18, ANKRD38, CALCRL, and LOC152573, preferably from the group consisting of RPIB9, CXCL2, TFPI2, TNF, ABCB1, ADAMTS5, PURG, OAS3, GAS1, BIRC3, MAL, GALNT14, TM4SF1, RXFP1, ATP8A1, SOX9, SLC39A8, EDG7, ITGA2, SLC1A3, CALCRL and LOC152573. Alternatively, the genes can be selected among the genes validated by RT-PCR, in particular in the group consisting of RPIB9, TFPI2, ABCB1, BIRC3, WNT2B, SFRP1, FSTL1, AHR, CDKN1C, ABCB2, CYR61, WNT5A, ABCC3, JAG1, STAT1, WNT7B, CASP8, LZTS1, FZD8, GALNT14, RXFP1 and LOC152573.

In a second embodiment, the genes are selected from the 209 genes identified with the six docetaxel concentrations, namely selected from the group consisting of RPIB9, CXCL2, AL137761, TFPI2, TNF, ABCB1, PURG, MCTP1, OAS3, GAS1, BIRC3, BQ186674, UBXD3, SFRP1, PLEKHH2, GNG11, CDH16, AKR1C1, MGC42367, AQP1, RAFTLIN, FAM111A, ADAMTS1, FHOD3, DUSP23, ITGB8, IL15, IGFBP3, PHLDA1, GPC3, CACNG6, AKR1C3, C1orf88, CDKN1C, THC2753543, NTN4, MID1, CSPG2, AL133090, ST6GAL1, TMEFF1, FBXL16, CART1, C9orf150, HDAC9, GLS, CNTNAP3, PDE4B, DKFZp586I1420, ZNRF2, SP5, LAMA2, CD55, MANEAL, AK026140, KIAA1505, DEPDC6, PPP2R2C, ARHGDIB, RAI2, TXNRD3, ABCB2, RASSF8, CR622072, LITAF, IGF2, LOC389722, ANKRD18A, GRB10, AY336981, SLPI, COL16A1, GRAMD3, FAM107B, LOC440934, VCX, LAMB3, WNT5A, JAG1, NRL, AGT, TMSB4X, CCPG1, ADRA2C, TEX15, SEMA3B, NFKBIZ, AK096677, PTPRM, NQO1, AK022020, MGAT4A, LOC63920, AL390181, AK123483, FAM80A, PSCDBP, CKB, SLC7A8, PDK1, GATA2, PDLIM5, FLJ10159, PTGES, DNAJC15, NPAS1, THC2668815, TFDP2, PFKFB4, ENC1, NRP2, MFHAS1, AK024680, AL137342, D4S234E, LCP1, A_{—}32_P95067, THC2038567, BDNF, AW205591, AKAP12, NMNAT2, SLC12A3, SLC22A2, ANKRD37, LIN7A, PHEX, C1QL1, EPAS1, KCNC4, FGFBP1, LZTS1, SYTL3, HSHPX5, MGST1, THC2050576, SLC3A1, UGT8, SUNC1, DUSP13, AUTS2, PLAC8, MSX2, SMAD9, TTN, LRRN6C, MEIS2, DHRS3, OLR1, MOXD1, DCAMKL1, C12orf59, SALL1, FZD8, F1139502, PROS1, MYB, SLC16A10, GJA7, GAL, PLXNA2, PDE1A, AW467174, PLAT, CXCR4, AK3L1, SMPDL3A, KIAA0960, LHFP, CPM, A _{—}24_P345290, PNOC, GALNT14, TM4SF1, ZAR1, RXFP1, CGNL1, AK094972, SOX9, SLC39A8, TMEM47, SLC10A4, EDG7, ITGA2, SLC1A3, PLCXD3, BF514799, SLC16A12, THC2182743, C4orf18, ANKRD38, and LOC152573/SHISA3. By considering the higher fold change, the genes are selected from the group consisting of TFPI2, AL137761, RPIB9, PURG, ABCB1, BIRC3, CXCL2, TNF, MCTP1, FAM111A, OAS3, ITGA2, TMEM47, SLC16A12, THC2182743, ANKRD38, SLC1A3, SLC39A8, CXCR4, PDE1A, LOC152573/SHISA3, BF514799, GALNT14 and PLAT.

In a third embodiment, the genes are selected from the 72 genes associated with the resistant phenotype for all the docetaxel concentrations in resistant LNCaP cell lines at four docetaxel concentrations (0.5, 2.5, 5 and 12 nM). The 72 genes are listed in Table 5. By considering the higher fold change, the genes are selected from the group consisting of PCDH7, LPHN2, CBLN2, FAM19A2, SESN3, NEBL, ST6GAL1, LIN7A, ZMYND12, TCEA3, ADD3, WNT54, TFF1, ACOT9, PCGF5, TUBB6, GBP1, BIRC3, KIF208 and FAM59A.

In a fourth embodiment, the genes are selected from the genes identified by comparing the IGR-CaP1 expression signature to the LNCaP expression signature and listed in Table 6. Namely, the genes are selected from the group consisting of RPIB9, MID1, AQP1, TMSB4X, PDE4B, CDKN1C, ST6GAL1, SUSD4, AGT, CD55, NRL, THC2665111, UGT8, MYB, GALNT14, SMAD9, DNAJC15, and LOC152573/SHISA3.

In a fifth embodiment, the genes are selected from the genes identified by comparing the IGR-CaP1 expression signature to the expression signatures of the 2 clones 3A11 and 3B1 and listed in Table 7. Namely, the genes are selected from the group consisting of CDH16, AQP1, PLEKHH2, SFRP1, C1orf88, AL133090, CDKN1C, IGF2, CCPG1, KIAA1505, COL16A1, LOC63920, PTGES, BDNF, THC2668815, MFHAS1, LCP1, C12orf59, FGFBP1, EPAS1, SYTL3, LZTS1, OLR1, PDE1A, PLCXD3, ANKRD38, and THC2182743

In a sixth embodiment, the genes are selected from Tables 1, 1bis, 2 and 2bis on the criteria of a network, that is to say that the genes are selected in one particular network. Accordingly, the genes can be selected in the group consisting of one of the following networks or a combination thereof comprising:

1) ABCC3, CD55, COL16A1, DHRS3, FSTL1, GLS, HDL, HIVEP1, LAMA2, LAMB3, LIPG, LITAF, MAL, MFHAS1, NFKBIZ, NRP1, NRP2, OAS3, OLR1, PSCDBP, RFTN1, SCARB1, SEMA3B, SEMA3C, SFRP1, SLC1A3, ST6GAL1, TLR3, TM4SF1 and TNF;

2) ADAMTS1, ADRA2C, AKAP12, CDKN1C, CYR61, FBN1, GAS1, GPC3, IGF2, IGFBP3, JAG1, MGST1, NTN4, PDE1A, PDE4B, PDE4D, PDE4DIP, PDGFB, PHLDA1, PIM1, PPP2R2C, RGS16, SCD, SLC1A1, SMPDL3A, TFPI2 and VCAN;

3) ABCB1, AHR, AHRR, AMPH, BIRC3, CXCL2, CYP1A1, IL1R1, NQO1, PLAT, PLXNA2, SLC16A10, SLC3A1, SLC7A8, SLPI, TAP1, UGT8, UGT2B4, UGT2B7, UGT2B10, UGT2B11 and UGT2B28;

4) AQP1, ARHGDIB, BAMBI, CREB5, CXCR4, EPAS1, FGF2, FGFBP1, GRB10, IL15, MT2A, NUPR1, PDK1, PROS1, PTPN3, RPS6KA2, TFDP2, WNT2B, WNT5A and WNT7B;

5) AGT, ATP8A2, BDNF, EDGE, GAL, GATA2, ITGA2, LRP11, LZTS1, MYB, NCALD, PNOC, PTGES, SRGAP3, TAC3 and TTN;

6) AFF1, ASGR1, BLVRA, CASP8, CD40, KCNH2, NRG1, NRL, PHEX, PLAC8, SMAD7, SMAD9, SOX9, SPG20 and STAT1;

7) ADAMRS1, COL16A1, PSCDBP, DHRS3, GAS1, GLS, GPC3, IGF2, IGFBP3, LAMA2, LAMB3, LITAF, MFHAS1, MGST1, NFKBIZ, OAS3, OLR1, PHLDA1, PLAT, PNOC, RAFTLIN, RXFP1, SFRP1,SLC1A3, SLPI, ST6GAL1, TFPI2, TM4SF1, TNF, CSPG2 and WNT5A;

8) ABCB1, ADRA2C, AHR, AKAP12, BIRC3, CD44, CDH16, CDKN1C, CXCL2, EPAS1, HDAC9, MYB, PLXNA2, PTPRM, ROBO3, SLC16A10, SLC3A1, SLC7A8, ABCB2, TFDP2 and TNFSF13;

9) AQP1, GALNT14, ITGA2, ITGB8, NMNAT2, NPAS1, PDLIM5, SEMA3B, SLC12A3, SLC39A8, KIAA0960, TXNRD3, CSPG2 and ZNRF2;

10) CART1, CKB, EBF3, KRT7, LCP1, LRRN6C, THC2182743, MEIS2, NRP2, PROS1, RPIB9, SMPDL3A, UBXD3 and UGT8; and,

11) AKR1C1, C1QL1, CCPG1, D4S234E, DUSP23, FAM111A, FBXL16, GAL, MGAT4A, MID1, FAM80A and TMSB4X.

In a seventh embodiment, the genes are selected from Tables 1 and 2 on the criteria of their belonging to the signaling pathway of xenobiotic metabolism. Accordingly, the genes can be for instance selected from the group consisting of TNF, ABCB1, CYP1A1, AHRR, AHR, PP2R2C, ABCC3, NQO1, PIK3C3, UGT2B7, UGT2B11, UGT2B28, UGT2B4, UGT2B10, CHST7, MGST1 and UGT8. In another embodiment, the genes are selected from Tables 1 and 2 because of their membership to the Wnt pathway. Accordingly, the genes can be for instance selected from the group consisting of Wnt2B, Wnt5A, Wnt7B, SFRP1, FSTL1, Jag1, Cyr61, LOC 152573, FZD8 and FOXL2.

In a eighth embodiment, the genes are selected from Table 5 on the criteria of a network, that is to say that the genes are selected in one particular network. Accordingly, the genes can be selected in the group consisting of one of the following networks or a combination thereof comprising:

1′) JAK3, ADD3, AKAP9, B3GALT4, BRCA2, CDK6, DEPDC1, GMNN, GULP1, NDUFAF4, PCGF5, SESN3, TUBB6, ZNF91 and WNT5A; and,

2′) ACOT9, FUT3, LIN7A, NEBL, PCDH7, ST6GAL1, ASRGL1, BIRC3, BMP7, GBP1, KCND2, KIF20B and NAB1.

Of course, the genes can also be selected from a combination of these particular groups.

The method can comprise the step of comparing the expression levels of the genes determined in the sample to reference or control expression levels. The reference or control expression levels are determined with a sample of cells, preferably cancer cells, which are sensitive to the molecule of the taxoid family. Alternatively, reference or control expression levels are determined with a sample of patients or subjects sensitive to the treatment with the molecule of the taxoid family. Hence, an over-expressed gene herein refers to a gene having an increased expression in comparison to the expression level of this gene in a sensitive cell, and an under-expressed gene herein refers to a gene having a decreased expression in comparison to the expression level of this gene in a sensitive cell. However, the man skilled in art understands that other references can be used. For instance, the invention also contemplates a reference level corresponding to the expression level in a cell resistant to the molecule of the taxoid family.

In particular, when the genes selected from the Tables 1 and 1bis are over-expressed, one can predict that the patient would be resistant to a treatment with a molecule of the taxoid family. On the contrary, when the genes selected from the Tables 1 and 1bis are not over-expressed, one can predict that the patient would be responsive to a treatment with a molecule of the taxoid family. At the opposite, when the genes selected from the Tables 2 and 2bis are under-expressed, one can predict that the patient would be resistant to a treatment with a molecule of the taxoid family. On the contrary, when the genes selected from the Tables 2 and 2bis are not under-expressed, one can predict that the patient would be responsive to a treatment with a molecule of the taxoid family. Alternatively, the genes used to predict the responsiveness may be selected in Table 5.

In addition, the genes can be selected in such a way that they comprise some over-expressed genes and some under-expressed ones. In this embodiment, the selected genes can comprise at least 5, 6, 7, 8, 9, 10, 15, 20, 25, 30, 40, 50, 60, 70, 80, 90, 100, or 150 genes of Tables 1 and 1bis and at least 5, 6, 7, 8, 9, 10, 15, 20, 25, 30, 40, 50, 60, 70, 80, 90, 100, or 150 genes of Tables 2 and 2bis. Alternatively, they can be selected in such a way that they comprise only over-expressed or under-expressed genes. In a preferred embodiment, the genes are selected among the genes having the greatest fold change.

In addition to the genes selected from Tables 1, 1bis, 2 and 2bis, the method can also comprise the determination of the expression level for control genes. The control genes are chosen among the genes known to have a constant expression level, in particular between sensitive and resistant cells to a molecule of the taxoid family. In addition, the expression level of at least one control gene is determined in order to normalize the result. For instance, the control gene can be GAPDH, 18S RNA, beta-actine or lamin.

The molecule of the taxoid family refers to a class of anti-tumoral drugs belonging to the taxane family. It can be selected from the group consisting of paclitaxel, docetaxel and analogs, prodrugs or formulations thereof. In particular, analogs, prodrugs or formulations thereof can be for instance selected in the group consisting of larotaxel (also called XRP9881; Sanofi-Aventis), XRP6258 (Sanofi-Aventis), BMS-184476 (Bristol-Meyer-Squibb), BMS-188797 (Bristol-Meyer-Squibb), BMS-275183 (Bristol-Meyer-Squibb), ortataxel (also called IDN 5109, BAY 59-8862 or SB-T-101131; Bristol-Meyer-Squibb), RPR 109881A (Bristol-Meyer-Squibb), RPR 116258 (Bristol-Meyer-Squibb), NBT-287 (TAPESTRY), PG-paclitaxel (also called CT-2103, PPX, paclitaxel poliglumex, paclitaxel polyglutamate or Xyotax™), ABRAXANE® (also called Nab-Paclitaxel; ABRAXIS BIOSCIENCE), Tesetaxel (also called DJ-927), IDN 5390 (INDENA), Taxoprexin (also called docosahexanoic acid-paclitaxel; PROTARGA), DHA-paclitaxel (also called Taxoprexin®), and MAC-321 (WYETH). Also see the review of Hennenfent & Govindan (2006, Annals of Oncology, 17, 735-749). In a preferred embodiment of the present invention, the molecule of the taxoid family is the docetaxel.

The expression level of the selected genes can be determined by measuring the amounts of RNA, in particular mRNA, DNA, in particular cDNA, or protein using a variety of techniques well-known by the man skilled in art. In a particular embodiment, the under-expression of a gene can be indirectly assessed through the determination of the methylation status of its promoter. Indeed, a methylated promoter is indicative of an expression repression, and therefore of an under-expression. At the opposite, an unmethylated promoter is indicative of a normal expression. The methylation state of a promoter can be assessed by any method known by the one skilled in the art, for instance by the methods disclosed in the following documents: Frommer et al (Proc Natl Acad Sci USA. 1992;89:1827-31) and Boyd et al (Anal Biochem. 2004;326:278-80).

The cancer can be selected from the group consisting of the breast cancer, the lung cancer, the prostate cancer, the gastric cancer and the head and neck cancer. In a preferred embodiment, the cancer is the prostate cancer.

The term “biological sample” means any biological sample derived from a patient, preferably a sample which contains nucleic acids or proteins. Examples of such samples include fluids, tissues, cell samples, organs, biopsies, etc. Most preferred samples are cancer tissue samples, in particular breast, lung, prostate, stomach, ovary or head and neck tumor samples. Blood, plasma, saliva, urine, seminal fluid, etc, may also be used. Cancer cells obtain form blood as circulating tumor cells may also be used. The biological sample may be treated prior to its use, e.g. in order to render nucleic acids or proteins available. Techniques of cell lysis, concentration or dilution of nucleic acids or proteins, are known by the skilled person.

Generally, the expression level as determined is a relative expression level (mRNA or protein).

More preferably, the determination comprises contacting the sample with selective reagents such as probes, primers or ligands, and thereby detecting the presence, or measuring the amount, of proteins or nucleic acids of interest originally in the sample. Contacting may be performed in any suitable device, such as a plate, microtiter dish, test tube, well, glass, column, and so forth. In specific embodiments, the contacting is performed on a substrate coated with the reagent, such as a nucleic acid array or chip or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or proteins of the sample.

In a preferred embodiment, the expression level may be determined by determining the quantity of mRNA.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). For instance, the probes and primers can be selected from the Taqman Applied ones cited in the present application.

The nucleic acid primers or probes used herein may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, et 2006)

Other methods for determining the expression level of said genes include the determination of the quantity of proteins encoded by said genes.

Such methods comprise contacting a biological sample with a binding partner capable of selectively interacting with a marker protein present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

The invention further provides a tool for implementing said methods, e.g. a DNA chip comprising a solid support which carries nucleic acids that are specific to at least 5, 6, 7, 8, 9, 10, 15, 20, 25, 30, 40, 50, 60, 70, 80, 90, 100, 150, 200 or 300 genes selected from the group consisting of the genes listed in Tables 1, 1bis, 2, 2bis, 5-7, preferably in Tables 1 and 2. The DNA chip can further comprise nucleic acids for control gene, for instance a positive and negative control or a nucleic acid for an ubiquitous gene in order to normalize the results. In addition, the present invention also provides a kit for implementing said methods comprising detection means that are specific to at least 5, 6, 7, 8, 9, 10, 15, 20, 25, 30, 40, 50, 60, 70, 80, 90, 100, 150, 200 or 300 genes selected from the group consisting of the genes listed in Tables 1, 1bis, 2, 2bis, 5-7, preferably in Tables 1 and 2. In particular, the detection means can be a pair of primers, a probe or an antibody. The kit can further comprise control reagents and other necessary reagents.

In a particular embodiment, the genes are selected for the tool or kit as above detailed for the methods of the invention. Preferably, the at least 5 genes are selected from the group consisting of RPIB9, CXCL2, AL137761, TFPI2, TNF, ABCB1, PURG, MCTP1, OAS3, GAS1, BIRC3, BQ186674, UBXD3, SFRP1, PLEKHH2, GNG11, CDH16, AKR1C1, MGC42367, AQP1, RAFTLIN, FAM111A, ADAMTS1, FHOD3, DUSP23, ITGB8, IL15, IGFBP3, PHLDA1, GPC3, CACNG6, AKR1C3, C1orf88, CDKN1C, THC2753543, NTN4, MID1, CSPG2, AL133090, ST6GAL1, TMEFF1, FBXL16, CART1, C9orf150, HDAC9, GLS, CNTNAP3, PDE4B, DKFZp586I1420, ZNRF2, SP5, LAMA2, CD55, MANEAL, AK026140, KIAA1505, DEPDC6, PPP2R2C, ARHGDIB, RAI2, TXNRD3, ABCB2, RASSF8, CR622072, LITAF, IGF2, LOC389722, ANKRD18A, GRB10, AY336981, SLPI, COL16A1, GRAMD3, FAM107B, LOC440934, VCX, LAMB3, WNT5A, JAG1, NRL, AGT, TMSB4X, CCPG1, ADRA2C, TEX15, SEMA3B, NFKBIZ, AK096677, PTPRM, NQO1, AK022020, MGAT4A, LOC63920, AL390181, AK123483, FAM80A, PSCDBP, CKB, SLC7A8, PDK1, GATA2, PDLIM5, FLJ10159, PTGES, DNAJC15, NPAS1, THC2668815, TFDP2, PFKFB4, ENC1, NRP2, MFHAS1, AK024680, AL137342, D4S234E, LCP1, A_{—}32_P95067, THC2038567, BDNF, AW205591, AKAP12, NMNAT2, SLC12A3, SLC22A2, ANKRD37, LIN7A, PHEX, C1QL1, EPAS1, KCNC4, FGFBP1, LZTS1, SYTL3, HSHPX5, MGST1, THC2050576, SLC3A1, UGT8, SUNC1, DUSP13, AUTS2, PLAC8, MSX2, SMAD9, TTN, LRRN6C, MEIS2, DHRS3, OLR1, MOXD1, DCAMKL1, C12orf59, SALL1, FZD8, F1139502, PROS1, MYB, SLC16A10, GJA7, GAL, PLXNA2, PDE1A, AW467174, PLAT, CXCR4, AK3L1, SMPDL3A, KIAA0960, LHFP, CPM, A_{—}24_P345290, PNOC, GALNT14, TM4SF1, ZAR1, RXFP1, CGNL1, AK094972, SOX9, SLC39A8, TMEM47, SLC10A4, EDG7, ITGA2, SLC1A3, PLCXD3, BF514799, SLC16A12, THC2182743, C4orf18, ANKRD38, and LOC152573/SHISA3. In another preferred embodiment, the at least 5 genes are selected from one of the following groups or a combination thereof:

a) RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, WNT2B, GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7, ITGA2, PLCXD3, BF514799, SLC16A12, THC2208430, THC2182743, C4orf18, ANKRD38, CALCRL, and LOC152573, preferably RPIB9, CXCL2, TFPI2, TNF, ABCB1, ADAMTS5, PURG, OAS3, GAS1, BIRC3, MAL, GALNT14, TM4SF1, RXFP1, ATP8A1, SOX9, SLC39A8, EDG7, ITGA2, SLC1A3, CALCRL and LOC152573, more preferably RPIB9, CXCL2, AL137761, TFPI2, TNF, ABCB1, PURG, MCTP1, OAS3, GAS1, BIRC3, BQ186674, UBXD3, GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC152573/SHISA3, still more preferably RPIB9, CXCL2, TFPI2, TNF, ABCB1, PURG, OAS3, GAS1, BIRC3, GALNT14, TM4SF1, RXFP1, SOX9, SLC39A8, SLC1A3, EDG7, ITGA2, and LOC152573/SHISA3;

b) RPIB9, TFPI2, ABCB1, BIRC3, WNT2B, SFRP1, FSTL1, AHR, CDKN1C, ABCB2, CYR61, WNT5A, ABCC3, JAG1, STAT1, WNT7B, CASP8, LZTS1, FZD8, GALNT14, RXFP1 and LOC152573;

c) TFPI2, AL137761, RPIB9, PURG, ABCB1, BIRC3, CXCL2, TNF, MCTP1, FAM111A, OAS3, ITGA2, TMEM47, SLC16A12, THC2182743, ANKRD38, SLC1A3, SLC39A8, CXCR4, PDE1A, LOC152573/SHISA3, BF514799, GALNT14 and PLAT;

d) RPIB9, MIDI, AQP1, TMSB4X, PDE4B, CDKN1C, ST6GAL1, SUSD4, AGT, CD55, NRL, THC2665111, UGT8, MYB, GALNT14, SMAD9, DNAJC15, and LOC152573/SHISA3;

e) CDH16, AQP1, PLEKHH2, SFRP1, C1orf88, AL133090, CDKN1C, IGF2, CCPG1, KIAA1505, COL16A1, LOC63920, PTGES, BDNF, THC2668815, MFHAS1, LCP1, C12orf59, FGFBP1, EPAS1, SYTL3, LZTS1, OLR1, PDE1A, PLCXD3, ANKRD38, and THC2182743;

f) ABCC3, CD55, COL16A1, DHRS3, FSTL1, GLS, HDL, HIVEP1, LAMA2, LAMB3, LIPG, LITAF, MAL, MFHAS1, NFKBIZ, NRP1, NRP2, OAS3, OLR1, PSCDBP, RFTN1, SCARB1, SEMA3B, SEMA3C, SFRP1, SLC1A3, ST6GAL1, TLR3, TM4SF1 and TNF;

g) ADAMTS1, ADRA2C, AKAP12, CDKN1C, CYR61, FBN1, GAS1, GPC3, IGF2, IGFBP3, JAG1, MGST1, NTN4, PDE1A, PDE4B, PDE4D, PDE4DIP, PDGFB, PHLDA1, PIM1, PPP2R2C, RGS16, SCD, SLC1A1, SMPDL3A, TFPI2 and VCAN;

h) ABCB1, AHR, AHRR, AMPH, BIRC3, CXCL2, CYP1A1, IL1R1, NQO1, PLAT, PLXNA2, SLC16A10, SLC3A1, SLC7A8, SLPI, TAP1, UGT8, UGT2B4, UGT2B7, UGT2B10, UGT2B11 and UGT2B28;

i) AQP1, ARHGDIB, BAMBI, CREB5, CXCR4, EPAS1, FGF2, FGFBP1, GRB10, IL15, MT2A, NUPR1, PDK1, PROS1, PTPN3, RPS6KA2, TFDP2, WNT2B, WNT5A and WNT7B;

j) AGT, ATP8A2, BDNF, EDG6, GAL, GATA2, ITGA2, LRP11, LZTS1, MYB, NCALD, PNOC, PTGES, SRGAP3, TAC3 and TTN;

k) AFF1, ASGR1, BLVRA, CASP8, CD40, KCNH2, NRG1, NRL, PHEX, PLAC8, SMAD7, SMAD9, SOX9, SPG20 and STAT1;

l) TNF, ABCB1, CYP1A1, AHRR, AHR, PP2R2C, ABCC3, NQO1, PIK3C3, UGT2B7, UGT2B11, UGT2B28, UGT2B4, UGT2B10, CHST7, MGST1 and UGT8; and,

m) Wnt2B, Wnt5A, Wnt7B, SFRP1, FSTL1, Jag1, Cyr61, LOC152573, FZD8 and FOXL2;

n) ADAMRS1, COL16A1, PSCDBP, DHRS3, GAS1, GLS, GPC3, IGF2, IGFBP3, LAMA2, LAMB3, LITAF, MFHAS1, MGST1, NFKBIZ, OAS3, OLR1, PHLDA1, PLAT, PNOC, RAFTLIN, RXFP1, SFRP1,SLC1A3, SLPI, ST6GAL1, TFPI2, TM4SF1, TNF, CSPG2 and WNT5A;

o) ABCB1, ADRA2C, AHR, AKAP12, BIRC3, CD44, CDH16, CDKN1C, CXCL2, EPAS1, HDAC9, MYB, PLXNA2, PTPRM, ROBO3, SLC16A10, SLC3A1, SLC7A8, ABCB2, TFDP2 and TNFSF13;

p) AQP1, GALNT14, ITGA2, ITGB8, NMNAT2, NPAS1, PDLIM5, SEMA3B, SLC12A3, SLC39A8, KIAA0960, TXNRD3, CSPG2 and ZNRF2;

q) CART1, CKB, EBF3, KRT7, LCP1, LRRN6C, THC2182743, MEIS2, NRP2, PROS1, RPIB9, SMPDL3A, UBXD3 and UGT8;

r) AKR1C1, C1QL1, CCPG1, D4S234E, DUSP23, FAM111A, FBXL16, GAL, MGAT4A, MID1, FAM80A and TMSB4X;

s) PCDH7, LPHN2, CBLN2, FAM19A2, SESN3, NEBL, ST6GAL1, LIN7A, ZMYND12, TCEA3, ADD3, WNT54, TFF1, ACOT9, PCGF5, TUBB6, GBP1, BIRC3, KIF208 and FAM59A;

t) JAK3, ADD3, AKAP9, B3GALT4, BRCA2, CDK6, DEPDC1, GMNN, GULP1, NDUFAF4, PCGF5, SESN3, TUBB6, ZNF91 and WNT5A; and,

u) ACOT9, FUT3, LIN7A, NEBL, PCDH7, ST6GAL1, ASRGL1, BIRC3, BMP7, GBP1, KCND2, KIF20B and NAB1.

The present invention also relates to the use of a DNA chip or a kit of the invention for preparing a kit for predicting or monitoring whether a patient affected by a cancer is responsive to a treatment with a molecule of the taxoid family. Preferably, the cancer is selected from the group consisting of the breast cancer, the lung cancer, the prostate cancer, the gastric cancer and the head and neck cancer. More preferably the cancer is the prostate cancer. In a preferred embodiment, the molecule of the taxoid family is selected from the group consisting of docetaxel, larotaxel, XRP6258, BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321. More preferably, the molecule of the taxoid family is docetaxel.

The present invention further concerns methods for screening or identifying a compound suitable for improving the treatment of a cancer with a molecule of the taxoid family or for reducing the resistance development during the treatment of a cancer with a molecule of the taxoid family. In a first embodiment, the method comprises: 1) providing a cell-line with at least 5, 6, 7, 8, 9, 10, 15, 20, 25, 30, 40, 50, 60, 70, 80, 90, 100, 150, 200 or 300 genes over-expressed and/or under-expressed respectively selected from the group of over-expressed genes of Tables 1, 1bis, and 5-7, preferably of Table 1, and under-expressed genes of Tables 2, 2bis, and 5-7, preferably of Table 2; 2) contacting said cell-line with a test compound; 3) determining the expression level of said at least 5, 6, 7, 8, 9, 10, 15, 20, 25, 30, 40, 50, 60, 70, 80, 90, 100, 150, 200 or 300 genes; and, 4) selecting the compound which decreases the expression level of over-expressed genes and increases the expression level of under-expressed genes. In a second embodiment, the method comprises: 1) providing a cell-line sensitive to the molecule of the taxoid family; 2) contacting said cell-line with a test compound and the molecule of the taxoid family; 3) determining the expression level of said at least 5, 6, 7, 8, 9, 10, 15, 20, 25, 30, 40, 50, 60, 70, 80, 90, 100, 150, 200 or 300 genes selected from the genes listed in Tables 1, 1bis, 2, 2bis, 5-7, preferably in Tables 1 and 2; and, 4) selecting the compound which inhibits the appearance of an over-expression and/or an under-expression of at least 5, 6, 7, 8, 9, 10, 15, 20, 25, 30, 40, 50, 60, 70, 80, 90, 100, 150, 200 or 300 genes respectively selected from the group of genes of Tables 1, 1bis, and over-expressed genes of Tables 5-7, preferably of Table 1, and genes of Tables 2, 2bis and under-expressed genes of Tables 5-7, preferably of Table 2. In a third embodiment, the method comprises: 1) providing a cell-line with at least on gene over-expressed and/or under-expressed respectively selected from the group consisting of RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, and WNT2B for the over-expressed genes, preferably RPIB9, CXCL2, AL137761, TFPI2, TNF, ABCB1, PURG, MCTP1, OAS3, GAS1, BIRC3, BQ186674, UBXD3, and more preferably, RPIB9, CXCL2, TFPI2, TNF, ABCB1, PURG, OAS3, GAS1 and BIRC3, and GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7, ITGA2, PLCXD3, BF514799, SLC16A12, THC2208430, THC2182743, C4orf18, ANKRD38, CALCRL, and LOC152573, preferably GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC152573/SHISA3, and more preferably GALNT14, TM4SF1, RXFP1, SOX9, SLC39A8, SLC1A3, EDG7, ITGA2 and LOC152573/SHISA3 for the under-expressed genes; 2) contacting said cell-line with a test compound; 3) determining the expression level of said at least one gene; and, 4) selecting the compound which decreases the expression level of over-expressed genes and increases the expression level of under-expressed genes.

Preferably, the cell-line is a cancer cell-line. In particular, the cancer cell-line is specific of the targeted cancer. For instance, if the prostate cancer is to be treated, then the cell-line is a prostate cancer cell-line.

In a preferred embodiment, the molecule of the taxoid family is selected from the group consisting of docetaxel, larotaxel, XRP6258, BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, and MAC-321. More preferably, the molecule of the taxoid family is docetaxel. Preferably, the cancer is selected from the group consisting of the breast cancer, the lung cancer, the prostate cancer, the gastric cancer and the head and neck cancer. More preferably the cancer is the prostate cancer.

The example illustrates the invention without limiting its scope.

### EXAMPLES

Methods

Cell Culture and Selection of Docetaxel-Resistant Clones

The human androgeno-dependent prostate carcinoma cell line LNCaP was maintained in RPMI medium complemented with 10% FBS and antibiotics. The human androgen-independent IGR-CaP1 cell line recently obtained for a localized prostate cancer was maintained in RPMI medium complemented with 10% FBS and antibiotics. Docetaxel-resistant clones were selected by culturing the cells in docetaxel in a dose-escalation manner. Initial culture was done in 0.5 nM docetaxel. Cellular clones surviving in the presence of 0.5 nM docetaxel were maintained in culture during four passages, and then the concentration of docetaxel in the medium was increased to 2.5 nM and subsequently to 12 nM, 25 nM, 50 nM, 100 nM and 200 nM. The same selection methodology was followed with each increase in docetaxel concentration. Once cells were freely dividing in each dose of docetaxel mediums, they were considered as resistant and labelled IGR-CaP1-R, LNCaP-R and PC3-R. IGR-CaP1-R clones were obtained surviving in medium containing respectively 2.5 nM, 5 nM, 12 nM, 25 nM, 50 nM, 100 nM and 200 nM docetaxel. LNCaP-R clones survived in medium containing 0.5 nM, 2.5 nM, 5 nM and 12 nM docetaxel. All the cell cultures were maintained at 70% confluency and medium was changed every 48 h.

Cell Cycle Analysis

Effects of treatments on the stages of the cell cycle were determined using the PI staining technique. Briefly, parental and docetaxel-resistant IGR-CaP1 cells were grown in flasks at a density of 4×10⁶ cells. After allowing for overnight attachment the cells were treated or not with 12 nM docetaxel. Cells were incubated for 48 hr then collected by trypsinization, making sure to include the floating cells. After washing in PBS the cells were fixed and permeabilized using Fix&Perm kit (InVitrogen) according to the manufacturer protocol. Cells were treated with 20 μg DNAse-free RNAse for 30 min and stained with 100 μg propidium iodide (PI) for 30 min. Then percentage of cells in G1, S, G2, M, and subG1 phases were analyzed with FACS Calibur cytometer (Becton Dickinson).

Total RNA Preparation and Reverse Transcription

Total RNA from parental and docetaxel-resistant IGR-CaP1 cells was isolated using TriReagent (Sigma-Aldrich) and purified with RNeasy Micro Kit (Qiagen) according to manufacturer's protocols. Quality of RNA preparation, based on the RNA Integrity Number (RIN), was assessed using the Agilent RNA 6000 Nano Kit as developed on the Agilent 2100 Bioanalyzer device (Agilent Technologies, Palo Alto, Calif.). All specimens included in this study displayed a RIN of 10. RNA samples were frozen in nuclease-free water (Qiagen).

Oligo Microarray Technology

Parental and resistant-cell line total RNAs were directly compared by using Agilent oligonucleotide dual-color technology, running dye-swap and duplicate experiments. Total RNA from the parental IGR-CaP1 cell line without treatment was used as the RNA reference. Total RNA from IGR-CaP1 cells resistant to treatment with 5 nM, 12 nM, 25 nM and 50 nM of docetaxel respectively, were used as samples. Probe synthesis and labeling were performed by Agilent's Low Fluorescent Low input Linear Amplification Kit. Hybridization was performed on the Agilent 4x44K Human 1A (G4112F) long (60-bp) oligonucleotide microarrays (Agilent Technologies) by using reagents and protocols provided by the manufacturer. Feature extraction software provided by Agilent (Version A.9.5.3.1) was used to quantify the intensity of fluorescent images and to normalize results using the linear and lowess subtraction method. Primary analysis was performed by using Resolver software (version 7.1) (Rosetta Laboratories, Milan) to identify genes differentially expressed between parental and resistant cell lines with a fold change >2 and P value <10⁻¹⁰. For the first microarray analysis in IGR-CaP1 cells, using this procedure for each of the 4 combined experiments, a list of 378 genes was extracted and was considered as a signature of gene potentially implicated in resistance to docetaxel. These genes were sorted out by the mean of the fold change observed respectively for the 4 doses of resistance towards docetaxel. Subtraction of genes that were represented with multiple probes, this analysis led to a signature of 338 genes. For the second microarray analysis in IGR-CaP1 cells, the clusterization of the 6 combined experiments provided a list of 244 genes considered as a signature of gene potentially implicated in resistance to docetaxel. Subtraction of genes that were represented with multiple probes, this analysis led to a signature of 209 genes. The same procedure was applied in the docetaxel-resistant LNCaP cells and provided a list of 72 genes considered as a signature of gene potentially implicated in resistance to docetaxel.

TaqMan Real-Time Quantitative Reverse Transcription-PCR Analysis.

Real-time quantitative RT-PCR was performed using the ABI Prism 7900 Sequence Detection System (Perkin-Elmer Applied Biosystems). The same procedure was applied from the total RNA used in the microarray analysis and for independent RNA samples. One μg of total RNA was reversed transcribed using the GeneAmp RNA PCR Kit according to the manufacturer's recommendations (Applied Biosystems).

Quantitative real-time PCR was performed in a final volume of 25 μl according to the manufacturer's recommendations (Applied Biosystems). PCR primers and probe for the selected target genes were designed by Applied Biosystems and used according to the manufacturer's recommendations. The amount of sample RNA was normalized by the amplification of an endogenous control (18S). The relative quantification of the transcripts was derived by using the standard curve method (Applied Biosystems User Bulletin 2, ABI PRISM 7700 Sequence Detection System). The ratio compared the gene expression obtained into the resistant cells to the one of the parental IGR-CaP1 cell line. The following Taqman probes were used: RPIB9 Hs00379227_m1 and Hs00289927_m1; ABCB1 Hs00184491_m1; ABCB2 Hs00388682_m1; ABCC3 Hs00358656_m1: BIRC3 Hs00154109_m1; TFPI2 Hs00197918_m1; STAT1 Hs00234829_m1; CLU Hs00156548_m1, AHR Hs00907314_m1; CDKN1C Hs00175938_m1; GALNT14 Hs00226180_m1; CASP8 Hs01018151_m1; LZTS1 Hs00232762_m1; LOC152573/SHISA3 Hs01380806_m1; WNT2B Hs00244632_m1 and Hs00257131_m1; WNT5A Hs00998537_m1; WNT7B Hs00536497_m1; SFRP1 Hs00610060_m1; FSTL1 Hs00200053_m1; JAG1 Hs01070032_m1; FDZ8 Hs00259040_s1; ITGA2 Hs00158148_m1; PURG Hs00273723. SLC16A12 Hs01584854_m1; ADAMTS5 Hs00199841_m1; CXCL2 Hs00236966_m1; TNF Hs00174128_m1; SLC39A8 Hs00223357_m1; CYP1A1 Hs00153120_m1; NQO1 Hs00168547_m1; C4orf18 Hs00213275_m1; OAS3 Hs00196324_m1; GAS1 Hs00266715_s1; CGNL1 Hs00262671_m1; SOX9 Hs00165814_m1; PIM1 Hs01065498_m1.

Western Blot Analysis

Parental and resistant cellular clones were cultured in 175 cm2 flask in the presence of the appropriate concentration of docetaxel. Cells were lysed in RIPA buffer to prepare whole cell extracts and denatured in NuPage LDS sample buffer (Invitrogen). Protein concentration of the soluble extracts was determined by using the MicroBCA protein assay (Pierce).

Proteins from 50 μg of whole cell extracts were resolved by electrophoresis on NuPage 4-12% Bis-Tris gels (Invitrogen) and immunoblots were developed using the enhanced chemoluminescence-based detection kit (Pierce). The following antibodies were used: anti-ABCB1 (Mdr-1 D-11) and anti-LZTS1 (FEZ1 C-20) from Santa-Cruz Biotechnology Inc. The equal loading of protein sample was verified with a β-actin-specific antibody (Sigma).

Results

Generation of acquired resistance to Docetaxel in vitro. Prostate cancer IGR-CaP1 cells were used to generate successive docetaxel-resistant cell lines. The addition of docetaxel induced a selection process, whereby a large majority of cells initially underwent cell death until the ability to proliferate was regained. The inventors obtained IGR-CaP1 resistant (IGR-CaP1-R) clones which survived in medium containing respectively 5 nM, 12 nM, 25 nM, 50 nM of docetaxel. Cell cycle analysis was done to show acquired resistance to drug. The resistant cell lines showed cell cycle similar to the parental IGR-CaP1 cells, suggesting that acquired resistance had been gained (not shown).

Genome-wide analysis of IGR-CaP1 docetaxel-resistant lines using microarray. Human genome-wide analysis of gene expression changes was realized in order to stringently identify human genes that might represent the molecular signature of resistance or sensitivity to docetaxel in prostate cancer. Untreated IGR-CaP1 parental cell lines were used as baseline. Hierarchical clustering of combined experiments using a 2-fold change criteria and a P value of <10⁻¹⁰ revealed a total of 338 genes that were up or down-regulated by >2-fold in each of the resistant cell lines. 169 genes were over-expressed (Table 1) and 169 were down-regulated (Table 2) in docetaxel-resistant cells. These genes were sorted out by the mean of the fold change observed respectively for the 4 doses or the 6 doses of resistance towards docetaxel (Table 3 and Table 4). Functional analysis of the resistant cell lines was performed using Ingenuity® Pathways Analysis (IPA). Highly significant functions and canonical pathways were found for resistant cell lines as organ development, cancer, cellular growth and proliferation, cellular movement, cell-to-cell signalling and interaction, or cell death.

Target Verification by Real-Time RT-PCR and Western Blot

To verify the alterations of gene expression at the mRNA level, which appeared on the microarray, the inventors chose representative genes with varying expression profiles for real-time Taqman RT-PCR and Western Blot analysis. The inventors measured gene expression levels in a panel of 33 genes.

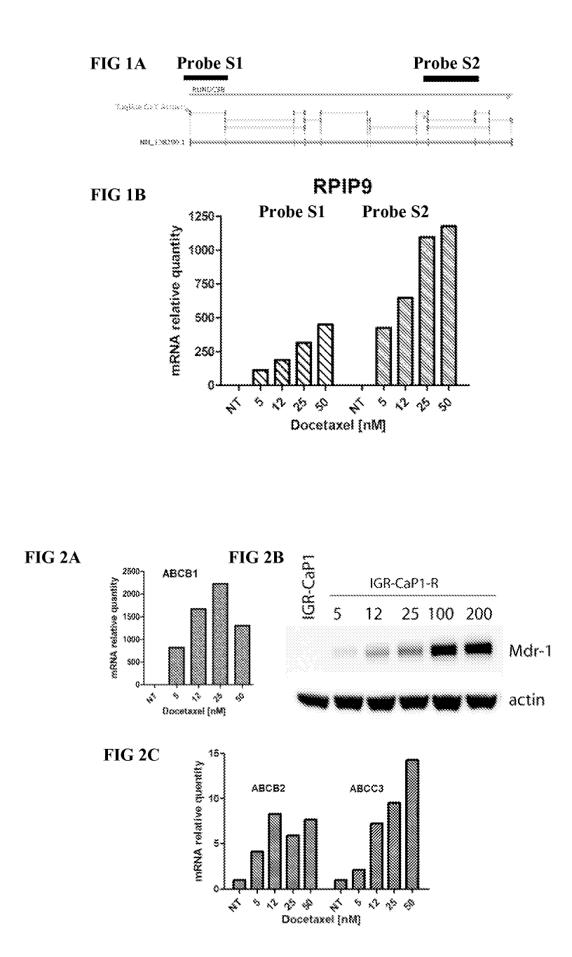

The inventors first measured the expression of the Top gene of the signature, RPIP9/RPIB9/RUNDC3B, encoding Rap2-binding protein 9. Two sets of probes were chosen to measure gene expression of RPIB9 as multiple splice variants were transcribed (FIG. 1A). The two probes showed a high level in gene expression in docetaxel-resistant cells in a dose dependent manner (FIG. 1B). Over expression was more pronounced (more than 1000 fold) with the 3′ probe set, suggesting that long variants containing RUN domain were more expressed. The same results were obtained on an independent set of total RNAs (not shown). The function of RPIP9 protein is not known but RPIP9 gene was shown to be overexpressed in breast carcinoma and correlated with a poor prognosis.

A key mechanism underlying multidrug resistance relates to the overexpression of the ATP-dependent transporter family known as the ATP-binding cassette (ABC) family. One of the most described members of these drug efflux pumps was the P-glycoprotein (P-gp) encoded by the MDR-1 gene. This gene had been frequently found overexpressed in drug-resistant phenotype. The gene ABCB1/MDR1 is one of the most over-expressed genes of the signature. The same alterations of gene expression were observed by real-time RT-PCR analysis, although the fold change in the expression level was much higher (FIG. 2A). The same results were obtained on an independent set of total RNAs (not shown). Western blot analysis showed that expression of the MDR1 gene product was increased in a dose-dependent manner in docetaxel-resistant cells (FIG. 2B). Two other genes encoding members of the ATP-binding cassette family, ABCB2 and ABCC3 were confirmed to be overexpressed in resistant cells, although to a lesser level compared to the ABCB1 gene (FIG. 2C). Interestingly, ABCC3 was recently identified as a mediator of taxane resistance in HER2-amplified breast cancer.

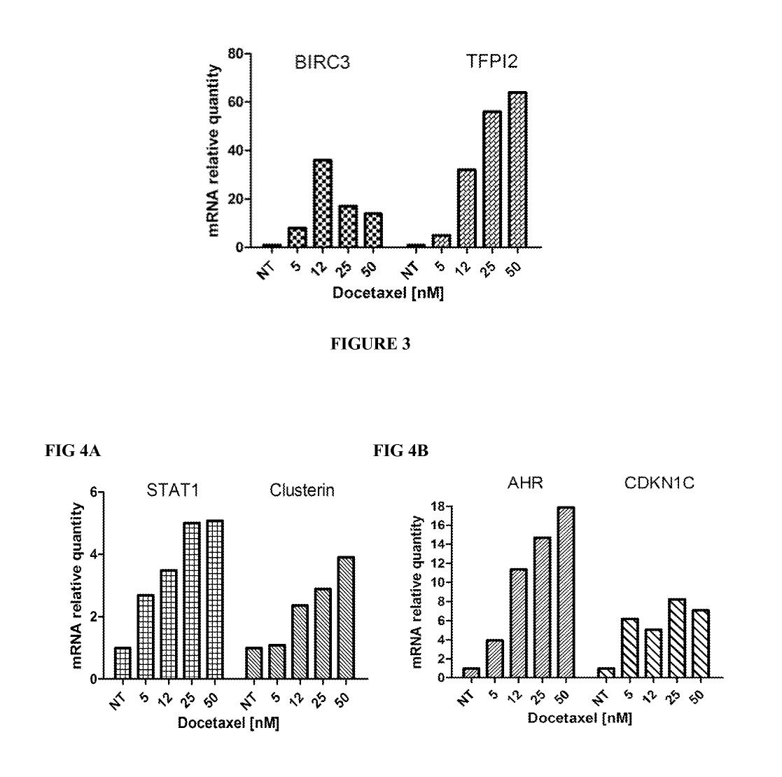

The genes BIRC3 and TFPI2 were found in the Top 15 of over-expressed genes of the signature with a fold-change expression of 10.4 and 21.8 respectively in the resistant cells. BIRC3 encoding baculoviral IAP repeat-containing 3 belongs to a family of proteins that inhibits apoptosis (IAP family). Interestingly, it had been suggested that IAP proteins may have an important contribution to the resistance to the apoptotic effect of cisplatin in prostate cancer. The TFPI2 gene, encoding tissue factor pathway inhibitor 2, is a potent inhibitor of matrix-metalloproteinase. This protein was shown to be most prominently up-regulated in MYCN-amplified neuroblastomas. RT-PCR analysis confirmed that BIRC3 and TFPI2 genes were overexpressed in taxane-resistant cells up to 36 fold and 64 fold respectively (FIG. 3).

As the genes STAT1 and clusterin were showed overexpressed in DU145-DR and PC3-DR docetaxel-resistant cells in the study of Patterson et al., (2006), the inventors verified the expression level of these two genes by RT-PCR in the IGR-CaP 1-R model. STAT1 was also found overexpressed in the present signature with a fold change of 2.47 but Clusterin was not retained in the present microarray analysis. As shown in FIG. 4A, the genes STAT1 and Clusterin were up-regulated in IGR-CaP1-R resistant cells, although to a modest extent. CDKN1C was another gene that had been shown overexpressed in DU145-DR and PC3-DR docetaxel-resistant cells. In the present microarray analysis, the inventors found that CDKN1C gene was also overexpressed with a 5.38 fold change in resistant cells. This result was confirmed by the RT-PCR experiment (FIG. 4B). AHR is a ligand-activated transcription factor that mediates a pleiotropic response to environmental contaminants and that has recently been shown to be implicated in the development of cancers from different anatomical origins. Moreover, AHR had been identified as a putative Wnt/β-Catenin pathway target gene in prostate cancer cells. The AHR gene showed a 5.40 fold overexpression in resistant cells in the present microarray analysis. The inventors confirmed this result and showed a high dose-dependent overexpression in taxane-resistant cells by the RT-PCR approach (FIG. 4B). The high increase in AHR gene expression was confirmed on an independent set of total RNA (not shown).

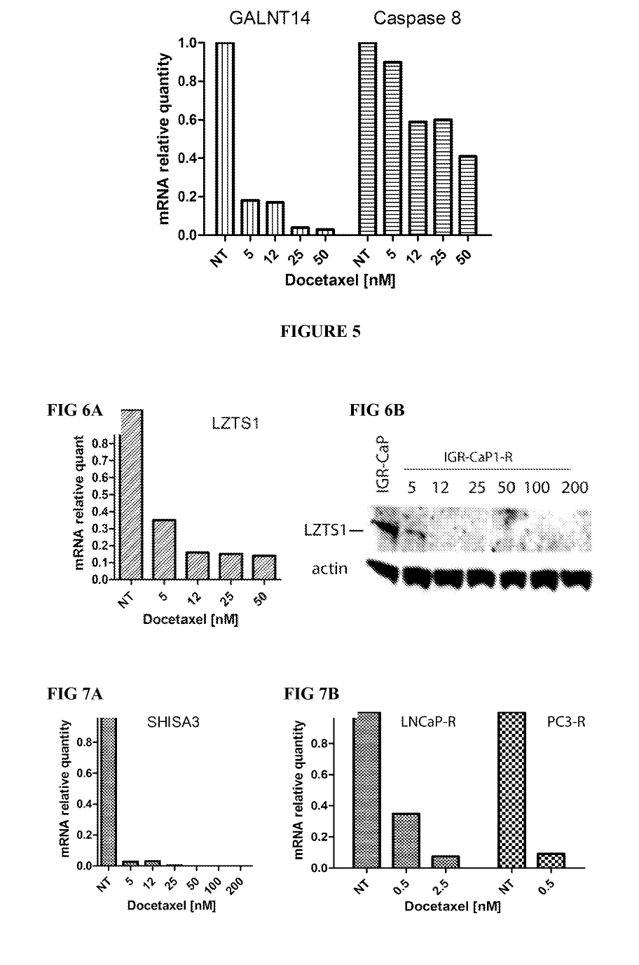

GALNT14 belongs to a large subfamily of glycosyltransferases residing in the Golgi apparatus. GALNT enzymes catalyze the first step in the O-glycosylation of mammalian proteins by transferring N-acetyl-D-galactosamine (GalNAc) to peptide substrates. The GALNT14 gene was one of the top down-regulated genes in the present signature with a fold-change expression of −10.26 in the resistant cells. The dose-dependent down-regulation of the expression of GALNT14 in resistant cells was confirmed by the RT-PCR analysis (FIG. 5). The high decrease in GALNT14 gene expression was confirmed on an independent set of total RNA (not shown). The caspase 8 gene encodes a member of the cysteine-aspartic acid protease (caspase) family. Sequential activation of caspases plays a central role in the execution-phase of cell apoptosis. This gene was founded moderately under-expressed in the RT-PCR analysis (FIG. 5).

LZTS1 encoding leucine zipper, putative tumor suppressor 1 was shown under-expressed in the present signature. The under-expression of this gene was confirmed by RT-PCR analysis (FIG. 6A) and western blot analysis (FIG. 6B) in docetaxel-resistant cells. The same results were obtained on an independent set of total RNAs (not shown). LZTS1 was of particular interest since it has been described as a tumor suppressor gene. The FEZ1/LZTS1 (LZTS1) protein was shown to be frequently downregulated in esophageal, breast, and prostate cancers. LZTS1 is expressed in normal tissues, and its introduction in cancer cells inhibits cell growth and suppresses tumorigenicity. Absence or low expression of LZTS1 was correlated to high tumor grading on lung tumors suggesting that it may serve as a novel prognostic indicator.

The gene LOC152573 encodes the hypothetical protein BC012029 also named hSHISA3. The function of hSHISA3 is not known but by analogy with the mouse homologs, it is supposed to play an essential role in the maturation of presomitic mesoderm cells by individual attenuation of both FGF and WNT signalling. LOC152573/hSHISA3 corresponded to the most under-regulated gene in resistant cells with a fold change of −159.40 in the microarray analysis. The inventors confirmed the high decrease of its expression on independent set of total RNAs of resistant IGR-CaP1-R cells (FIG. 7A) as well as in extracts obtained from LNCaP-R and PC3-R docetaxel-resistant cells (FIG. 7B).

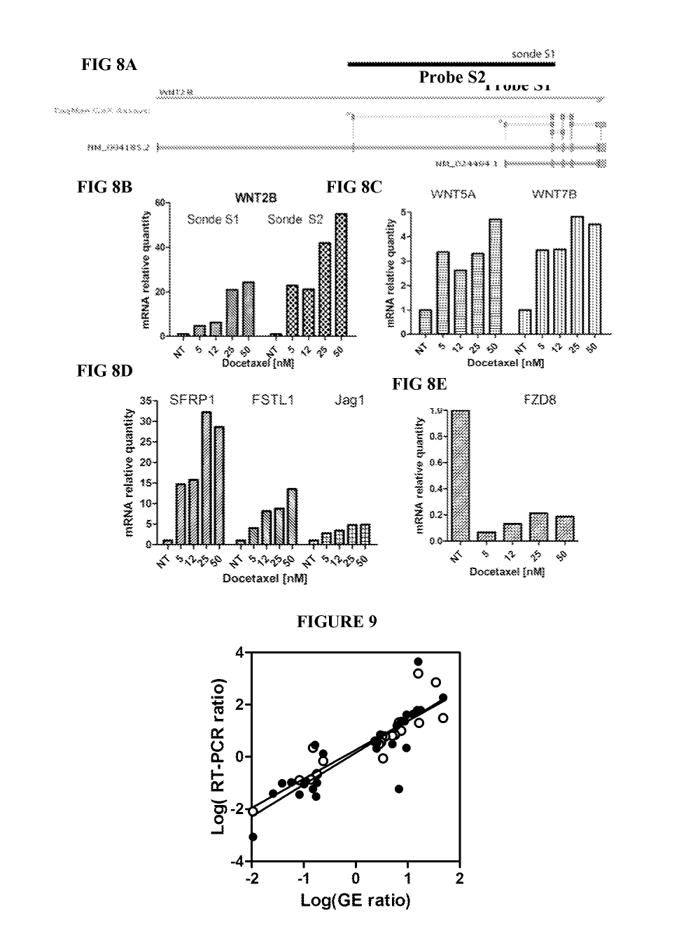

Finally, the inventors checked for the confirmation of the genes implicated in the WNT pathway that were recovered in the microarray analysis. WNT family members function in a variety of developmental processes including regulation of cell growth and differentiation and are characterized by a WNT-core domain. Additionally, WNT signaling has emerged as an important pathway that underlies the initial notion of prostate cancer. Both human cancers and mouse models have confirmed that mutations or altered expression of components of this pathway are associated with prostate tumors. The WNT2B encoding a member of the WNT family of highly conserved, secreted signalling factors, was shown as one of the most over-expressed gene in the signature with a fold change of 9.42 in resistant cells. Two sets of probes were chosen to measure gene expression of WNT2B as this gene produces two alternative transcript variants (FIG. 8A). The two probes showed a high level in gene expression in docetaxel-resistant cells in a dose dependent manner (FIG. 8B). Others members of the WNT gene family, WNT5A and WNT7B genes, were also showed to be overexpressed in drug-resistant cells, although to a lesser extent (FIG. 8C). The gene SFRP1 (Secreted frizzled-related protein 1) acting as soluble modulator of WNT signalling, FSTL1 encoding a protein with similarity to follistatin, and JAG1 encoding the ligand for the receptor Notch 1 were also shown to be up-regulated in the drug-resistant cells, although to different extent (FIG. 8D). On the contrary, the gene FZD8, encoding a member of the frizzled gene family, showed a high decrease of its expression in drug-resistant cells (FIG. 8E).

FIG. 9 showed the good correlation in gene expression between microarray data and QRT-PCR data. When tested on independent samples, the spearman test calculated a high correlation coefficient (rho=0.889). The validation assay was performed on 38 genes belonging to the signature of 338 genes. On these 38 genes, 33 have been validated by QRT-PCR as they showed the same modification in gene expression in microarray and in RT-PCR assay. 28 of these validated genes belong also to the list of 209 genes, 11 of the validated genes belong to the group of the 20 top genes (the upregulated genes TFPI2, RUNDC3B, PURG, ABCB1, BIRC3, CXCL2, TNF, MCTP1, FAM111A, OAS3 and the down-regulated genes ITGA2, TMEM47, SLC16A12, THC2182743, ANKRD38, SLC1A3, SLC39A8, CXCR4, PDE1A and PLAT).

Overall, the results of real-time RT-PCR for these selected genes were in direct agreement with the microarray data. The same alternations of gene expression were observed by real-time RT-PCR analysis, although the fold change in the expression level was not exactly same between these two different analytical methods. Western Blot analyses were also in direct agreement with the microarray data. These results support the findings obtained from the present microarray analysis.

Genome-Wide Analysis of LNCaP Docetaxel-Resistant Lines Using Microarray.

LNCaP cell line became resistant to increasing doses of docetaxel (0.5 nM; 2.5 nM; 5 nM; and 12 nM). The inventors performed a microarray analysis that compare whole genome expression on Docetaxel-resistant LNCaP cells at four docetaxel concentrations (0.5 nM; 2.5 nM; 5 nM; and 12 nM) versus parental LNCaP cells using 44 k micro-array (Agilent). The microarray data showed that 72 genes had either an increase or decrease in expression in all the resistant LNCaP cells, by 2D clusterization with a P value <10⁻¹⁰, genes with fold change >2 (Table 5). In this signature of 72 genes, 19 were overexpressed and 53 genes were under-expressed.

In the hypothesis that genes implicated in the resistance of Docetaxel in prostate cancer should be the same genes in the different prostate cancer cell lines, the inventors consider the genes that were commonly over-expressed or under-expressed in both the IGR-CaP1 and the LNCaP resistant cells. By comparing the list of genes with a fold change >2 in the 6 doses Docetaxel-resistant IGR-CaP1 cell lines (namely 5, 12, 25, 50, 100 and 200 nM of Docetaxel) and in the 2 most Docetaxel-resistant LNCaP cell lines (5 nM and 12 nM of Docetaxel), the inventors extracted a list of 18 common genes that were modified similarly in the two Docetaxel-resistant cellular models (Table 6).

Clonally-Derived IGR-CaP1 Cells

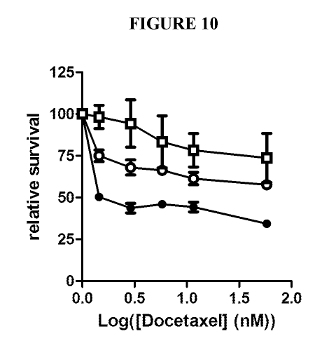

Several clonally-derived cells were obtained from the parental IGR-CaP1 cells by limit dilutions. To evaluate the cytotoxic effect of docetaxel in the parental IGR-Cap1 cell line and in the two of the clonally-derived 3A11 and 3B1 clones, cells were exposed to increasing concentrations of docetaxel (from 1.25 nM to 50 nM) for 72 h, and WST1 cell proliferation assay was performed. Docetaxel decreased cell proliferation in a dose-dependent manner in the three cell lines but the drug affect the proliferation in a significantly lower extent (p<0.001) in the 2 clones 3A11 and 3B1 compared to the parental IGR-CaP1 cells (FIG. 10).

The 2 clones 3A11 and 3B1 showed a more resistant profile towards the Docetaxel compared to the parental IGR-CaP1 cell line, suggesting that they were naturally more resistant to the drug than the parental cells. To test the hypothesis that the expression of gene implicated in the mechanism of resistance could be amplified in these two clones, the inventors compared the whole gene expression profile of the 3A11 and 3B1 clones to that of the IGR-CaP1 cell line by DNA micro-array. This analysis led to the identification of 1203 genes which were differently expressed in the 2 clones (by 2D clusterization with a P value <10⁻¹⁰, genes with fold change >2). 27 genes identified in the resistance signature of 209 genes in the docetaxel-resistant IGR-CaP1 cells were found similarly amplified or downregulated in the naturally-resistant clones 3A11 and 3B1, suggesting that these 27 genes (Table 7) could be implicated in the acquisition of the drug-resistant phenotype and thus could potentially be used as marker of the resistant phenotype.

TABLE 1

| | | | |
|---|---|---|---|
| Primary | | | |
| Sequence Name | Sequence Name(s) | Sequence Description | Accession # |

List of the over-expressed genes (at least two-fold)
in the docetaxel resistant IGR-CaP1 cell-lines.

| | | | |
|---|---|---|---|
| RPIB9 | RPIB9, RPIP9, FLJ30671, MGC26655, | Rap2-binding protein 9 | NM_138290 |
| | RUNDC3B | | |
| CXCL2 | CXCL2, GRO2, GROb, MIP2, MIP2A, | GRO2 oncogene | NM_002089 |
| | SCYB2, MGSA-b, MIP-2a, CINC- | | |
| | 2a, MGSA beta | | |
| AL137761 | AL137761, FAM110C | Homo sapiens mRNA; cDNA DKFZp586L2424 | AL137761 |
| | | (from clone DKFZp586L2424), | CR601458 |
| TFPI2 | TFPI2, PP5, TFPI-2, FLJ21164 | tissue factor pathway inhibitor 2 | NM_006528 |
| THC2051204 | THC2051204 | Q300_MOUSE (Q02722) Protein Q300, partial | |
| | | (17%) [THC2051204] | |
| TNF | TNF, DIF, TNFA, TNFSF2, TNF-alpha | tumor necrosis factor (TNF superfamily, member 2) | NM_000594 |
| ABCB1 | ABCB1, CLCS, MDR1, P- | ATP-binding cassette, sub-family B (MDR/TAP), | NM_000927 |
| | gp, PGY1, ABC20, CD243, GP170 | member 1 | |
| PURG | PURG, PURG-A, PURG- | purine-rich element binding protein G | NM_013357 |
| | B, MGC119274 | | |
| ADAMTS5 | ADAMTS5, ADMP- | a disintegrin-like and metalloprotease (reprolysin | NM_007038 |
| | 2, ADAMTS11, FLJ36738 | type) with thrombospondin type 1 motif, 5 | |
| | | (aggrecanase-2) | |
| MCTP1 | MCTP1, FLJ22344 | Homo sapiens cDNA FLJ34011 fis, clone | AK091330 |
| | | FCBBF2001868, weakly similar to RABPHILIN-3A, | AK025997 |
| SPTLC2L | | Homo sapiens cDNA FLJ90790 fis, clone | AK075271 |
| | | THYRO1001529, weakly similar to SERINE | |
| | | PALMITOYLTRANSFERASE 2 (EC 2, 3, 1, 50), | |
| OAS3 | OAS3, p100, MGC133260 | 2′-5′-oligoadenylate synthetase 3 (100 kD) | NM_006187 |
| GAS1 | GAS1 | growth arrest-specific 1 | NM_002048 |
| BIRC3 | BIRC3, AIP1, API2, MIHC, CIAP2, HAIP1, | baculoviral IAP repeat-containing 3 | NM_001165 |
| | HIAP1, MALT2, RNF49 | | |
| BQ186674 | BQ186674, LOC645638 | UI-E-EJ1-ajr-f-10-0-UI, r1 UI-E-EJ1 Homo sapiens | BQ186674 |
| | | cDNA clone UI-E-EJ1-ajr-f-10-0-UI 5′, | XR_040455 |
| MAL | MAL | mal, T-cell differentiation protein | NM_002371 |
| UBXD3 | UBXD3, FLJ25429 | Homo sapiens UBX domain containing 3 | NM_152376 |
| | | (UBXD3), mRNA [NM_152376] | |
| WNT2B | WNT2B, WNT13, XWNT2 | wingless-type MMTV integration site family, | NM_024494 |
| | | member 2B | |
| BM716045 | BM716045 | UI-E-EJ0-aht-l-14-0-UI, r1 UI-E-EJ0 Homo sapiens | BM716045 |
| | | cDNA clone UI-E-EJ0-aht-l-14-0-UI 5′, | |
| SFRP1 | SFRP1, FRP, FRP1, FrzA, FRP-1, | secreted frizzled-related protein 1 | NM_003012 |
| | SARP2 | | |
| PLEKHH2 | PLEKHH2, KIAA2028, PLEKHH1L | Homo sapiens pleckstrin homology domain | NM_172069 |
| | | containing, family H (with MyTH4 domain) | |
| | | member 2 (PLEKHH2), | |
| GNG11 | GNG11, GNGT11 | guanine nucleotide binding protein 11 | NM_004126 |
| CDH16 | CDH16 | cadherin 16, KSP-cadherin | NM_004062 |
| AKR1C1 | AKR1C1, C9, DD1, DDH, DDH1, H- | aldo-keto reductase family 1, member C1 | NM_001353 |
| | 37, MBAB, HAKRC, MGC8954, 2- | (dihydrodiol dehydrogenase 1; 20-alpha (3- | |
| | ALPHA-HSD, 20-ALPHA-HSD | alpha)-hydroxysteroid dehydrogenase) | |
| MGC42367 | MGC42367, C2orf55 | similar to 2010300C02Rik protein | NM_207362 |
| AQP1 | AQP1, CO, CHIP28, AQP- | Homo sapiens aquaporin 1 (channel-forming | NM_198098 |
| | CHIP, MGC26324 | integral protein, 28 kDa) (AQP1), transcript | |
| | | variant 1, | |
| RAFTLIN | RAFTLIN, MIG2, PIG9, PIB10, KIAA0084, | raft-linking protein | NM_015150 |
| | MGC141678, RFTN1 | | |
| FAM111A | FAM111A, FLJ22794, KIAA1895, | hypothetical protein FLJ22794 | NM_022074 |
| | DKFZp686A06175 | | |
| ADAMTS1 | ADAMTS1, C3-C5, | a disintegrin-like and metalloprotease (reprolysin | NM_006988 |
| | METH1, KIAA1346 | type) with thrombospondin type 1 motif, 1 | |
| FHOD3 | FHOD3, FHOS2, Formactin2 | hypothetical protein FLJ22297 | NM_025135 |
| DUSP23 | DUSP23, VHZ, LDP-3, | hypothetical protein FLJ20442 | NM_017823 |
| | DUSP25, FLJ20442, RP11-190A12, 1 | | |
| ITGB8 | ITGB8 | Homo sapiens, clone IMAGE: 4794726, | BC042028 |
| | | | NM_002214 |
| THC2134488 | THC2134488, THC2608799 | Unknown | |
| IL15 | IL15, IL-15, MGC9721 | Homo sapiens interleukin 15 (IL15), transcript | NM_172174 |
| | | variant 1, | |
| IGFBP3 | IGFBP3, IBP3, BP-53 | insulin-like growth factor binding protein 3 | NM_000598 |
| | | | NM_001013398 |
| CYP1A1 | CYP1A1, AHH, AHRR, CP11, CYP1, | cytochrome P450, subfamily I (aromatic | NM_000499 |
| | P1-450, P450-C, P450DX | compound-inducible), polypeptide 1 | |
| PHLDA1 | PHLDA1, PHRIP, TDAG51, DT1P1B11, | pleckstrin homology-like domain, family A, | AF220656 |
| | MGC131738 | member 1 | NM_007350 |
| TLR3 | TLR3, CD283 | toll-like receptor 3 | NM_003265 |
| GPC3 | GPC3, SGB, DGSX, SDYS, SGBS, SGBS1 | glypican 3 | NM_004484 |
| AHRR | AHRR, AHH, AHHR, KIAA1234 | Homo sapiens aryl-hydrocarbon receptor | NM_020731 |
| | | represser (AHRR), | |
| CACNG6 | CACNG6 | Homo sapiens calcium channel, voltage- | NM_145814 |
| | | dependent, gamma subunit 6 (CACNG6), | |
| | | transcript variant 1, | |
| AKR1C3 | AKR1C3, DD3, HAKRB, HAKRe, HA1753, | aldo-keto reductase family 1, member C3 (3- | NM_003739 |
| | HSD17B5, hluPGFS, KIAA0119 | alpha hydroxysteroid dehydrogenase, type II) | |
| FSTL1 | FSTL1, FRP, FSL1, Follistatin-like | follistatin-like 1 | NM_007085 |
| AHR | AHR | aryl hydrocarbon receptor | NM_001621 |
| C1orf88 | C1orf88, FLJ23853, MGC126550, | Homo sapiens hypothetical protein LOC128344 | NM_181643 |
| | RP5-1125M8, 4 | (LOC128344), | |
| CDKN1C | CDKN1C, BWS, WBS, p57, BWCR, KIP2 | cyclin-dependent kinase inhibitor 1C (p57, Kip2) | NM_000076 |
| A_32_P32463 | A_32_P32463, THC2753543 | Unknown | |
| PCDH9 | PCDH9 | protocadherin 9 | NM_020403 |
| NTN4 | NTN4, PRO3091, FLJ23180 | netrin 4 | NM_021229 |
| MID1 | MID1, OS, FXY, OSX, OGS1, XPRF, BBBG1, | midline 1 (Opitz/BBB syndrome) | NM_033290 |
| | GBBB1, RNF59, ZNFXY, TRIM18 | | |
| CSPG2 | CSPG2, VERSICAN, DKFZp686K06110, | chondroitin sulfate proteoglycan 2 (versican) | NM_004385 |
| | VCAN | | |
| AL133090 | AL133090, ADAM22 | Homo sapiens mRNA; cDNA DKFZp434E0528 | AL133090 |
| | | (from clone DKFZp434E0528), [AL133090] | |
| | | ADAM 22 precursor (A disintegrin and | |
| | | metalloproteinase domain 22) | |
| | | (Metalloproteinase-like, disintegrin-like, and | |
| | | cysteine-rich protein 2) (Metalloproteinase- | |
| | | disintegrin ADAM22-3). | |
| | | [Source: Uniprot/SWISSPROT; Acc: Q9P0K1] | |
| | | [ENST00000398204] | |
| DDC | DDC, AADC | dopa decarboxylase (aromatic L-amino acid | NM_000790 |
| | | decarboxylase) | |
| KCNH2 | KCNH2, ERG1, HERG, LQT2, SQT1, | potassium voltage-gated channel, subfamily H | NM_000238 |
| | HERG1, Kv11, 1 | (eag-related), member 2 | |
| ST6GAL1 | ST6GAL1, CD75, SIAT1, ST6Gall, | Homo sapiens sialyltransferase 1 (beta- | NM_173216 |
| | MGC48859, ST6Gal I | galactoside alpha-2,6-sialyltransferase) (SIAT1), | |
| | | transcript variant 1, | |
| TMEFF1 | TMEFF1, H7365, C9orf2 | transmembrane protein with EGF-like and two | NM_003692 |
| | | follistatin-like domains 1 | |
| FBXL16 | FBXL16, Fbl16, C16orf22, FLJ33735, | Homo sapiens F-box and leucine-rich repeat | NM_153350 |
| | MGC33974, c380A1, 1 | protein 16 (FBXL16), | |
| ATP8A2 | ATP8A2, IB, ATP, ML- | ATPase, aminophospholipid transporter-like, | AL390129 |
| | 1, ATPIB, DKFZP434B1913 | Class I, type 8A, member 2 | |
| CART1 | CART1, ALX1 | cartilage paired-class homeoprotein 1 | NM_006982 |
| C9orf150 | C9orf150, bA3L8, 2, FLJ38505, FLJ90271, | Homo sapiens chromosome 9 open reading | NM_203403 |
| | HYST0841, MGC46502 | frame 150 (C9orf150), | |
| HDAC9 | HDAC9, HD7, HDAC, HDRP, MITR, HDAC7, | histone deacetylase 7B | NM_014707 |
| | HDAC7B, HDAC9B, HDAC9FL, | | |
| | KIAA0744, DKFZp779K1053 | | |
| GLS | GLS, GLS1, FLJ10358, KIAA0838, | glutaminase | NM_014905 |
| | DKFZp686O15119 | | |
| GATS | GATS, DKFZp686B07267 | opposite strand transcription unit to STAG3 | BC065200 |
| CNTNAP3 | CNTNAP3, CASPR3, CNTNAP3A, | cell recognition molecule CASPR3 | NM_033655 |
| | RP11-290L7, 1, RP11-138L21, 1 | | |
| PDE4B | PDE4B, DPDE4, PDEIVB, MGC126529, | phosphodiesterase 4B, cAMP-specific (dunce | L12686 |
| | DKFZp686F2182 | (Drosophila)-homolog phosphodiesterase E4) | NM_001037341 |
| DKFZp586I1420 | DKFZp586I1420 | Homo sapiens hypothetical protein | NR_002186 |
| | | DKFZp586I1420 (DKFZp586I1420) on | |
| | | chromosome 7 | |
| ZNRF2 | ZNRF2, RNF202 | Homo sapiens zinc and ring finger 2 (ZNRF2), | NM_147128 |
| FGF2 | FGF2, BFGF, FGFB, HBGH-2 | fibroblast growth factor 2 (basic) | NM_002006 |
| SP5 | SP5 | Homo sapiens Sp5 transcription factor (SP5), | NM_001003845 |
| LAMA2 | LAMA2, LAMM | laminin, alpha 2 (merosin, congenital muscular | NM_000426 |
| | | dystrophy) | |
| THC2056328 | THC2056328 | Unknown | |
| KIAA1666 | KIAA1666, DKFZp434H0735, | KIAA1666 protein | AL117509 |
| | RIMBP3 | | NM_015672 |
| A_23_P103951 | A_23_P103951 | Unknown | |
| IL1R1 | IL1R1, P80, IL1R, IL1RA, CD121A, | interleukin 1 receptor, type I | NM_000877 |
| | D2S1473, IL-1R-alpha | | |
| CD55 | CD55, CR, TC, DAF | decay accelerating factor for complement (CD55, | NM_000574 |
| | | Cromer blood group system) | |
| MANEAL | MANEAL, FLJ31434, MGC78681, | Homo sapiens hypothetical protein FLJ31434 | NM_152496 |
| | RP11-109P14, 3 | | NM_001031740 |
| AK026140 | AK026140 | Homo sapiens cDNA: FLJ22487 fis, clone | AK026140 |
| | | HRC10931, | |
| FXYD2 | FXYD2, HOMG2, ATP1G1, MGC12372 | FXYD domain-containing ion transport regulator 2 | NM_021603 |
| CD40 | CD40, p50, Bp50, CDW40, MGC9013, | tumor necrosis factor receptor superfamily, | NM_001250 |
| | TNFRSF5 | member 5 | |
| KIAA1505 | KIAA1505, CCDC146 | KIAA1505 protein | NM_020879 |
| | | Homo sapiens coiled-coil domain containing 146 | |
| | | (CCDC146) | |
| DEPDC6 | DEPDC6, DEP, 6, FLJ12428, FLJ13854, | hypothetical protein FLJ12428 | NM_022783 |
| | DKFZp564B1778 | | |
| PPP2R2C | PPP2R2C, PR52, IMYPNO, IMYPNO1, | Homo sapiens protein phosphatase 2 (formerly | NM_020416 |
| | MGC33570 | 2A), regulatory subunit B (PR 52), gamma | |
| | | isoform (PPP2R2C), transcript variant 1, | |
| NRP1 | NRP1, NRP, CD304, VEGF165R, | neuropilin 1 | NM_003873 |
| | DKFZp781F1414, DKFZp686A03134 | | |
| ARHGDIB | ARHGDIB, D4, GDIA2, GDID4, LYGDI, | Rho GDP dissociation inhibitor (GDI) beta | NM_001175 |
| | Ly-GDI, RAP1GN1 | | |
| RAI2 | RAI2 | retinoic acid induced 2 | NM_021785 |
| LOC284262 | LOC284262 | hypothetical protein LOC284262 | AL832945 |
| TXNRD3 | TXNRD3, TGR, TR2, TRXR3 | thioredoxin reductase 2 | AF171055 |
| | | | BC050032 |
| HIVEP1 | HIVEP1, CIRIP, MBP-1, | human immunodeficiency virus type I enhancer- | NM_002114 |
| | ZNF40, CRYBP1, PRDII-BF1 | binding protein 1 | |
| BC042017 | BC042017 | Homo sapiens, clone IMAGE: 5311842, | BC042017 |
| ABCB2 | TAP1, APT1, PSF1, ABC17, ABCB2, | transporter 1, ATP-binding cassette, sub-family B | NM_000593 |
| | RING4, TAP1N, D6S114E, FLJ26666, | (MDR/TAP) | |
| | TAP1*0102N | | |
| RASSF8 | RASSF8, HoJ-1, C12orf2 | Homo sapiens C12ORF2 mRNA for C12ORF2, | AB093206 |
| | | complete cds, | AY665468 |
| CR622072 | CR622072 | full-length cDNA clone CS0DF032YA11 of Fetal | CR622072 |
| | | brain of Homo sapiens (human), | |
| LITAF | LITAF, PIG7, CMT1C, SIMPLE, TP53I7, | LPS-induced TNF-alpha factor | NM_004862 |
| | FLJ38636, MGC116698, MGC116700, | | |
| | MGC116701, MGC125274, | | |
| | MGC125275, MGC125276 | | |
| IGF2 | IGF2, INSIGF, pp9974, C11orf43, | Homo sapiens putative insulin-like growth factor | NM_001007139 |
| | FLJ22066, FLJ44734 | II associated protein (LOC492304), | NM_000612 |
| CYR61 | CYR61, CCN1, GIG1, IGFBP10 | cysteine-rich, angiogenic inducer, 61 | NM_001554 |
| PHF15 | PHF15, JADE2, KIAA0239 | KIAA0239 protein | NM_015288 |
| ProSAPiP1 | ProSAPiP1, KIAA0552 | ProSAPiP1 protein | NM_014731 |
| THC2227602 | THC2227602 | O02979 (O02979) ORF2280 gene homolog | |
| | | (Fragment), partial (18%) [THC2227602] | |
| LOC389722 | LOC389722, RP11-29017, 1, | similar to cell recognition molecule CASPR3 | AK054645 |
| | RP11-138L21, 1 | | |
| ANKRD18A | ANKRD18A, KIAA2015 | Homo sapiens mRNA for KIAA2015 protein, | AB095935 |
| FBN1 | FBN1, FBN, SGS, WMS, MASS, | fibrillin 1 (Marfan syndrome) | NM_000138 |
| | MFS1, OCTD | | |
| RPS6KA2 | RPS6KA2, RSK, HU-2, RSK3, p90-RSK3, | ribosomal protein S6 kinase, 90 kD, polypeptide 2 | NM_021135 |
| | pp90RSK3, MAPKAPK1C, | | |
| | S6K-alpha, S6K-alpha2 | | |
| GRB10 | GRB10, RSS, IRBP, MEG1, | Homo sapiens growth factor receptor-bound | NM_001001555 |
| | GRB-IR, KIAA0207 | protein 10 (GRB10), transcript variant 4, | |
| AY336981 | AY336981, GTF2IRD2 | Homo sapiens transcription factor GTF2IRD2 | AY336981 |
| | | isoform 3 (GTF2IRD2) mRNA, complete cds, | BC061590 |
| | | alternatively spliced, | |
| SLPI | SLPI, ALP, MPI, ALK1, BLPI, HUSI, | secretory leukocyte protease inhibitor | NM_003064 |
| | WAP4, WFDC4, HUSI-I | (antileukoproteinase) | |
| THC2095463 | THC2095463 | Q8LQA6 (Q8LQA6) OJ1125_C04, 6 protein, | |
| | | partial (21%) [THC2095463] | |
| COL16A1 | COL16A1, 447AA, FP1572 | collagen, type XVI, alpha 1 | NM_001856 |
| GRAMD3 | GRAMD3, NS3TP2, FLJ21313 | hypothetical protein FLJ21313 | NM_023927 |
| PDGFB | PDGFB, SIS, SSV, PDGF2, | platelet-derived growth factor beta polypeptide | NM_002608 |
| | c-sis, FLJ12858 | (simian sarcoma viral (v-sis) oncogene homolog) | |
| FAM107B | FAM107B, C10orf45, FLJ45505, | hypothetical protein MGC11034 | NM_031453 |
| | MGC11034, MGC90261 | | |
| LOC440934 | LOC440934 | hypothetical gene supported by BC008048 | BC008048 |
| | | | CR593560 |
| VCX | VCX, VCX1, VCXB1, VCX-B1, | variable charge, X chromosome | NM_013452 |
| | VCX10R, VCX-10r, MGC118975 | | |
| LAMB3 | LAMB3, LAMNB1 | laminin, beta 3 (nicein (125 kD), kalinin (140 kD), | NM_000228 |
| | | BM600 (125 kD)) | NM_001017402 |
| CYR61 | CYR61, CCN1, GIG1, IGFBP10 | cysteine-rich, angiogenic inducer, 61 | NM_001554 |
| NCALD | NCALD, MGC33870, MGC74858 | neurocalcin delta | NM_032041 |
| WNT5A | WNT5A, hWNT5A | wingless-type MMTV integration site family, | NM_003392 |
| | | member 5A | |
| ABCC3 | ABCC3, MLP2, MRP3, ABC31, MOAT-D, | ATP-binding cassette, sub-family C | NM_003786 |
| | cMOAT2, EST90757 | (CFTR/MRP), member 3 | |
| GLIS1 | GLIS1, FLJ36155 | Homo sapiens GLIS family zinc finger 1 (GLIS1), | NM_147193 |
| JAG1 | JAG1, AGS, AHD, AWS, HJ1, CD339, | jagged 1 (Alagille syndrome) | NM_000214 |
| | JAGL1, MGC104644 | | |
| NRL | NRL, RP27, D14S46E | neural retina leucine zipper | NM_006177 |
| AGT | AGT, ANHU, SERPINA8 | angiotensinogen (serine (or cysteine) proteinase | NM_000029 |
| | | inhibitor, clade A (alpha-1 antiproteinase, | |
| | | antitrypsin), member 8) | |
| TMSB4X | TMSB4X, FX, TB4X, PTMB4, TMSB4 | Homo sapiens cDNA FLJ31414 fis, clone | AK055976 |
| | | NT2NE2000260, weakly similar to THYMOSIN | |
| | | BETA-4, | |
| CCPG1 | CCPG1, CPR8, KIAA1254 | Homo sapiens cell cycle progression 1 (CCPG1), | NM_020739 |
| ADRA2C | ADRA2C, ADRA2L2, ADRARL2, | adrenergic, alpha-2C-, receptor | NM_000683 |
| | ADRA2RL2, ALPHA2CAR | | |
| BM665539 | BM665539 | UI-E-CL1-afb-b-14-0-UI, s1 UI-E-CL1 Homo sapiens | BM665539 |
| | | cDNA clone UI-E-CL1-afb-b-14-0-UI 3′, | |
| TEX15 | TEX15, DKFZP434M2415 | testis expressed sequence 15 | NM_031271 |
| SEMA3B | SEMA3B, SemA, SEMA5, SEMAA, | sema domain, immunoglobulin domain (Ig), short | NM_004636 |
| | semaV, LUCA-1, FLJ34863 | basic domain, secreted, (semaphorin) 3B | |
| NYD-SP18 | NYD-SP18 | testes development-related NYD-SP18 | NM_032599 |
| ASNS | ASNS, TS11 | asparagine synthetase | NM_001673 |
| NFKBIZ | NFKBIZ, IKBZ, INAP, MAIL, FLJ30225, | molecule possessing ankyrin repeats induced by | NM_031419 |
| | FLJ34463 | lipopolysaccharide (MAIL), homolog of mouse | |
| AK096677 | AK096677 | Homo sapiens cDNA FLJ39358 fis, clone | AK096677 |
| | | PEBLM2004015, | |
| CA313037 | CA313037 | CA313037 UI-CF-FN0-aex-g-14-0-UI, s1 UI-CF- | CA313037 |
| | | FN0 Homo sapiens cDNA clone UI-CF-FN0-aex- | |
| | | g-14-0-UI 3′, | |
| PTPRM | PTPRM, RPTPM, RPTPU, PTPRL1, | protein tyrosine phosphatase, receptor type, M | NM_002845 |
| | hR-PTPu, R-PTP-MU | | |
| SLC1A1 | SLC1A1, EAAC1, EAAT3 | solute carrier family 1 (neuronal/epithelial high | NM_004170 |
| | | affinity glutamate transporter, system Xag), | |
| | | member 1 | |
| NQO1 | NQO1, DTD, QR1, DHQU, DIA4, | diaphorase (NADH/NADPH) (cytochrome b-5 | NM_000903 |
| | NMOR1, NMORI | reductase) | |
| A_24_P401051 | A_24_P401051 | Unknown | |
| GPR161 | GPR161, RE2, FLJ33952 | Homo sapiens cDNA FLJ33952 fis, clone | AK091271 |
| | | CTONG2018614, | |
| A_32_P32905 | A_32_P32905 | Unknown | |
| LOC389652 | LOC389652 | similar to asparagine synthetase; glutamine- | XM_372040 |
| | | dependent asparagine synthetase; TS11 cell | |
| | | cycle control protein | |
| SRGAP3 | SRGAP3, WRP, MEGAP, SRGAP2, | Homo sapiens SLIT-ROBO Rho GTPase | NM_014850 |
| | ARHGAP14, KIAA0411 | activating protein 3 (SRGAP3), | |
| PDE4D | PDE4D, DPDE3, STRK1, HSPDE4D, | phosphodiesterase 4D, cAMP-specific (dunce | NM_006203 |
| | PDE4DN2 | (Drosophila)-homolog phosphodiesterase E3) | |
| THC2055165 | THC2055165 | Unknown | |
| AK022020 | AK022020 | Homo sapiens cDNA FLJ11958 fis, clone | AK022020 |
| | | HEMBB1000996, | |
| MGAT4A | MGAT4A, GNT-IV, GNT-IVA | mannosyl (alpha-1,3-)-glycoprotein beta-1,4-N- | NM_012214 |
| | | acetylglucosaminyltransferase, isoenzyme A | |
| THC2201936 | THC2201936 | Q8WSI5 (Q8WSI5) Prophenol oxidase, partial | |
| | | (3%) [THC2201936] | |
| THC2091303 | THC2091303 | GRI1_HUMAN (Q9Y3R0) Glutamate receptor- | |
| | | interacting protein 1 (GRIP1 protein), partial | |
| | | (82%) [THC2091303] | |
| FCRL2 | FCRL2 | Homo sapiens hypothetical protein FLJ31052 | NM_152378 |
| | | (FLJ31052), | |
| PDE4DIP | PDE4DIP, MMGL, CMYA2, MGC75440, | Homo sapiens phosphodiesterase 4D interacting | NM_001002811 |
| | DKFZp781J054 | protein (myomegalin) (PDE4DIP), transcript | |
| | | variant 5, | |
| WBP5 | WBP5, DKFZp313K1940 | pp21 homolog | NM_016303 |
| PIM1 | PIM1, PIM | pim-1 oncogene | NM_002648 |
| NUPR1 | NUPR1, P8, COM1 | nuclear protein 1 | NM_012385 |
| SMAD7 | SMAD7, MADH7, MADH8, FLJ16482 | MAD (mothers against decapentaplegic, | NM_005904 |
| | | Drosophila) homolog 7 | |
| LOC63920 | LOC63920 | transposon-derived Buster3 transposase-like | NM_022090 |
| STAT1 | STAT1, ISGF-3, | signal transducer and activator of transcription 1, | NM_007315 |
| | STAT91, DKFZp686B04100 | 91 kD | |
| AK057151 | AK057151 | Homo sapiens cDNA FLJ32589 fis, clone | AK057151 |
| | | SPLEN2000443, | |
| PPM1H | PPM1H, ARHCL1, FLJ13253, | KIAA1157 protein | AB032983 |
| | KIAA1157 | | |
| BM806490 | BM806490 | AGENCOURT_6553853 NIH_MGC_71 | BM806490 |
| | | Homo sapiens cDNA clone IMAGE: 5555887 5′, | |
| AL390181 | AL390181, ENST00000400361 | Homo sapiens mRNA; cDNA DKFZp547J125 | AL390181 |
| | | (from clone DKFZp547J125), | |
| LOC150356 | LOC150356 | hypothetical protein BC012882 | BC012882 |
| WNT7B | WNT7B | Homo sapiens wingless-type MMTV integration | NM_058238 |
| | | site family, member 7B (WNT7B), | |
| BF210146 | BF210146 | 601874052F1 NIH_MGC_54 Homo sapiens | BF210146 |
| | | cDNA clone IMAGE: 4098852 5′, | |
| LRP11 | LRP11, MANSC3, FLJ14735, | hypothetical protein FLJ14735 | NM_032832 |
| | MGC39092, bA350J20, 3 | | |
| FGFR2 | FGFR2, BEK, JWS, CEK3, CFD1, | fibroblast growth factor receptor 2 (bacteria- | NM_022972 |
| | ECT1, KGFR, TK14, TK25, BFR-1, K-SAM | expressed kinase, keratinocyte growth factor | |
| | | receptor, craniofacial dysostosis 1, Crouzon | |
| | | syndrome, Pfeiffer syndrome, Jackson-Weiss | |
| | | syndrome) | |
| MT2A | MT2A, MT2 | metallothionein 2A | NM_005953 |
| BF378046 | BF378046 | BF378046 RC1-TN0151-270900-013-b06 | BF378046 |
| | | TN0151 Homo sapiens cDNA, | |
| BC037328 | BC037328 | Homo sapiens cDNA clone IMAGE: 5263455, | BC037328 |
| | | partial cds, | |
| ZNF323 | ZNF323, ZNF310P, FLJ23407, | hypothetical protein FLJ23407 | NM_030899 |
| | ZNF20-Lp, dJ874C20, 2 | | |
| TBC1D8 | TBC1D8, AD3, VRP, HBLP1 | vascular Rab-GAP/TBC-containing | NM_007063 |
| BLVRA | BLVRA, BLVR, BVRA | biliverdin reductase A | NM_000712 |

(bis): List of the additional over-expressed genes (at least two-
fold) in the docetaxel resistant cell-lines IGR-CaP1 at 200 nM.

| | | | |
|---|---|---|---|
| KRT7 | KRT7 | Homo sapiens keratin 7 | NM_005556 |
| RHOU | RHOU | Homo sapiens ras homolog gene family, member U | NM_021205 |
| SUSD4 | SUSD4 | Homo sapiens sushi domain containing 4 | NM_001037175 |
| | | (SUSD4), transcript variant 2 | |
| VCX3A | VCX3A | Homo sapiens variable charge, X-linked 3A | NM_016379 |
| ROBO3 | ROBO3 | Homo sapiens roundabout, axon guidance | NM_022370 |
| | | receptor, homolog 3 (Drosophila) | |
| TNFSF13 | TNFSF13 | Homo sapiens tumor necrosis factor (ligand) | NM_172088 |
| | | superfamily, member 13 (TNFSF13), transcript | |
| | | variant gamma | |
| AA532655 | AA532655 | nj17d09.s1 NCI_CGAP_Pr22 Homo sapiens | AA532655 |
| | | cDNA clone IMAGE: 9866093′ | |

TABLE 2

| | | | |
|---|---|---|---|
| Primary | | | |
| Sequence Name | Sequence Name(s) | Sequence Description | Accession # |

List of the under-expressed genes (at least two-fold)
in the docetaxel resistant IGR-CaP1 cell-lines.

| | | | |
|---|---|---|---|
| PIK3C3 | PIK3C3, Vps34, MGC61518 | phosphoinositide-3-kinase, class 3 | NM_002647 |
| SCARB1 | SCARB1, CLA1, SRB1, CLA-1, SR- | CD36 antigen (collagen type I receptor, | NM_005505 |
| | BI, CD36L1, MGC138242 | thrombospondin receptor)-like 1 | |
| ASGR1 | ASGR1, ASGPR, CLEC4H1, Hs, 12056 | asialoglycoprotein receptor 1 | NM_001671 |
| FLJ22659 | FLJ22659 | hypothetical protein FLJ22659 | AK026312 |
| ASMTL | ASMTL, ASTML, ASMTLX, ASMTLY | acetylserotonin O-methyltransferase-like | NM_004192 |
| ARHGAP10 | ARHGAP10, GRAF2, PS- | hypothetical protein FLJ20896 | NM_024605 |
| | GAP, FLJ20896, FLJ41791 | | |
| EDG6 | EDG6, LPC1, S1P4, SLP4, S1PR4 | endothelial differentiation, G-protein-coupled | NM_003775 |
| | | receptor 6 | |
| KCNC3 | KCNC3, KV3, 3, SCA13, KSHIIID | potassium voltage-gated channel, Shaw-related | NM_004977 |
| | | subfamily, member 3 | |
| MGC11332 | MGC11332 | hypothetical protein MGC11332 | NM_032718 |
| LOC339240 | LOC339240 | Homo sapiens keratin pseudogene (LOC339240) | NR_001443 |
| | | on chromosome 17 | |
| PTPN3 | PTPN3, PTPH1, DKFZp686N0569 | Homo sapiens mRNA; cDNA DKFZp686N0569 | CR749204 |
| | | (from clone DKFZp686N0569), | |
| AK123483 | AK123483 | Homo sapiens cDNA FLJ41489 fis, clone | AK123483 |
| | | BRTHA2004582, | |
| NRG1 | NRG1, GGF, HGL, HRG, NDF, ARIA, GGF2, | neuregulin 1 | NM_013962 |
| | HRG1, HRGA, SMDF | | |
| FAM80A | FAM80A, MGC47816, RP11- | Homo sapiens hypothetical protein MGC47816 | NM_173642 |
| | 157D18, 1 | (MGC47816), | |
| BAMBI | BAMBI, NMA | putative transmembrane protein | NM_012342 |
| SAMD8 | SAMD8, FLJ25082 | Homo sapiens sterile alpha motif domain | NM_144660 |
| | | containing 8 (SAMD8), | |
| KCNMB4 | KCNMB4 | potassium large conductance calcium-activated | NM_014505 |
| | | channel, subfamily M, beta member 4 | |
| SPG20 | SPG20, SPARTIN, TAHCCP1, | Homo sapiens spastic paraplegia 20, spartin | NM_015087 |
| | KIAA0610 | (Troyer syndrome) (SPG20), | |
| RGS16 | RGS16, RGS-R, A28-RGS14, A28- | regulator of G-protein signalling 16 | NM_002928 |
| | RGS14P | | |
| UGT2B7 | UGT2B7, UGT2B9 | UDP glycosyltransferase 2 family, polypeptide B7 | NM_001074 |
| TMPRSS4 | TMPRSS4, MT-SP2, TMPRSS3 | transmembrane protease, serine 4 | NM_019894 |
| RAB39B | RAB39B | Homo sapiens RAB39B, member RAS oncogene | NM_171998 |
| | | family (RAB39B), | |
| HSPH1 | HSPH1, HSP105A, HSP105B, | heat shock 105 kD | NM_006644 |
| | KIAA0201, NY-CO-25, DKFZp686M05240 | | |
| PSCDBP | PSCDBP, HE, B3- | pleckstrin homology, Sec7 and coiled/coil | NM_004288 |
| | 1, CASP, CYBR, CYTIP | domains, binding protein | |
| UGT2B11 | UGT2B11, MGC129611, MGC129612 | UDP glycosyltransferase 2 family, polypeptide B11 | NM_001073 |
| ZNF516 | ZNF516, HsT287 | KIAA0222 gene product | D86975 |
| CKB | CKB, B-CK, CKBB | creatine kinase, brain | NM_001823 |
| SLC7A8 | SLC7A8, LAT2, LPI-PC1 | Homo sapiens solute carrier family 7 (cationic | NM_182728 |
| | | amino acid transporter, y+ system), member 8 | |
| | | (SLC7A8), transcript variant 2, | |
| UGT2B28 | UGT2B28 | Homo sapiens UDP glycosyltransferase 2 family, | NM_053039 |
| | | polypeptide B28 (UGT2B28), mRNA, | |
| SEMA3C | SEMA3C, SemE, SEMAE | sema domain, immunoglobulin domain (Ig), short | NM_006379 |
| | | basic domain, secreted, (semaphorin) 3C | |
| PDK1 | PDK1 | pyruvate dehydrogenase kinase, isoenzyme 1 | NM_002610 |
| GATA2 | GATA2, NFE1B, MGC2306 | hypothetical protein MGC2306 | NM_032638 |
| THC2064535 | THC2064535 | Unknown | |
| PDLIM5 | PDLIM5, L9, ENH, LIM | LIM protein (similar to rat protein kinase C-binding | NM_006457 |
| | | enigma) | |
| BX538293 | BX538293 | Homo sapiens mRNA; cDNA DKFZp686F09157 | BX538293 |
| | | (from clone DKFZp686F09157), | |
| HYAL1 | HYAL1, NAT6, LUCA1, HYAL-1, | Homo sapiens hyaluronoglucosaminidase 1 | NM_153284 |
| | MGC45987 | (HYAL1), transcript variant 4, | |
| UGT2B4 | UGT2B4, UGT2B11 | UDP glycosyltransferase 2 family, polypeptide B4 | NM_021139 |
| THC2185385 | THC2185385 | Unknown | |
| FLJ10159 | FLJ10159, SOBP | hypothetical protein FLJ10159 | AK001021 |
| | | | NM_018013 |
| UGT2B10 | UGT2B10, MGC142209 | UDP glycosyltransferase 2 family, polypeptide B10 | NM_001075 |
| PRTFDC1 | PRTFDC1, HHGP, FLJ11888 | HHGP protein | NM_020200 |
| A_24_P575267 | A_24_P575267 | Unknown | |
| PTGES | PTGES, PGES, PIG12, PP102, PP1294, MGST- | prostaglandin E synthase | NM_004878 |
| | IV, MGST1L1, TP53I12, MGC10317, | | |
| | MGST1-L1 | | |
| DNAJC15 | DNAJC15, MCJ, HSD18, DNAJD1 | DNAJ domain-containing | NM_013238 |
| NPAS1 | NPAS1, MOP5, PASD5 | neuronal PAS domain protein 1 | NM_002517 |
| A_24_P478940 | A_24_P478940, THC2668815 | Unknown | |
| | | Q4TBH3_TETNG (Q4TBH3) Chromosome 13 | |
| | | SCAF7124, whole genome shotgun sequence, | |
| | | partial (3%) [THC2668815] | |
| FRMD4B | FRMD4B, GRSP1, KIAA1013, | Homo sapiens FERM domain containing 4B, | BC028291 |
| | 6030440G0SRik | mRNA (cDNA clone IMAGE: 4508579), partial cds, | |
| SLC7A11 | SLC7A11, xCT, CCBR1 | Homo sapiens solute carrier family 7, (cationic | BC041925 |
| | | amino acid transporter, y+ system) member 11, | |
| | | mRNA (cDNA clone IMAGE: 5300264), partial cds, | |
| AF132203 | AF132203 | Homo sapiens PRO1933 mRNA, complete cds, | AF132203 |
| FLJ43855 | FLJ43855 | similar to sodium- and chloride-dependent | NM_198857 |
| | | creatine transporter | |
| TFDP2 | TFDP2, DP2, Dp-2 | transcription factor Dp-2 (E2F dimerization | NM_006286 |
| | | partner 2) | CR613689 |
| PFKFB4 | PFKFB4 | 6-phosphofructo-2-kinase/fructose-2,6- | NM_004567 |
| | | biphosphatase 4 | |
| TAC3 | TAC3, NKB, NKNB, PRO1155, ZNEUROK1 | tachykinin 3 (neuromedin K, neurokinin beta) | NM_013251 |
| ENC1 | ENC1, NRPB, CCL28, ENC- | ectodermal-neural cortex (with BTB-like domain) | NM_003633 |
| | 1, PIG10, TP53I10, FLJ39259 | | |
| NRP2 | NRP2, NP2, NPN2, PRO2714, | Homo sapiens neuropilin 2 (NRP2), transcript | NM_201264 |
| | MGC126574, VEGF165R2 | variant 6, | |
| MFHAS1 | MFHAS1, MASL1, FLJ23354 | MFH-amplified sequences with leucine-rich | NM_004225 |
| | | tandem repeats 1 | |
| AK024680 | AK024680 | Homo sapiens cDNA: FLJ21027 fis, clone | AK024680 |
| | | CAE07110, | |
| AL137342 | AL137342 | Homo sapiens mRNA; cDNA DKFZp761G1111 | AL137342 |
| | | (from clone DKFZp761G1111), | |
| D4S234E | D4S234E, P21, NSG1, D4S234, NEEP21 | DNA segment on chromosome 4 (unique) 234 | NM_014392 |
| | | expressed sequence | |
| LCP1 | LCP1, CP64, PLS2, LC64P, FLJ25423, | lymphocyte cytosolic protein 1 (L-plastin) | NM_002298 |
| | FLJ26114, FLJ39956, | | |
| | L-PLASTIN, DKFZp781A23186 | | |
| A_32_P95067 | A_32_P95067, DA571147 | Unknown | DA571147 |
| THC2038567 | THC2038567, LOC165186 | Q7UFH6 (Q7UFH6) Acetyl-CoA carboxylase | NM_199280 |
| | | (Biotin carboxyl carrier subunit) accB, partial | |
| | | (16%) [THC2038567] | |
| LMO1 | LMO1, TTG1, RBTN1, RHOM1, | LIM domain only 1 (rhombotin 1) | NM_002315 |
| | MGC116692 | | |
| BSPRY | BSPRY, FLJ20150 | hypothetical protein FLJ20150 | NM_017688 |
| BDNF | BDNF, MGC34632 | Homo sapiens brain-derived neurotrophic factor | NM_170735 |
| | | (BDNF), transcript variant 1, | |
| LIPG | LIPG, EL, EDL, PRO719 | lipase, endothelial | NM_006033 |
| AW205591 | AW205591, THC2665111 | UI-H-BI1-afr-b-02-0-UI, s1 NCI_CGAP_Sub3 | AW205591 |
| | | Homo sapiens cDNA clone IMAGE: 2722515 3′, | |
| AKAP12 | AKAP12, AKAP250, DKFZp686M0430, | A kinase (PRKA) anchor protein (gravin) 12 | NM_005100 |
| | DKFZp686O0331 | | NM_144497 |
| B3GALT1 | B3GALT1, MGC126594, beta3Gal-T1 | UDP-Gal:betaGlcNAc beta 1,3- | NM_020981 |
| | | galactosyltransferase, polypeptide 1 | |
| CHST7 | CHST7, C6ST-2 | carbohydrate (N-acetylglucosamine 6-O) | NM_019886 |
| | | sulfotransferase 7 | |
| NMNAT2 | NMNAT2, PNAT2, PNAT- | Homo sapiens nicotinamide nucleotide | NM_015039 |
| | 2, C1orf15, MGC2756, KIAA0479 | adenylyltransferase 2 (NMNAT2), transcript | |
| | | variant 1, | |
| SLC12A3 | SLC12A3, TSC, NCCT | solute carrier family 12 (sodium/chloride | NM_000339 |
| | | transporters), member 3 | |
| SLC22A2 | SLC22A2, OCT2, MGC32628 | solute carrier family 22 (organic cation | NM_003058 |
| | | transporter), member 2 | |
| LMBRD2 | LMBRD2, MGC125692, DKFZp434H2226, | Homo sapiens hypothetical protein | NM_001007527 |
| | DKFZp686G1057 | DKFZp434H2226 (DKFZp434H2226), mRNA | |
| AMPH | AMPH, AMPH1 | amphiphysin (Stiff-Mann syndrome with breast | NM_001635 |
| | | cancer 128 kD autoantigen) | |
| ANKRD37 | ANKRD37, Lrp2bp, MGC111507 | Homo sapiens low density lipoprotein receptor- | NM_181726 |
| | | related protein binding protein (Lrp2bp), | |
| LIN7A | LIN7A, LIN7, VELI1, LIN-7A, MALS- | Vertebrate LIN7 homolog 1, Tax interaction | NM_004664 |
| | 1, TIP-33, MGC148143 | protein 33 | |
| PHEX | PHEX, HYP, PEX, XLH, HPDR, HYP1, | phosphate regulating gene with homologies to | NM_000444 |
| | HPDR1 | endopeptidases on the X chromosome | |
| | | (hypophosphatemia, vitamin D resistant rickets) | |
| C1QL1 | C1QL1, CRF, C1QRF | C1q-related factor | NM_006688 |
| EPAS1 | EPAS1, HLF, MOP2, HIF2A, PASD2 | endothelial PAS domain protein 1 | NM_001430 |
| KCNC4 | KCNC4, KV3, 4, KSHIIIC, HKSHIIIC, | Homo sapiens potassium voltage-gated channel, | NM_153763 |
| | MGC126818 | Shaw-related subfamily, member 4 (KCNC4), | |
| | | transcript variant 2, | |
| FOXL2 | FOXL2, BPES, PFRK, POF3, BPES1, | forkhead box L2 | NM_023067 |
| | PINTO | | |
| SCD | SCD, SCD1, FADS5, PRO0998 | stearoyl-CoA desaturase (delta-9-desaturase) | NM_005063 |
| FGFBP1 | FGFBP1, FGFBP, HBP17 | heparin-binding growth factor binding protein | NM_005130 |
| CASP8 | CASP8, CAP4, MACH, MCH5, FLICE, | caspase 8, apoptosis-related cysteine protease | NM_033356 |
| | MGC78473 | | |
| LZTS1 | LZTS1, F37, FEZ1 | leucine zipper, putative tumor suppressor 1 | NM_021020 |
| SYTL3 | SYTL3, SLP3, MGC105130, MGC118883, | Homo sapiens synaptotagmin-like 3 (SYTL3), | NM_001009991 |
| | MGC118884, MGC118885 | | |
| HSHPX5 | HSHPX5, MSX2P1 | PREDICTED: Homo sapiens HPX-5 (HSHPX5), | XM_496232 |
| | | | NR_002307 |
| NLGN1 | NLGN1, KIAA1070, MGC45115 | neuroligin 1 | NM_014932 |
| MGST1 | MGST1, MGST, GST12, MGST-I, | Homo sapiens microsomal glutathione S- | NM_145791 |
| | MGC14525 | transferase 1 (MGST1), transcript variant 1c, | |
| ST8SIA4 | ST8SIA4, PST, PST1, SIAT8D, | sialyltransferase 8 (alpha-2, 8- | NM_005668 |
| | MGC34450, MGC61459, ST8SIA-IV | polysialytransferase) D | |
| THC2050576 | THC2050576, THC2634493 | Unknown | |
| SLC3A1 | SLC3A1, D2H, ATR1, NBAT, RBAT, | solute carrier family 3 (cystine, dibasic and | NM_000341 |
| | CSNU1, FLJ34681 | neutral amino acid transporters, activator of | |
| | | cystine, dibasic and neutral amino acid transport), | |
| | | member 1 | |
| TNRC9 | TNRC9, CAGF9 | trinucleotide repeat containing 9 | U80736 |
| AK022997 | AK022997 | Homo sapiens cDNA FLJ12935 fis, clone | AK022997 |
| | | NT2RP2004982, | |
| UGT8 | UGT8, CGT | UDP glycosyltransferase 8 (UDP-galactose | U62899 |
| | | ceramide galactosyltransferase) | |
| LETM2 | LETM2, FLJ25409 | Homo sapiens leucine zipper-EF-hand containing | NM_144652 |
| | | transmembrane protein 2 (LETM2), | |
| DIAPH2 | DIAPH2, DIA, POF, DIA2, POF2, FLJ11167 | diaphanous (Drosophila, homolog) 2 | NM_007309 |
| BC014452 | BC014452 | Homo sapiens cDNA clone IMAGE: 4903661, | BC014452 |
| | | complete cds, | |
| SUNC1 | SUNC1, MGC33329 | Homo sapiens Sad1 and UNC84 domain | NM_152782 |
| | | containing 1 (SUNC1), | NM_001030019 |
| DUSP13 | DUSP13, BEDP, MDSP, TMDP, SKRP4, | protein phosphatase | NM_016364 |
| | FLJ32450 | | NM_001007271 |
| AUTS2 | AUTS2, KIAA0442, MGC13140 | Homo sapiens autism susceptibility candidate 2 | NM_015570 |
| | | (AUTS2), | |
| PLAC8 | PLAC8, C15, onzin | hypothetical protein | NM_016619 |
| THC2210862 | THC2210862 | Unknown | |
| MSX2 | MSX2, FPP, MSH, PFM, CRS2, HOX8, | msh (Drosophila) homeo box homolog 2 | NM_002449 |
| | PFM1 | | |
| SMAD9 | SMAD9, MADH6, MADH9, SMAD8A, | MAD (mothers against decapentaplegic, | NM_005905 |
| | SMAD8B | Drosophila) homolog 9 | BM8022662 |
| TTN | TTN, TMD, CMH9, CMD1G, CMPD4, | Homo sapiens titin (TTN), transcript variant N2-A, | NM_133378 |
| | HMERF, LGMD2J, FLJ26020, FLJ26409, | | |
| | FLJ32040, FLJ34413, FLJ39564, | | |
| | FLJ43066, DKFZp451N061 | | |
| LRRN6C | LRRN6C, LERN3, LINGO2, FLJ31810 | Homo sapiens hypothetical protein FLJ31810 | NM_152570 |
| | | (FLJ31810), | |
| MEIS2 | MEIS2, MRG1, MGC2820, HsT18361 | Homo sapiens Meis1, myeloid ecotropic viral | NM_170677 |
| | | integration site 1 homolog 2 (mouse) (MEIS2), | |
| | | transcript variant a, | |
| DHRS3 | DHRS3, SDR1, RDH17, Rsdr1, retSDR1 | short-chain dehydrogenase/reductase 1 | NM_004753 |
| OLR1 | OLR1, LOX1, CLEC8A, SCARE1 | oxidised low density lipoprotein (lectin-like) | NM_002543 |
| | | receptor 1 | |
| NSBP1 | NSBP1 | nucleosomal binding protein 1 | NM_030763 |
| MOXD1 | MOXD1, MOX, PRO5780, dJ248E1, 1, | Homo sapiens monooxygenase, DBH-like 1 | NM_015529 |
| | DKFZP564G202 | (MOXD1), | |
| DCAMKL1 | DCAMKL1, DCLK, KIAA0369, DCLK1 | doublecortin and CaM kinase-like 1 | NM_004734 |
| C12orf59 | C12orf59, FLJ31166, MGC111385 | hypothetical protein FLJ31166 | NM_153022 |
| A_23_P136857 | A_23_P136857 | Unknown | |
| BF675806 | BF675806 | 602083723F1 NIH_MGC_83 Homo sapiens | BF675806 |
| | | cDNA clone IMAGE: 4248004 5′, | |
| SLC44A5 | SLC44A5, CTL5, FLJ34081, MGC34032 | Homo sapiens hypothetical protein MGC34032 | NM_152697 |
| | | (MGC34032), | |
| SALL1 | SALL1, TBS, HSAL1, ZNF794 | sal (Drosophila)-like 1 | NM_002968 |
| GPR177 | GPR177, MRP, WLS, C1orf139, | hypothetical protein FLJ23091 | NM_024911 |
| | FLJ23091, MGC14878, MGC131760 | | |
| FZD8 | FZD8, FZ-8, hFZ8 | frizzled (Drosophila) homolog 8 | NM_031866 |
| THC2088463 | THC2088463 | ALU7_HUMAN (P39194) Alu subfamily SQ | |
| | | sequence contamination warning entry, partial | |
| | | (8%) [THC2088463] | |
| FLJ39502 | FLJ39502, FLJ26337, MGC134803, | hypothetical protein FLJ39502 | AK096821 |
| | CCDC141 | | |
| PROS1 | PROS1, PSA, PROS, PS21, PS22, | protein S (alpha) | NM_000313 |
| | PS23, PS24, PS25, PS 26, Protein | | |
| | S, protein Sa | | |
| PTPRD | PTPRD, HPTP, PTPD, HPTPD, | protein tyrosine phosphatase, receptor type, D | NM_002839 |
| | MGC119750, MGC119751, MGC119752, | | |
| | MGC119753, HPTP-DELTA, R-PTP- | | |
| | DELTA | | |
| MYB | MYB, efg, c-myb, c-myb_CDS | v-myb avian myeloblastosis viral oncogene | NM_005375 |
| | | homolog | |
| SLC16A10 | SLC16A10, TAT1, PRO0813 | hypothetical protein PRO0813 | NM_018593 |
| GJA7 | GJA7, CX45, DKFZp686P0738, GJC1 | gap junction protein, alpha 7, 45 kD (connexin 45) | NM_005497 |
| GAL | GAL, GALN, GLNN, MGC40167 | galanin-related peptide | NM_015973 |
| PLXNA2 | PLXNA2, OCT, PLXN2, FLJ11751, | Homo sapiens cDNA FLJ30634 fis, clone | AK055196 |
| | FLJ30634, KIAA0463 | CTONG2002453, | NM_025179 |
| PDE1A | PDE1A, HCAM1, HSPDE1A, MGC26303 | Homo sapiens phosphodiesterase 1A, | NM_001003683 |
| | | calmodulin-dependent (PDE1A), transcript variant 2, | |
| AW467174 | AW467174 | AW467174 ha35g06, x1 NCI_CGAP_Kid12 | AW467174 |
| | | Homo sapiens cDNA clone IMAGE: 2875738 3′ similar to | |
| | | gb:X60673_rna1 GTP:AMP | |
| | | PHOSPHOTRANSFERASE MITOCHONDRIAL | |
| | | (HUMAN);, | |
| PLAT | PLAT, TPA, T-PA, DKFZp686I03148 | plasminogen activator, tissue | NM_000930 |
| LOC441047 | LOC441047 | similar to Adenylate kinase isoenzyme 4, | XM_496720 |
| | | mitochondrial (ATP-AMP transphosphorylase) | |
| CXCR4 | CXCR4, FB22, HM89, LAP3, LCR1, | chemokine (C-X-C motif), receptor 4 (fusin) | NM_003467 |
| | NPYR, WHIM, CD184, LESTR, NPY3R, | | NM_001008540 |
| | NPYRL, HSY3RR, NPYY3R, D2S201E | | |
| AK3L1 | AK3L1, AK3, AK4 | adenylate kinase 3 | NM_013410 |
| | | | NM_001005353 |
| SMPDL3A | SMPDL3A, ASM3A, ASML3a, FLJ20177, | Homo sapiens sphingomyelin phosphodiesterase, | NM_006714 |
| | yR36GH4, 1 | acid-like 3A (SMPDL3A), mRNA [NM_006714] | |
| KIAA0960 | KIAA0960, THSD7A | KIAA0960 protein | AB023177 |
| | | | NM_015204 |
| LHFP | LHFP, MGC22429 | lipoma HMGIC fusion partner | NM_005780 |
| CPM | CPM | carboxypeptidase M | NM_001874 |
| A_24_P345290 | A_24_P345290, AL599817 | Unknown | AL599817 |
| PNOC | PNOC, PPNOC | prepronociceptin | NM_006228 |
| GALNT14 | GALNT14, GALNT15, FLJ12691, | hypothetical protein FLJ12691 | NM_024572 |
| | FLJ13977, GalNac-T10, GalNac-T14 | | |
| TM4SF1 | TM4SF1, L6, H-L6, M3S1, TAAL6 | Homo sapiens transmembrane 4 superfamily | NM_014220 |
| | | member 1 (TM4SF1), | |
| ZAR1 | ZAR1 | Homo sapiens zygote arrest 1 (ZAR1), mRNA | NM_175619 |
| | | [NM_175619] | |
| A_23_P10091 | A_23_P10091 | Unknown | |
| GLT8D2 | GLT8D2, FLJ31494 | gycosyltransferase | NM_031302 |
| RXFP1 | RXFP1, LGR7, LGR7, 1, LGR7, 2, LGR7, | Relaxin receptor 1 (Leucine-rich repeat- | BX647985 |
| | 10, MGC138347, MGC142177 | containing G protein-coupled receptor 7), | |
| | | [Source: Uniprot/SWISSPROT; Acc: Q9HBX9] | |
| | | [ENST00000343542] | |
| CGNL1 | CGNL1, JACOP, FLJ14957, KIAA1749, | hypothetical protein FLJ14957 | NM_032866 |
| | MGC138254 | | |
| AK094972 | AK094972, LOC440338 | Homo sapiens cDNA FLJ37653 fis, clone | AK094972 |
| | | BRHIP2010217, | |
| LRCH2 | LRCH2, KIAA1495, dA204F4, 4 | Homo sapiens leucine-rich repeats and calponin | NM_020871 |
| | | homology (CH) domain containing 2 (LRCH2), | |
| BM930757 | BM930757 | UI-E-EJ1-ajm-l-20-0-UI, r1 UI-E-EJ1 | BM930757 |
| | | Homo sapiens cDNA clone UI-E-EJ1-ajm-l-20-0-UI 5′, | |
| ATP8A1 | ATP8A1, ATPIA, ATPP2, ATPASEII, | Homo sapiens ATPase, aminophospholipid | NM_006095 |
| | MGC26327, MGC130042, MGC130043 | transporter (APLT), Class I, type 8A, member 1 | |
| | | (ATP8A1), | |
| SOX9 | SOX9, CMD1, SRA1, CMPD1 | SRY (sex determining region Y)-box 9 | NM_000346 |
| | | (campomelic dysplasia, autosomal sex-reversal) | |
| SLC39A8 | SLC39A8, BIGM103, LZT-Hs6 | up-regulated by BCG-CWS | NM_022154 |
| TMEM47 | TMEM47, BCMP1, TM4SF10, | brain cell membrane protein 1 | NM_031442 |
| | MGC32949, DKFZp564E153, | | |
| | DKFZP761J17121 | | |
| SLC10A4 | SLC10A4, P4, MGC29802 | Homo sapiens solute carrier family 10 | NM_152679 |
| | | (sodium/bile acid cotransporter family), member 4 | |
| | | (SLC10A4), | |
| EDG7 | EDG7, AK091731 | Homo sapiens cDNA FLJ34412 fis, clone | AK091731 |
| | | HEART2002432, | |
| ITGA2 | ITGA2, BR, GPla, CD49B, VLA- | integrin, alpha 2 (CD49B, alpha 2 subunit of VLA- | NM_002203 |
| | 2, VLAA2 | 2 receptor) | |
| SLC1A3 | SLC1A3, EA6, EAAT1, GLAST, | solute carrier family 1 (glial high affinity glutamate | NM_004172 |
| | GLAST1, FLJ25094 | transporter), member 3 | |
| PLCXD3 | PLCXD3 | Homo sapiens phosphatidylinositol-specific | NM_001005473 |
| | | phospholipase C, X domain containing 3 | |
| | | (PLCXD3), | |
| BF514799 | BF514799 | UI-H-BW1-anj-a-01-0-UI, s1 NCI_CGAP_Sub7 | BF514799 |
| | | Homo sapiens cDNA clone IMAGE: 3082272 3′, | |
| SLC16A12 | SLC16A12, MCT12 | Homo sapiens cDNA FLJ42911 fis, clone | AK124901 |
| | | BRHIP3024118, weakly similar to | NM_213606 |
| | | Monocarboxylate transporter 4, | |
| THC2208430 | THC2208430 | Unknown | |
| THC2182743 | THC2182743, LPAR3 | EDG7_HUMAN (Q9UBY5) Lysophosphatidic acid | NM_012152 |
| | | receptor Edg-7 (LPA receptor 3) (LPA-3), partial | |
| | | (36%) [THC2182743] | |
| C4orf18 | C4orf18, AD021, AD036, FLJ38155, | AD021 protein | NM_016613 |
| | DKFZp434L142 | | |
| ANKRD38 | ANKRD38, FLJ10884, KIAA0172, | Homo sapiens hypothetical protein LOC163782 | NM_181712 |
| | dJ1078M7, 1, RP5-1155K23, 5, KANK4 | (LOC163782), | |
| CALCRL | CALCRL, CRLR, CGRPR | calcitonin receptor-like | NM_005795 |
| hSHISA3 | LOC152573 | hypothetical protein BC012029 | BC012029 |
| | | | NM_001080505 |

bis: List of the additional under-expressed genes (at least two-
fold) in the docetaxel resistant cell-lines IGR-CaP1 at 200 nM

| | | | |
|---|---|---|---|
| PRKD3 | PRKD3 | Homo sapiens protein kinase D3 | NM_005813 |
| LRRC8C | LRRC8C | Homo sapiens leucine rich repeat containing 8 | NM_032270 |
| | | family, member C | |
| LIMA1 | LIMA1 | Homo sapiens LIM domain and actin binding 1 | NM_016357 |
| | | (LIMA1), transcript variant b | |
| LNX1 | LNX1 | Homo sapiens ligand of numb-protein X 1 | NM_032622 |
| BM129308 | BM129308 | if20d02.x1 Melton Normalized Human Islet 4 N4- | BM129308 |
| | | HIS 1 Homo sapiens cDNA clone | |
| | | IMAGE: 5677082 3 | |
| EBF3 | EBF3 | Homo sapiens early B-cell factor 3 | NM_001005463 |
| CD44 | CD44 | Homo sapiens CD44 molecule (Indian blood | NM_000610 |
| | | group) (CD44), transcript variant 1 | |
| THC2616558 | THC2616558 | MMU72521 neural variant mena+ protein | |
| | | {Mus musculus} (exp = −1; wgp = 0; cg = 0), partial (3%) | |

TABLE 3
List of the over-expressed genes by at least two fold in the docetaxel
resistant cell-lines.

| | | | | | | | | IGR-CaP1 at 5, 12, | | |
|---|---|---|---|---|---|---|---|---|---|---|
| | | | | | | | | 25, 50, 100 and 200 nM | | |
| | | | | | Log | | Fold | Log | | Fold |
| | Log(Ratio) for each dose | | | | (ratio) | Ratio | change | (Ratio) | Ratio | change |
| Primary Sequence Name | 50 nM | 25 nM | 12 nM | 5 nM | Mean | Mean | Mean | Mean | Mean | Mean |
| RPIB9 | 1.49 | 1.51 | 1.55 | 1.59 | 1.53 | 34.22 | 34.22 | 1.52 | 33.04 | 33.04 |
| CXCL2 | 1.45 | 1.28 | 1.96 | 1.28 | 1.49 | 30.98 | 30.98 | 1.12 | 13.21 | 13.21 |
| AL137761 | 1.18 | 1.13 | 1.53 | 1.54 | 1.35 | 22.17 | 22.17 | 1.26 | 18.34 | 18.34 |
| TFPI2 | 1.61 | 1.65 | 1.48 | 0.61 | 1.34 | 21.84 | 21.84 | 1.54 | 34.56 | 34.56 |
| THC2051204 | 1.32 | 1.21 | 1.34 | 1.34 | 1.30 | 20.11 | 20.11 | | | |
| TNF | 1.22 | 1.28 | 1.52 | 1.04 | 1.26 | 18.36 | 18.36 | 1.04 | 10.87 | 10.87 |
| ABCB1 | 1.21 | 1.19 | 1.20 | 1.20 | 1.20 | 15.83 | 15.83 | 1.20 | 15.80 | 15.80 |
| PURG | 1.34 | 1.23 | 1.11 | 1.07 | 1.19 | 15.39 | 15.39 | 1.24 | 17.46 | 17.46 |
| ADAMTS5 | 1.22 | 1.34 | 1.03 | 0.96 | 1.14 | 13.75 | 13.75 | | | |
| MCTP1 | 1.01 | 1.14 | 1.28 | 1.00 | 1.11 | 12.79 | 12.79 | 1.03 | 10.68 | 10.68 |
| SPTLC2L | 0.88 | 1.22 | 1.36 | 0.92 | 1.10 | 12.46 | 12.46 | | | |
| OAS3 | 1.15 | 1.13 | 1.09 | 0.97 | 1.08 | 12.16 | 12.16 | 0.95 | 9.00 | 9.00 |
| GAS1 | 1.22 | 1.30 | 0.98 | 0.59 | 1.02 | 10.47 | 10.47 | 0.80 | 6.31 | 6.31 |
| BIRC3 | 0.96 | 1.04 | 1.28 | 0.79 | 1.02 | 10.40 | 10.40 | 1.16 | 14.39 | 14.39 |
| BQ186674 | 0.76 | 0.95 | 1.30 | 1.03 | 1.01 | 10.28 | 10.28 | 0.90 | 7.96 | 7.96 |
| MAL | 0.88 | 1.00 | 1.10 | 0.98 | 0.99 | 9.79 | 9.79 | | | |
| UBXD3 | 1.23 | 1.12 | 0.88 | 0.74 | 0.99 | 9.78 | 9.78 | 0.88 | 7.54 | 7.54 |
| WNT2B | 0.95 | 0.96 | 0.97 | 1.01 | 0.97 | 9.42 | 9.42 | | | |
| BM716045 | 1.06 | 1.02 | 0.98 | 0.82 | 0.97 | 9.36 | 9.36 | | | |
| SFRP1 | 1.04 | 1.01 | 0.91 | 0.91 | 0.97 | 9.30 | 9.30 | 0.78 | 5.99 | 5.99 |
| PLEKHH2 | 1.12 | 0.92 | 0.86 | 0.84 | 0.93 | 8.61 | 8.61 | 0.81 | 6.43 | 6.43 |
| GNG11 | 1.10 | 1.15 | 0.81 | 0.53 | 0.90 | 7.88 | 7.88 | 0.89 | 7.71 | 7.71 |
| CDH16 | 1.27 | 1.15 | 0.71 | 0.44 | 0.89 | 7.84 | 7.84 | 0.94 | 8.71 | 8.71 |
| AKR1C1 | 1.00 | 1.10 | 0.82 | 0.66 | 0.89 | 7.84 | 7.84 | 0.92 | 8.34 | 8.34 |
| MGC42367 | 1.23 | 1.12 | 0.77 | 0.46 | 0.89 | 7.83 | 7.83 | 0.94 | 8.66 | 8.66 |
| AQP1 | 0.90 | 0.87 | 0.90 | 0.90 | 0.89 | 7.82 | 7.82 | 0.85 | 7.06 | 7.06 |
| RAFTLIN | 0.83 | 0.94 | 1.01 | 0.76 | 0.89 | 7.69 | 7.69 | 0.65 | 4.49 | 4.49 |
| FAM111A | 0.90 | 0.82 | 0.86 | 0.93 | 0.88 | 7.53 | 7.53 | 0.96 | 9.07 | 9.07 |
| ADAMTS1 | 1.06 | 0.93 | 0.83 | 0.65 | 0.87 | 7.39 | 7.39 | 0.81 | 6.50 | 6.50 |
| FHOD3 | 0.93 | 0.91 | 0.82 | 0.77 | 0.86 | 7.20 | 7.20 | 0.85 | 7.00 | 7.00 |
| DUSP23 | 0.93 | 0.89 | 0.75 | 0.81 | 0.85 | 7.00 | 7.00 | 0.80 | 6.29 | 6.29 |
| ITGB8 | 0.87 | 0.88 | 0.89 | 0.74 | 0.85 | 7.00 | 7.00 | 0.90 | 7.99 | 7.99 |
| THC2134488 | 0.97 | 0.89 | 0.78 | 0.73 | 0.84 | 6.92 | 6.92 | 0.70 | 5.06 | 5.06 |
| IL15 | 0.87 | 0.92 | 0.89 | 0.64 | 0.83 | 6.75 | 6.75 | 0.78 | 6.01 | 6.01 |
| IGFBP3 | 0.66 | 0.99 | 0.90 | 0.69 | 0.81 | 6.45 | 6.45 | 0.85 | 7.09 | 7.09 |
| CYP1A1 | 0.83 | 0.78 | 0.72 | 0.82 | 0.79 | 6.14 | 6.14 | | | |
| PHLDA1 | 0.75 | 0.72 | 0.76 | 0.83 | 0.77 | 5.86 | 5.86 | 0.74 | 5.53 | 5.53 |
| TLR3 | 0.68 | 0.75 | 1.07 | 0.55 | 0.76 | 5.79 | 5.79 | | | |
| GPC3 | 0.86 | 0.81 | 0.64 | 0.72 | 0.76 | 5.74 | 5.74 | 0.74 | 5.54 | 5.54 |
| AHRR | 0.83 | 0.79 | 0.67 | 0.74 | 0.76 | 5.73 | 5.73 | | | |
| CACNG6 | 0.96 | 0.96 | 0.58 | 0.54 | 0.76 | 5.72 | 5.72 | 0.76 | 5.71 | 5.71 |
| AKR1C3 | 0.79 | 0.93 | 0.66 | 0.59 | 0.74 | 5.53 | 5.53 | 0.64 | 4.38 | 4.38 |
| FSTL1 | 0.87 | 0.74 | 0.81 | 0.53 | 0.74 | 5.48 | 5.48 | | | |
| AHR | 0.82 | 0.81 | 0.74 | 0.57 | 0.73 | 5.40 | 5.40 | 0.83 | 6.78 | 6.78 |
| C1orf88 | 1.05 | 0.91 | 0.49 | 0.48 | 0.73 | 5.39 | 5.39 | 0.73 | 5.41 | 5.41 |
| CDKN1C | 0.68 | 0.82 | 0.70 | 0.72 | 0.73 | 5.38 | 5.38 | 0.69 | 4.90 | 4.90 |
| A_32_P32463 | 0.79 | 0.76 | 0.65 | 0.72 | 0.73 | 5.37 | 5.37 | 0.65 | 4.47 | 4.47 |
| PCDH9 | 0.53 | 0.77 | 0.78 | 0.81 | 0.72 | 5.31 | 5.31 | | | |
| NTN4 | 0.88 | 0.75 | 0.60 | 0.67 | 0.72 | 5.29 | 5.29 | 0.59 | 3.87 | 3.87 |
| MID1 | 0.60 | 0.74 | 0.93 | 0.61 | 0.72 | 5.28 | 5.28 | 0.81 | 6.42 | 6.42 |
| CSPG2 | 0.80 | 0.80 | 0.77 | 0.50 | 0.72 | 5.22 | 5.22 | 0.68 | 4.75 | 4.75 |
| AL133090 | 0.96 | 0.89 | 0.66 | 0.35 | 0.71 | 5.15 | 5.15 | 0.72 | 5.23 | 5.23 |
| DDC | 0.76 | 1.01 | 0.50 | 0.55 | 0.71 | 5.08 | 5.08 | | | |
| KCNH2 | 0.38 | 0.83 | 0.90 | 0.65 | 0.69 | 4.86 | 4.86 | | | |
| ST6GAL1 | 0.78 | 0.91 | 0.69 | 0.32 | 0.67 | 4.72 | 4.72 | 0.56 | 3.59 | 3.59 |
| TMEFF1 | 0.75 | 0.79 | 0.64 | 0.51 | 0.67 | 4.70 | 4.70 | 0.71 | 5.11 | 5.11 |
| KRT7 | | | | | | | | 0.71 | 5.10 | 5.10 |
| FBXL16 | 0.96 | 0.88 | 0.46 | 0.38 | 0.67 | 4.69 | 4.69 | 0.70 | 4.96 | 4.96 |
| ATP8A2 | 0.79 | 0.76 | 0.76 | 0.34 | 0.66 | 4.60 | 4.60 | | | |
| CART1 | 0.69 | 0.76 | 0.78 | 0.40 | 0.66 | 4.57 | 4.57 | 0.58 | 3.83 | 3.83 |
| C9orf150 | 0.57 | 0.62 | 0.69 | 0.75 | 0.66 | 4.53 | 4.53 | 0.76 | 5.81 | 5.81 |
| HDAC9 | 0.77 | 0.55 | 0.71 | 0.55 | 0.64 | 4.41 | 4.41 | 0.52 | 3.34 | 3.34 |
| GLS | 0.65 | 0.62 | 0.74 | 0.56 | 0.64 | 4.39 | 4.39 | 0.65 | 4.46 | 4.46 |
| GATS | 0.79 | 0.73 | 0.55 | 0.46 | 0.63 | 4.30 | 4.30 | | | |
| CNTNAP3 | 0.72 | 0.70 | 0.71 | 0.40 | 0.63 | 4.30 | 4.30 | 0.58 | 3.79 | 3.79 |
| PDE4B | 0.84 | 0.61 | 0.62 | 0.43 | 0.63 | 4.23 | 4.23 | 0.57 | 3.69 | 3.69 |
| DKFZp586I1420 | 0.63 | 0.66 | 0.65 | 0.56 | 0.62 | 4.20 | 4.20 | 0.61 | 4.09 | 4.09 |
| ZNRF2 | 0.64 | 0.60 | 0.66 | 0.58 | 0.62 | 4.18 | 4.18 | 0.54 | 3.48 | 3.48 |
| FGF2 | 0.73 | 0.79 | 0.57 | 0.39 | 0.62 | 4.17 | 4.17 | | | |
| SP5 | 0.72 | 0.65 | 0.64 | 0.47 | 0.62 | 4.17 | 4.17 | 0.51 | 3.21 | 3.21 |
| LAMA2 | 0.46 | 0.63 | 0.77 | 0.59 | 0.61 | 4.08 | 4.08 | 0.52 | 3.28 | 3.28 |
| THC2056328 | 0.59 | 0.57 | 0.76 | 0.51 | 0.61 | 4.05 | 4.05 | | | |
| KIAA1666 | 0.75 | 0.68 | 0.53 | 0.46 | 0.61 | 4.03 | 4.03 | 0.64 | 4.41 | 4.41 |
| A_23_P103951 | 0.73 | 0.67 | 0.61 | 0.41 | 0.60 | 4.02 | 4.02 | | | |
| IL1R1 | 0.39 | 0.62 | 0.77 | 0.63 | 0.60 | 4.00 | 4.00 | | | |
| CD55 | 0.74 | 0.66 | 0.59 | 0.39 | 0.59 | 3.93 | 3.93 | 0.61 | 4.09 | 4.09 |
| MANEAL | 0.75 | 0.70 | 0.55 | 0.37 | 0.59 | 3.93 | 3.93 | 0.71 | 5.09 | 5.09 |
| AK026140 | 0.68 | 0.61 | 0.52 | 0.57 | 0.59 | 3.93 | 3.93 | 0.55 | 3.58 | 3.58 |
| FXYD2 | 0.43 | 0.58 | 0.70 | 0.64 | 0.59 | 3.88 | 3.88 | | | |
| CD40 | 0.60 | 0.65 | 0.56 | 0.54 | 0.59 | 3.87 | 3.87 | | | |
| KIAA1505 | 0.76 | 0.65 | 0.62 | 0.31 | 0.59 | 3.86 | 3.86 | 0.60 | 3.99 | 3.99 |
| DEPDC6 | 0.65 | 0.59 | 0.68 | 0.42 | 0.59 | 3.85 | 3.85 | 0.57 | 3.68 | 3.68 |
| PPP2R2C | 0.45 | 0.51 | 0.69 | 0.68 | 0.58 | 3.82 | 3.82 | 0.55 | 3.56 | 3.56 |
| ROBO3 | | | | | | | | 0.53 | 3.35 | 3.35 |
| NRP1 | 0.44 | 0.60 | 0.70 | 0.58 | 0.58 | 3.80 | 3.80 | | | |
| ARHGDIB | 0.46 | 0.56 | 0.72 | 0.56 | 0.57 | 3.75 | 3.75 | 0.45 | 2.84 | 2.84 |
| RAI2 | 0.84 | 0.64 | 0.34 | 0.47 | 0.57 | 3.74 | 3.74 | 0.60 | 3.96 | 3.96 |
| LOC284262 | 0.70 | 0.74 | 0.52 | 0.31 | 0.57 | 3.69 | 3.69 | | | |
| TXNRD3 | 0.51 | 0.58 | 0.63 | 0.54 | 0.57 | 3.68 | 3.68 | 0.53 | 3.36 | 3.36 |
| HIVEP1 | 0.54 | 0.54 | 0.63 | 0.56 | 0.57 | 3.68 | 3.68 | | | |
| BC042017 | 0.38 | 0.65 | 0.72 | 0.51 | 0.57 | 3.67 | 3.67 | 0.60 | 3.94 | 3.94 |
| ABCB2 | 0.56 | 0.58 | 0.64 | 0.48 | 0.56 | 3.66 | 3.66 | 0.52 | 3.34 | 3.34 |
| RASSF8 | 0.64 | 0.59 | 0.52 | 0.49 | 0.56 | 3.63 | 3.63 | 0.56 | 3.59 | 3.59 |
| CR622072 | 0.58 | 0.47 | 0.64 | 0.54 | 0.56 | 3.62 | 3.62 | 0.52 | 3.34 | 3.34 |
| LITAF | 0.68 | 0.59 | 0.50 | 0.45 | 0.55 | 3.56 | 3.56 | 0.49 | 3.08 | 3.08 |
| IGF2 | 0.67 | 0.67 | 0.49 | 0.38 | 0.55 | 3.56 | 3.56 | 0.62 | 4.21 | 4.21 |
| CYR61 | 0.61 | 0.49 | 0.51 | 0.59 | 0.55 | 3.55 | 3.55 | | | |
| PHF15 | 0.55 | 0.55 | 0.56 | 0.54 | 0.55 | 3.54 | 3.54 | | | |
| ProSAPiP1 | 0.63 | 0.51 | 0.51 | 0.54 | 0.55 | 3.51 | 3.51 | | | |
| THC2227602 | 0.76 | 0.63 | 0.43 | 0.35 | 0.54 | 3.49 | 3.49 | | | |
| LOC389722 | 0.61 | 0.59 | 0.53 | 0.43 | 0.54 | 3.46 | 3.46 | 0.43 | 2.72 | 2.72 |
| ANKRD18A | 0.66 | 0.53 | 0.58 | 0.35 | 0.53 | 3.38 | 3.38 | 0.56 | 3.65 | 3.65 |
| FBN1 | 0.71 | 0.54 | 0.55 | 0.31 | 0.53 | 3.37 | 3.37 | | | |
| RPS6KA2 | 0.39 | 0.47 | 0.62 | 0.62 | 0.52 | 3.34 | 3.34 | | | |
| GRB10 | 0.61 | 0.47 | 0.53 | 0.47 | 0.52 | 3.33 | 3.33 | 0.56 | 3.60 | 3.60 |
| AY336981 | 0.51 | 0.45 | 0.54 | 0.59 | 0.52 | 3.31 | 3.31 | 0.47 | 2.92 | 2.92 |
| SLPI | 0.39 | 0.67 | 0.63 | 0.38 | 0.52 | 3.31 | 3.31 | 0.70 | 5.00 | 5.00 |
| THC2095463 | 0.61 | 0.51 | 0.53 | 0.40 | 0.52 | 3.27 | 3.27 | | | |
| COL16A1 | 0.57 | 0.63 | 0.50 | 0.35 | 0.51 | 3.26 | 3.26 | 0.58 | 3.76 | 3.76 |
| GRAMD3 | 0.55 | 0.52 | 0.55 | 0.43 | 0.51 | 3.26 | 3.26 | 0.47 | 2.95 | 2.95 |
| PDGFB | 0.45 | 0.46 | 0.54 | 0.59 | 0.51 | 3.24 | 3.24 | | | |
| FAM107B | 0.47 | 0.45 | 0.53 | 0.59 | 0.51 | 3.23 | 3.23 | 0.47 | 2.92 | 2.92 |
| LOC440934 | 0.41 | 0.58 | 0.65 | 0.40 | 0.51 | 3.22 | 3.22 | 0.60 | 3.94 | 3.94 |
| VCX | 0.37 | 0.54 | 0.56 | 0.56 | 0.51 | 3.21 | 3.21 | 0.67 | 4.72 | 4.72 |
| LAMB3 | 0.49 | 0.56 | 0.58 | 0.38 | 0.50 | 3.18 | 3.18 | 0.63 | 4.29 | 4.29 |
| RHOU | | | | | | | | 0.63 | 4.25 | 4.25 |
| SUSD4 | | | | | | | | 0.62 | 4.16 | 4.16 |
| VCX3A | | | | | | | | 0.62 | 4.13 | 4.13 |
| CYR61 | 0.54 | 0.46 | 0.46 | 0.53 | 0.50 | 3.14 | 3.14 | | | |
| NCALD | 0.60 | 0.50 | 0.42 | 0.47 | 0.50 | 3.14 | 3.14 | | | |
| WNT5A | 0.58 | 0.41 | 0.47 | 0.52 | 0.49 | 3.12 | 3.12 | 0.44 | 2.78 | 2.78 |
| ABCC3 | 0.69 | 0.54 | 0.41 | 0.33 | 0.49 | 3.10 | 3.10 | | | |
| GLIS1 | 0.61 | 0.50 | 0.45 | 0.40 | 0.49 | 3.09 | 3.09 | | | |
| JAG1 | 0.60 | 0.50 | 0.45 | 0.39 | 0.49 | 3.06 | 3.06 | 0.48 | 3.04 | 3.04 |
| NRL | 0.48 | 0.51 | 0.52 | 0.41 | 0.48 | 3.03 | 3.03 | 0.49 | 3.06 | 3.06 |
| AGT | 0.43 | 0.38 | 0.42 | 0.68 | 0.48 | 3.01 | 3.01 | 0.43 | 2.71 | 2.71 |
| TMSB4X | 0.38 | 0.42 | 0.59 | 0.48 | 0.47 | 2.93 | 2.93 | 0.44 | 2.77 | 2.77 |
| CCPG1 | 0.53 | 0.49 | 0.44 | 0.38 | 0.46 | 2.90 | 2.90 | 0.60 | 4.00 | 4.00 |
| ADRA2C | 0.45 | 0.44 | 0.51 | 0.45 | 0.46 | 2.88 | 2.88 | 0.42 | 2.61 | 2.61 |
| BM665539 | 0.39 | 0.39 | 0.53 | 0.50 | 0.45 | 2.84 | 2.84 | 0.42 | 2.66 | 2.66 |
| TEX15 | 0.44 | 0.41 | 0.53 | 0.41 | 0.45 | 2.82 | 2.82 | 0.57 | 3.75 | 3.75 |
| SEMA3B | 0.42 | 0.44 | 0.48 | 0.45 | 0.45 | 2.81 | 2.81 | 0.41 | 2.59 | 2.59 |
| NYD-SP18 | 0.51 | 0.51 | 0.38 | 0.39 | 0.45 | 2.79 | 2.79 | | | |
| ASNS | 0.50 | 0.37 | 0.48 | 0.43 | 0.44 | 2.79 | 2.79 | | | |
| NFKBIZ | 0.43 | 0.31 | 0.54 | 0.49 | 0.44 | 2.77 | 2.77 | 0.40 | 2.53 | 2.53 |
| AK096677 | 0.53 | 0.38 | 0.49 | 0.37 | 0.44 | 2.76 | 2.76 | 0.47 | 2.95 | 2.95 |
| CA313037 | 0.52 | 0.50 | 0.41 | 0.33 | 0.44 | 2.76 | 2.76 | | | |
| PTPRM | 0.45 | 0.43 | 0.45 | 0.42 | 0.44 | 2.75 | 2.75 | 0.39 | 2.45 | 2.45 |
| SLC1A1 | 0.52 | 0.44 | 0.36 | 0.43 | 0.44 | 2.74 | 2.74 | | | |
| NQO1 | 0.46 | 0.41 | 0.45 | 0.42 | 0.44 | 2.73 | 2.73 | 0.47 | 2.96 | 2.96 |
| A_24_P401051 | 0.36 | 0.38 | 0.51 | 0.48 | 0.43 | 2.72 | 2.72 | | | |
| GPR161 | 0.50 | 0.47 | 0.39 | 0.37 | 0.43 | 2.71 | 2.71 | | | |
| A_32_P32905 | 0.58 | 0.41 | 0.43 | 0.30 | 0.43 | 2.70 | 2.70 | | | |
| LOC389652 | 0.47 | 0.35 | 0.46 | 0.42 | 0.43 | 2.68 | 2.68 | | | |
| SRGAP3 | 0.36 | 0.35 | 0.59 | 0.41 | 0.43 | 2.67 | 2.67 | | | |
| PDE4D | 0.44 | 0.42 | 0.34 | 0.49 | 0.42 | 2.64 | 2.64 | | | |
| THC2055165 | 0.49 | 0.40 | 0.43 | 0.33 | 0.41 | 2.58 | 2.58 | | | |
| AK022020 | 0.52 | 0.44 | 0.34 | 0.33 | 0.41 | 2.55 | 2.55 | 0.42 | 2.63 | 2.63 |
| MGAT4A | 0.41 | 0.38 | 0.37 | 0.44 | 0.40 | 2.53 | 2.53 | 0.40 | 2.49 | 2.49 |
| THC2201936 | 0.47 | 0.36 | 0.42 | 0.36 | 0.40 | 2.52 | 2.52 | | | |
| THC2091303 | 0.47 | 0.48 | 0.33 | 0.32 | 0.40 | 2.51 | 2.51 | | | |
| FCRL2 | 0.49 | 0.40 | 0.38 | 0.32 | 0.40 | 2.51 | 2.51 | | | |
| PDE4DIP | 0.41 | 0.37 | 0.44 | 0.38 | 0.40 | 2.51 | 2.51 | | | |
| WBP5 | 0.41 | 0.45 | 0.39 | 0.35 | 0.40 | 2.50 | 2.50 | | | |
| PIM1 | 0.53 | 0.43 | 0.32 | 0.31 | 0.40 | 2.50 | 2.50 | | | |
| NUPR1 | 0.45 | 0.34 | 0.43 | 0.37 | 0.40 | 2.49 | 2.49 | | | |
| SMAD7 | 0.51 | 0.39 | 0.33 | 0.34 | 0.39 | 2.48 | 2.48 | | | |
| LOC63920 | 0.47 | 0.37 | 0.36 | 0.37 | 0.39 | 2.47 | 2.47 | 0.54 | 3.50 | 3.50 |
| STAT1 | 0.45 | 0.49 | 0.30 | 0.33 | 0.39 | 2.47 | 2.47 | | | |
| AK057151 | 0.51 | 0.35 | 0.36 | 0.34 | 0.39 | 2.46 | 2.46 | | | |
| PPM1H | 0.46 | 0.39 | 0.35 | 0.36 | 0.39 | 2.45 | 2.45 | | | |
| BM806490 | 0.40 | 0.39 | 0.40 | 0.36 | 0.39 | 2.45 | 2.45 | | | |
| AL390181 | 0.44 | 0.40 | 0.37 | 0.31 | 0.38 | 2.41 | 2.41 | 0.43 | 2.67 | 2.67 |
| LOC150356 | 0.40 | 0.36 | 0.41 | 0.34 | 0.38 | 2.38 | 2.38 | | | |
| WNT7B | 0.37 | 0.36 | 0.40 | 0.37 | 0.38 | 2.37 | 2.37 | | | |
| BF210146 | 0.41 | 0.32 | 0.37 | 0.39 | 0.37 | 2.36 | 2.36 | | | |
| LRP11 | 0.42 | 0.41 | 0.32 | 0.33 | 0.37 | 2.33 | 2.33 | | | |
| FGFR2 | 0.37 | 0.38 | 0.33 | 0.33 | 0.36 | 2.27 | 2.27 | | | |
| MT2A | 0.44 | 0.34 | 0.32 | 0.32 | 0.35 | 2.26 | 2.26 | | | |
| BF378046 | 0.38 | 0.31 | 0.37 | 0.33 | 0.35 | 2.25 | 2.25 | | | |
| BC037328 | 0.38 | 0.32 | 0.37 | 0.31 | 0.35 | 2.21 | 2.21 | | | |
| ZNF323 | 0.33 | 0.35 | 0.34 | 0.35 | 0.34 | 2.20 | 2.20 | | | |
| TBC1D8 | 0.41 | 0.31 | 0.31 | 0.33 | 0.34 | 2.18 | 2.18 | | | |
| BLVRA | 0.34 | 0.34 | 0.31 | 0.35 | 0.33 | 2.16 | 2.16 | | | |
| AA532655 | | | | | | | | 0.42 | 2.66 | 2.66 |
| TNFSF13 | | | | | | | | 0.39 | 2.48 | 2.48 |

TABLE 4
List of the under-expressed genes by at least two fold in the docetaxel
resistant cell-lines.

| | | | | | | | | IGR-CaP1 at 5, 12, | | |
|---|---|---|---|---|---|---|---|---|---|---|
| | | | | | | | | 25, 50, 100 and 200 nM | | |
| | | | | | Log | | | Log | | Fold |
| | Log(Ratio) for each dose | | | | (ratio) | Ratio | Fold change | (ratio) | Ratio | change |
| Primary Sequence Name | 50 nM | 25 nM | 12 nM | 5 nM | Mean | Mean | Mean | Mean | Mean | Mean |
| PIK3C3 | −0.32 | −0.32 | −0.33 | −0.34 | −0.33 | 0.47 | −2.12 | | | |
| SCARB1 | −0.39 | −0.32 | −0.35 | −0.35 | −0.35 | 0.45 | −2.24 | | | |
| ASGR1 | −0.32 | −0.39 | −0.40 | −0.39 | −0.38 | 0.42 | −2.38 | | | |
| FLJ22659 | −0.42 | −0.31 | −0.37 | −0.40 | −0.38 | 0.42 | −2.38 | | | |
| ASMTL | −0.32 | −0.39 | −0.46 | −0.35 | −0.38 | 0.42 | −2.40 | | | |
| ARHGAP10 | −0.46 | −0.37 | −0.31 | −0.39 | −0.38 | 0.41 | −2.41 | | | |
| EDG6 | −0.44 | −0.43 | −0.36 | −0.34 | −0.39 | 0.41 | −2.45 | | | |
| KCNC3 | −0.46 | −0.43 | −0.32 | −0.35 | −0.39 | 0.41 | −2.45 | | | |
| MGC11332 | −0.48 | −0.39 | −0.36 | −0.34 | −0.39 | 0.41 | −2.47 | | | |
| LOC339240 | −0.41 | −0.45 | −0.30 | −0.42 | −0.39 | 0.40 | −2.48 | | | |
| PTPN3 | −0.43 | −0.36 | −0.36 | −0.43 | −0.39 | 0.40 | −2.48 | | | |
| AK123483 | −0.39 | −0.38 | −0.42 | −0.41 | −0.40 | 0.40 | −2.51 | −0.41 | 0.39 | −2.58 |
| PRKD3 | | | | | | | | −0.41 | 0.39 | −2.58 |
| NRG1 | −0.52 | −0.40 | −0.37 | −0.32 | −0.40 | 0.40 | −2.51 | | | |
| FAM80A | −0.33 | −0.31 | −0.46 | −0.52 | −0.41 | 0.39 | −2.55 | | | |
| BAMBI | −0.55 | −0.36 | −0.32 | −0.40 | −0.41 | 0.39 | −2.56 | | | |
| SAMD8 | −0.56 | −0.44 | −0.32 | −0.36 | −0.42 | 0.38 | −2.62 | | | |
| KCNMB4 | −0.58 | −0.39 | −0.37 | −0.36 | −0.43 | 0.38 | −2.67 | | | |
| SPG20 | −0.53 | −0.45 | −0.35 | −0.40 | −0.43 | 0.37 | −2.70 | | | |
| RGS16 | −0.58 | −0.43 | −0.34 | −0.38 | −0.43 | 0.37 | −2.71 | | | |
| UGT2B7 | −0.65 | −0.45 | −0.31 | −0.34 | −0.44 | 0.37 | −2.74 | | | |
| TMPRSS4 | −0.68 | −0.39 | −0.35 | −0.37 | −0.45 | 0.36 | −2.79 | | | |
| RAB39B | −0.34 | −0.54 | −0.42 | −0.50 | −0.45 | 0.36 | −2.81 | | | |
| HSPH1 | −0.68 | −0.50 | −0.31 | −0.35 | −0.46 | 0.35 | −2.89 | | | |
| PSCDBP | −0.44 | −0.46 | −0.50 | −0.46 | −0.46 | 0.34 | −2.91 | −0.50 | 0.32 | −3.14 |
| UGT2B11 | −0.69 | −0.45 | −0.34 | −0.38 | −0.46 | 0.34 | −2.91 | | | |
| ZNF516 | −0.55 | −0.46 | −0.39 | −0.49 | −0.47 | 0.34 | −2.95 | | | |
| CKB | −0.58 | −0.49 | −0.36 | −0.46 | −0.47 | 0.34 | −2.98 | −0.49 | 0.33 | −3.06 |
| SLC7A8 | −0.34 | −0.42 | −0.61 | −0.56 | −0.48 | 0.33 | −3.05 | −0.55 | 0.28 | −3.57 |
| UGT2B28 | −0.76 | −0.50 | −0.34 | −0.37 | −0.49 | 0.32 | −3.10 | | | |
| SEMA3C | −0.59 | −0.56 | −0.35 | −0.47 | −0.49 | 0.32 | −3.11 | | | |
| PDK1 | −0.52 | −0.50 | −0.50 | −0.45 | −0.49 | 0.32 | −3.11 | −0.49 | 0.32 | −3.08 |
| GATA2 | −0.55 | −0.50 | −0.50 | −0.44 | −0.50 | 0.32 | −3.13 | −0.44 | 0.37 | −2.74 |
| THC2064535 | −0.52 | −0.45 | −0.51 | −0.51 | −0.50 | 0.32 | −3.16 | −0.47 | 0.34 | −2.96 |
| PDLIM5 | −0.60 | −0.53 | −0.42 | −0.45 | −0.50 | 0.32 | −3.16 | −0.52 | 0.31 | −3.28 |
| BX538293 | −0.54 | −0.56 | −0.39 | −0.52 | −0.50 | 0.31 | −3.18 | | | |
| HYAL1 | −0.70 | −0.56 | −0.36 | −0.40 | −0.50 | 0.31 | −3.20 | | | |
| UGT2B4 | −0.74 | −0.55 | −0.34 | −0.39 | −0.51 | 0.31 | −3.20 | | | |
| THC2185385 | −0.54 | −0.48 | −0.39 | −0.62 | −0.51 | 0.31 | −3.21 | | | |
| FLJ10159 | −0.56 | −0.49 | −0.46 | −0.53 | −0.51 | 0.31 | −3.24 | −0.54 | 0.29 | −3.51 |
| UGT2B10 | −0.75 | −0.54 | −0.35 | −0.41 | −0.51 | 0.31 | −3.26 | | | |
| PRTFDC1 | −0.49 | −0.50 | −0.64 | −0.42 | −0.51 | 0.31 | −3.26 | | | |
| A_24_P575267 | −0.78 | −0.50 | −0.36 | −0.42 | −0.51 | 0.31 | −3.26 | | | |
| PTGES | −0.68 | −0.59 | −0.40 | −0.38 | −0.51 | 0.31 | −3.27 | −0.55 | 0.28 | −3.51 |
| DNAJC15 | −0.78 | −0.57 | −0.35 | −0.37 | −0.52 | 0.30 | −3.29 | −0.55 | 0.28 | −3.57 |
| NPAS1 | −0.67 | −0.57 | −0.46 | −0.37 | −0.52 | 0.30 | −3.29 | −0.51 | 0.31 | −3.20 |
| LRRC8C | | | | | | | | −0.51 | 0.31 | −3.27 |
| A_24_P478940 | −0.70 | −0.58 | −0.40 | −0.39 | −0.52 | 0.30 | −3.30 | −0.56 | 0.27 | −3.65 |
| FRMD4B | −0.49 | −0.50 | −0.58 | −0.52 | −0.52 | 0.30 | −3.34 | | | |
| SLC7A11 | −0.58 | −0.62 | −0.39 | −0.52 | −0.53 | 0.30 | −3.36 | | | |
| AF132203 | −0.61 | −0.47 | −0.42 | −0.61 | −0.53 | 0.30 | −3.36 | | | |
| FLJ43855 | −0.61 | −0.54 | −0.50 | −0.46 | −0.53 | 0.30 | −3.38 | | | |
| TFDP2 | −0.67 | −0.65 | −0.46 | −0.33 | −0.53 | 0.30 | −3.38 | −0.55 | 0.28 | −3.53 |
| PFKFB4 | −0.54 | −0.56 | −0.51 | −0.50 | −0.53 | 0.30 | −3.38 | −0.52 | 0.30 | −3.30 |
| TAC3 | −0.55 | −0.46 | −0.50 | −0.61 | −0.53 | 0.30 | −3.38 | | | |
| ENC1 | −0.74 | −0.50 | −0.49 | −0.40 | −0.53 | 0.29 | −3.40 | −0.57 | 0.27 | −3.69 |
| NRP2 | −0.72 | −0.54 | −0.49 | −0.42 | −0.54 | 0.29 | −3.48 | −0.49 | 0.33 | −3.07 |
| MFHAS1 | −0.56 | −0.56 | −0.53 | −0.53 | −0.54 | 0.29 | −3.50 | −0.56 | 0.27 | −3.66 |
| AK024680 | −0.68 | −0.50 | −0.47 | −0.52 | −0.54 | 0.29 | −3.51 | −0.62 | 0.24 | −4.19 |
| BDNF | −0.63 | −0.46 | −0.50 | −0.62 | −0.55 | 0.28 | −3.57 | −0.55 | 0.28 | −3.58 |
| LIMA1 | | | | | | | | −0.56 | 0.28 | −3.59 |
| AL137342 | −0.80 | −0.41 | −0.41 | −0.64 | −0.56 | 0.27 | −3.67 | −0.66 | 0.22 | −4.59 |
| D4S234E | −0.69 | −0.44 | −0.46 | −0.68 | −0.57 | 0.27 | −3.70 | −0.66 | 0.22 | −4.61 |
| LCP1 | −0.92 | −0.52 | −0.30 | −0.54 | −0.57 | 0.27 | −3.72 | −0.60 | 0.25 | −3.96 |
| A_32_P95067 | −0.72 | −0.63 | −0.54 | −0.39 | −0.57 | 0.27 | −3.73 | −0.59 | 0.26 | −3.91 |
| THC2038567 | −0.86 | −0.75 | −0.38 | −0.32 | −0.57 | 0.27 | −3.75 | −0.68 | 0.21 | −4.74 |
| LMO1 | −0.61 | −0.56 | −0.61 | −0.52 | −0.58 | 0.27 | −3.77 | | | |
| BSPRY | −0.72 | −0.46 | −0.52 | −0.61 | −0.58 | 0.26 | −3.78 | | | |
| BDNF | −0.61 | −0.49 | −0.56 | −0.67 | −0.58 | 0.26 | −3.81 | | | |
| LIPG | −0.85 | −0.67 | −0.33 | −0.51 | −0.59 | 0.26 | −3.89 | | | |
| AW205591 | −0.74 | −0.79 | −0.52 | −0.32 | −0.59 | 0.26 | −3.90 | −0.65 | 0.23 | −4.43 |
| AKAP12 | −0.94 | −0.68 | −0.32 | −0.43 | −0.59 | 0.25 | −3.92 | −0.71 | 0.20 | −5.08 |
| B3GALT1 | −0.54 | −0.53 | −0.63 | −0.67 | −0.59 | 0.25 | −3.93 | | | |
| CHST7 | −0.79 | −0.64 | −0.47 | −0.49 | −0.59 | 0.25 | −3.93 | | | |
| NMNAT2 | −0.86 | −0.74 | −0.44 | −0.36 | −0.60 | 0.25 | −3.96 | −0.62 | 0.24 | −4.21 |
| SLC12A3 | −0.86 | −0.67 | −0.31 | −0.57 | −0.60 | 0.25 | −3.98 | −0.73 | 0.19 | −5.38 |
| SLC22A2 | −0.94 | −0.67 | −0.40 | −0.43 | −0.61 | 0.25 | −4.05 | −0.60 | 0.25 | −3.95 |
| LMBRD2 | −0.67 | −0.61 | −0.55 | −0.62 | −0.61 | 0.24 | −4.09 | | | |
| AMPH | −0.89 | −0.57 | −0.49 | −0.53 | −0.62 | 0.24 | −4.17 | | | |
| ANKRD37 | −0.76 | −0.66 | −0.48 | −0.59 | −0.62 | 0.24 | −4.18 | −0.56 | 0.27 | −3.66 |
| LIN7A | −0.98 | −0.59 | −0.39 | −0.52 | −0.62 | 0.24 | −4.19 | −0.77 | 0.17 | −5.83 |
| PHEX | −0.88 | −0.76 | −0.42 | −0.44 | −0.62 | 0.24 | −4.22 | −0.66 | 0.22 | −4.60 |
| C1QL1 | −0.59 | −0.55 | −0.72 | −0.64 | −0.63 | 0.24 | −4.24 | −0.69 | 0.20 | −4.91 |
| EPAS1 | −0.63 | −0.57 | −0.63 | −0.72 | −0.64 | 0.23 | −4.34 | −0.73 | 0.19 | −5.33 |
| KCNC4 | −0.75 | −0.67 | −0.50 | −0.64 | −0.64 | 0.23 | −4.36 | −0.59 | 0.26 | −3.92 |
| FOXL2 | −0.81 | −0.68 | −0.47 | −0.61 | −0.64 | 0.23 | −4.38 | | | |
| SCD | −0.77 | −0.58 | −0.62 | −0.61 | −0.64 | 0.23 | −4.40 | | | |
| FGFBP1 | −0.99 | −0.64 | −0.47 | −0.50 | −0.65 | 0.22 | −4.48 | −0.71 | 0.19 | −5.18 |
| CASP8 | −0.40 | −0.77 | −0.65 | −0.80 | −0.65 | 0.22 | −4.51 | | | |
| LZTS1 | −0.81 | −0.71 | −0.65 | −0.46 | −0.66 | 0.22 | −4.53 | −0.78 | 0.17 | −6.01 |
| SYTL3 | −0.74 | −0.81 | −0.54 | −0.53 | −0.66 | 0.22 | −4.54 | −0.78 | 0.17 | −5.97 |
| HSHPX5 | −0.80 | −0.61 | −0.63 | −0.59 | −0.66 | 0.22 | −4.56 | −0.64 | 0.23 | −4.41 |
| NLGN1 | −0.41 | −0.33 | −0.86 | −1.05 | −0.66 | 0.22 | −4.62 | | | |
| MGST1 | −0.98 | −0.68 | −0.45 | −0.58 | −0.67 | 0.21 | −4.70 | −0.77 | 0.17 | −5.90 |
| ST8SIA4 | −0.75 | −0.53 | −0.59 | −0.85 | −0.68 | 0.21 | −4.77 | | | |
| THC2050576 | −1.00 | −0.72 | −0.50 | −0.51 | −0.68 | 0.21 | −4.78 | −0.75 | 0.18 | −5.59 |
| SLC3A1 | −0.87 | −0.74 | −0.62 | −0.51 | −0.68 | 0.21 | −4.82 | −0.76 | 0.17 | −5.75 |
| TNRC9 | −0.82 | −0.62 | −0.51 | −0.79 | −0.68 | 0.21 | −4.84 | | | |
| AK022997 | −1.23 | −0.47 | −0.36 | −0.70 | −0.69 | 0.20 | −4.88 | | | |
| UGT8 | −0.94 | −0.51 | −0.56 | −0.76 | −0.69 | 0.20 | −4.93 | −0.76 | 0.18 | −5.69 |
| LETM2 | −1.04 | −0.83 | −0.41 | −0.50 | −0.70 | 0.20 | −4.97 | | | |
| DIAPH2 | −1.18 | −0.71 | −0.38 | −0.54 | −0.70 | 0.20 | −5.05 | | | |
| BC014452 | −0.91 | −0.61 | −0.56 | −0.76 | −0.71 | 0.20 | −5.12 | | | |
| SUNC1 | −0.95 | −0.79 | −0.60 | −0.52 | −0.72 | 0.19 | −5.20 | −0.77 | 0.17 | −5.83 |
| DUSP13 | −0.84 | −0.74 | −0.61 | −0.71 | −0.73 | 0.19 | −5.32 | −0.70 | 0.20 | −4.98 |
| LNX1 | | | | | | | | −0.71 | 0.20 | −5.07 |
| AUTS2 | −0.59 | −0.52 | −0.84 | −1.02 | −0.74 | 0.18 | −5.51 | −0.66 | 0.22 | −4.56 |
| PLAC8 | −1.15 | −0.84 | −0.43 | −0.55 | −0.74 | 0.18 | −5.52 | −0.86 | 0.14 | −7.17 |
| THC2210862 | −1.43 | −0.77 | −0.43 | −0.36 | −0.75 | 0.18 | −5.61 | | | |
| MSX2 | −0.96 | −0.69 | −0.72 | −0.68 | −0.76 | 0.17 | −5.79 | −0.80 | 0.16 | −6.25 |
| SMAD9 | −0.79 | −0.80 | −0.77 | −0.70 | −0.76 | 0.17 | −5.81 | −1.23 | 0.06 | −16.99 |
| TTN | −0.86 | −0.74 | −0.55 | −0.91 | −0.77 | 0.17 | −5.82 | −0.71 | 0.20 | −5.12 |
| BM129308 | | | | | | | | −0.73 | 0.19 | −5.31 |
| LRRN6C | −1.00 | −0.73 | −0.53 | −0.82 | −0.77 | 0.17 | −5.88 | −0.81 | 0.16 | −6.43 |
| MEIS2 | −1.12 | −0.92 | −0.67 | −0.38 | −0.77 | 0.17 | −5.91 | −0.74 | 0.18 | −5.44 |
| DHRS3 | −1.03 | −0.75 | −0.65 | −0.67 | −0.77 | 0.17 | −5.95 | −0.75 | 0.18 | −5.57 |
| OLR1 | −1.34 | −0.95 | −0.31 | −0.58 | −0.80 | 0.16 | −6.25 | −0.89 | 0.13 | −7.74 |
| NSBP1 | −1.18 | −0.84 | −0.55 | −0.64 | −0.80 | 0.16 | −6.30 | | | |
| MOXD1 | −0.90 | −0.70 | −0.80 | −0.82 | −0.80 | 0.16 | −6.37 | −0.77 | 0.17 | −5.83 |
| DCAMKL1 | −1.11 | −0.84 | −0.65 | −0.63 | −0.81 | 0.16 | −6.40 | −0.88 | 0.13 | −7.59 |
| C12orf59 | −1.01 | −0.81 | −0.64 | −0.76 | −0.81 | 0.16 | −6.42 | −0.71 | 0.20 | −5.12 |
| A_23_P136857 | −1.10 | −0.99 | −0.71 | −0.52 | −0.83 | 0.15 | −6.75 | | | |
| BF675806 | −1.09 | −0.87 | −0.72 | −0.65 | −0.83 | 0.15 | −6.82 | | | |
| SLC44A5 | −1.28 | −0.83 | −0.73 | −0.54 | −0.84 | 0.14 | −6.96 | | | |
| EBF3 | | | | | | | | −0.82 | 0.15 | −6.54 |
| SALL1 | −1.26 | −1.04 | −0.74 | −0.36 | −0.85 | 0.14 | −7.03 | −0.82 | 0.15 | −6.59 |
| GPR177 | −1.27 | −0.89 | −0.62 | −0.63 | −0.85 | 0.14 | −7.13 | | | |
| FZD8 | −0.94 | −0.79 | −0.85 | −0.93 | −0.88 | 0.13 | −7.52 | −0.86 | 0.14 | −7.30 |
| THC2088463 | −0.98 | −0.95 | −0.73 | −0.85 | −0.88 | 0.13 | −7.55 | | | |
| FLJ39502 | −0.87 | −0.97 | −0.69 | −1.01 | −0.88 | 0.13 | −7.65 | −0.65 | 0.22 | −4.47 |
| PROS1 | −1.02 | −0.87 | −0.69 | −0.97 | −0.89 | 0.13 | −7.76 | −0.86 | 0.14 | −7.26 |
| PTPRD | −1.28 | −0.82 | −0.66 | −0.85 | −0.90 | 0.13 | −7.97 | | | |
| MYB | −0.89 | −0.81 | −0.86 | −1.05 | −0.90 | 0.12 | −8.01 | −0.85 | 0.14 | −7.07 |
| SLC16A10 | −1.19 | −1.22 | −0.74 | −0.49 | −0.91 | 0.12 | −8.08 | −1.43 | 0.04 | −26.86 |
| GJA7 | −1.18 | −0.87 | −0.80 | −0.80 | −0.91 | 0.12 | −8.12 | −1.01 | 0.10 | −10.32 |
| GAL | −1.22 | −0.96 | −0.71 | −0.81 | −0.92 | 0.12 | −8.40 | −0.93 | 0.12 | −8.60 |
| CD44 | | | | | | | | −0.94 | 0.12 | −8.65 |
| PLXNA2 | −1.10 | −0.82 | −0.58 | −1.28 | −0.95 | 0.11 | −8.86 | −0.60 | 0.25 | −3.96 |
| PDE1A | −1.21 | −1.30 | −0.76 | −0.53 | −0.95 | 0.11 | −8.88 | −1.19 | 0.06 | −15.53 |
| AW467174 | −1.37 | −1.23 | −0.73 | −0.49 | −0.95 | 0.11 | −8.95 | −0.94 | 0.11 | −8.72 |
| PLAT | −1.34 | −1.10 | −0.43 | −0.95 | −0.95 | 0.11 | −8.98 | −1.15 | 0.07 | −14.01 |
| LOC441047 | −1.28 | −1.19 | −0.77 | −0.58 | −0.95 | 0.11 | −9.01 | | | |
| CXCR4 | −1.59 | −1.32 | −0.53 | −0.41 | −0.96 | 0.11 | −9.16 | −1.21 | 0.06 | −16.40 |
| AK3L1 | −1.35 | −1.13 | −0.82 | −0.57 | −0.97 | 0.11 | −9.27 | −1.01 | 0.10 | −10.23 |
| SMPDL3A | −1.46 | −1.05 | −0.67 | −0.71 | −0.97 | 0.11 | −9.37 | −1.13 | 0.07 | −13.34 |
| KIAA0960 | −1.36 | −0.63 | −0.52 | −1.38 | −0.97 | 0.11 | −9.39 | −1.06 | 0.09 | −11.55 |
| THC2616558 | | | | | | | | −0.96 | 0.11 | −9.15 |
| LHFP | −1.26 | −1.02 | −0.97 | −0.66 | −0.98 | 0.11 | −9.52 | −0.99 | 0.10 | −9.78 |
| CPM | −1.17 | −0.96 | −0.92 | −0.86 | −0.98 | 0.11 | −9.52 | −0.85 | 0.14 | −7.05 |
| A_24_P345290 | −1.33 | −1.22 | −0.85 | −0.55 | −0.99 | 0.10 | −9.75 | −1.03 | 0.09 | −10.72 |
| PNOC | −1.04 | −1.15 | −0.92 | −0.91 | −1.01 | 0.10 | −10.18 | −0.78 | 0.16 | −6.08 |
| GALNT14 | −1.39 | −1.16 | −0.68 | −0.81 | −1.01 | 0.10 | −10.26 | −1.13 | 0.07 | −13.37 |
| TM4SF1 | −0.98 | −1.11 | −1.03 | −0.98 | −1.02 | 0.09 | −10.59 | −1.04 | 0.09 | −10.95 |
| ZAR1 | −1.08 | −0.98 | −1.03 | −1.04 | −1.03 | 0.09 | −10.75 | −0.93 | 0.12 | −8.53 |
| A_23_P10091 | −1.12 | −0.86 | −1.33 | −0.91 | −1.05 | 0.09 | −11.31 | | | |
| GLT8D2 | −1.28 | −1.21 | −0.92 | −0.84 | −1.06 | 0.09 | −11.53 | | | |
| RXFP1 | −1.81 | −1.24 | −0.71 | −0.55 | −1.08 | 0.08 | −11.90 | −1.02 | 0.10 | −10.51 |
| CGNL1 | −1.40 | −1.26 | −0.95 | −0.73 | −1.08 | 0.08 | −12.08 | −1.07 | 0.08 | −11.86 |
| AK094972 | −1.43 | −1.35 | −0.75 | −1.05 | −1.14 | 0.07 | −13.93 | −1.03 | 0.09 | −10.78 |
| LRCH2 | −1.27 | −1.00 | −1.15 | −1.34 | −1.19 | 0.06 | −15.54 | | | |
| BM930757 | −1.30 | −1.11 | −1.20 | −1.20 | −1.20 | 0.06 | −15.91 | | | |
| ATP8A1 | −1.34 | −1.38 | −1.15 | −0.98 | −1.21 | 0.06 | −16.34 | | | |
| SOX9 | −1.25 | −1.31 | −1.34 | −0.97 | −1.22 | 0.06 | −16.47 | −1.01 | 0.10 | −10.15 |
| SLC39A8 | −1.36 | −1.12 | −1.32 | −1.22 | −1.25 | 0.06 | −17.94 | −1.24 | 0.06 | −17.50 |
| TMEM47 | −1.59 | −1.10 | −1.13 | −1.29 | −1.28 | 0.05 | −18.94 | −1.44 | 0.04 | −27.45 |
| SLC10A4 | −1.23 | −1.12 | −1.28 | −1.51 | −1.29 | 0.05 | −19.33 | −1.21 | 0.06 | −16.04 |
| EDG7 | −1.35 | −1.32 | −1.27 | −1.36 | −1.33 | 0.05 | −21.23 | −1.31 | 0.05 | −20.52 |
| ITGA2 | −1.74 | −1.39 | −1.03 | −1.45 | −1.40 | 0.04 | −25.31 | −1.52 | 0.03 | −33.10 |
| SLC1A3 | −1.75 | −1.42 | −1.09 | −1.37 | −1.41 | 0.04 | −25.49 | −1.28 | 0.05 | −18.89 |
| PLCXD3 | −1.28 | −1.22 | −1.33 | −1.93 | −1.44 | 0.04 | −27.49 | −1.26 | 0.05 | −18.20 |
| BF514799 | −1.37 | −1.45 | −1.38 | −1.70 | −1.47 | 0.03 | −29.80 | | | |
| SLC16A12 | −1.56 | −1.34 | −1.54 | −1.50 | −1.48 | 0.03 | −30.49 | −1.43 | 0.04 | −26.86 |
| THC2208430 | −1.88 | −1.83 | −1.03 | −1.39 | −1.53 | 0.03 | −34.08 | −1.52 | 0.03 | −33.10 |
| THC2182743 | −1.60 | −1.49 | −1.47 | −1.67 | −1.56 | 0.03 | −36.19 | −1.38 | 0.04 | −24.21 |
| C4orf18 | −1.67 | −1.49 | −1.58 | −1.57 | −1.58 | 0.03 | −37.64 | −1.60 | 0.03 | −39.96 |
| ANKRD38 | −1.71 | −1.50 | −1.53 | −1.62 | −1.59 | 0.03 | −38.90 | −1.34 | 0.05 | −22.01 |
| CALCRL | −1.74 | −1.77 | −1.92 | −1.84 | −1.82 | 0.02 | −66.13 | | | |
| LOC152573/SHISA3 | −2.17 | −2.80 | −2.05 | −1.79 | −2.20 | 0.01 | −159.40 | −2.27 | 0.01 | −187.42 |

TABLE 5
List of the over- and under-expressed genes by at least two fold in the
docetaxel resistant LNCaP cell-lines at 0.5, 2.5, 5 and 12 nM.

| Primary | | | Log(Ratio) for each dose | | | | | | |
|---|---|---|---|---|---|---|---|---|---|
| Sequence | | | (nM) | | | | Log(Ratio) | Ratio | Fold Change |
| Name | Sequence Description | Accession# | 0.5 | 2.5 | 5 | 12 | Mean | Mean | Mean |
| PCDH7 | Homo sapiens protocadherin 7, transcript | NM_032456 | 0.51 | 1.18 | 1.41 | 1.35 | 1.11 | 12.97 | 12.97 |
| | variant b, | | | | | | | | |
| LPHN2 | Homo sapiens latrophilin 2 | NM_012302 | 0.51 | 0.85 | 1.35 | 1.52 | 1.06 | 11.39 | 11.39 |
| CBLN2 | Homo sapiens cerebellin 2 precursor | NM_182511 | 0.52 | 0.90 | 0.99 | 1.02 | 0.86 | 7.21 | 7.21 |
| FAM19A2 | Homo sapiens family with sequence | NM_178539 | 0.44 | 0.65 | 0.66 | 0.87 | 0.66 | 4.54 | 4.54 |
| | similarity 19 (chemokine (C-C motif)-like), | | | | | | | | |
| | member A2 | | | | | | | | |
| SESN3 | Homo sapiens sestrin 3 (SESN3), | NM_144665 | 0.32 | 1.07 | 0.48 | 0.50 | 0.59 | 3.92 | 3.92 |
| NEBL | Homo sapiens nebulette, transcript variant 1, | NM_006393 | 0.36 | 0.66 | 0.56 | 0.60 | 0.54 | 3.49 | 3.49 |
| ST6GAL1 | Homo sapiens ST6 beta-galactosamide | NM_173216 | 0.36 | 0.66 | 0.38 | 0.76 | 0.54 | 3.45 | 3.45 |
| | alpha-2,6-sialyltranferase 1, transcript | | | | | | | | |
| | variant 1, | | | | | | | | |
| GAGE1 | Homo sapiens G antigen 1, mRNA (cDNA | BC036094 | −0.35 | 0.55 | 0.91 | 0.86 | 0.49 | 3.12 | 3.12 |
| | clone MGC: 33825 IMAGE: 5296612), | | | | | | | | |
| | complete cds. | | | | | | | | |
| LIN7A | Homo sapiens lin-7 homolog A (C. elegans) | NM_004664 | 0.42 | 0.68 | 0.37 | 0.44 | 0.48 | 3.00 | 3.00 |
| ZMYND12 | Homo sapiens zinc finger, MYND-type | NM_032257 | 0.37 | 0.52 | 0.33 | 0.56 | 0.45 | 2.79 | 2.79 |
| | containing 12 | | | | | | | | |
| TCEA3 | Homo sapiens transcription elongation factor | NM_003196 | 0.30 | 0.57 | 0.44 | 0.41 | 0.43 | 2.70 | 2.70 |
| | A (SII), 3 | | | | | | | | |
| BBC3 | Homo sapiens BCL2 binding component 3 | NM_014417 | 0.34 | 0.61 | 0.35 | 0.41 | 0.43 | 2.67 | 2.67 |
| FAM8A1 | Homo sapiens family with sequence | NM_016255 | 0.43 | 0.65 | 0.30 | 0.33 | 0.43 | 2.67 | 2.67 |
| | similarity 8, member A1 | | | | | | | | |
| JAK3 | Homo sapiens Janus kinase 3 (a protein | BC028068 | 0.38 | 0.36 | 0.40 | 0.48 | 0.40 | 2.52 | 2.52 |
| | tyrosine kinase, leukocyte), mRNA (cDNA | | | | | | | | |
| | clone MGC: 39993 IMAGE: 5212575), | | | | | | | | |
| | complete cds. | | | | | | | | |
| ZSCAN18 | Homo sapiens zinc finger and SCAN domain | NM_023926 | 0.35 | 0.33 | 0.50 | 0.40 | 0.39 | 2.48 | 2.48 |
| | containing 18 | | | | | | | | |
| RPL31 | Homo sapiens ribosomal protein L31, | NM_001099693 | −0.32 | 0.41 | 0.40 | 0.57 | 0.26 | 1.83 | 1.83 |
| | transcript variant 4, | | | | | | | | |
| PVRL3 | Homo sapiens poliovirus receptor-related 3 | NM_015480 | −0.35 | 0.33 | 0.49 | 0.53 | 0.25 | 1.78 | 1.78 |
| ACOX2 | Homo sapiens acyl-Coenzyme A oxidase 2, | NM_003500 | 0.34 | 0.90 | −0.52 | −0.54 | 0.04 | 1.11 | 1.11 |
| | branched chain | | | | | | | | |
| FUT3 | Homo sapiens fucosyltransferase 3 | NM_000149 | 0.52 | 0.34 | −0.36 | −0.40 | 0.03 | 1.06 | 1.06 |
| | (galactoside 3(4)-L-fucosyltransferase, | | | | | | | | |
| | Lewis blood group), transcript variant 1, | | | | | | | | |
| B3GALT4 | Homo sapiens UDP-Gal:betaGlcNAc beta | NM_003782 | 0.40 | 0.36 | −0.39 | −0.55 | −0.03 | 0.94 | −1.06 |
| | 1,3-galactosyltransferase, polypeptide 4 | | | | | | | | |
| APOC1 | Homo sapiens apolipoprotein C-I | NM_001645 | 0.43 | 0.44 | −0.41 | −0.66 | −0.04 | 0.90 | −1.11 |
| RPRML | Homo sapiens reprimo-like | NM_203400 | 0.37 | 0.43 | −0.39 | −0.72 | −0.08 | 0.84 | −1.20 |
| BMP7 | Homo sapiens bone morphogenetic protein | NM_001719 | 0.36 | 0.47 | −0.53 | −0.68 | −0.09 | 0.80 | −1.24 |
| | 7 (osteogenic protein 1) | | | | | | | | |
| THC2645080 | Unknown | | −0.61 | −0.40 | −0.39 | 0.36 | −0.26 | 0.55 | −1.82 |
| KCND2 | Homo sapiens potassium voltage-gated | NM_012281 | 0.46 | −0.54 | −0.67 | −0.73 | −0.37 | 0.43 | −2.35 |
| | channel, Shal-related subfamily, member 2 | | | | | | | | |
| ASRGL1 | Homo sapiens asparaginase like 1, | NM_001083926 | 0.32 | −0.44 | −0.55 | −0.82 | −0.37 | 0.42 | −2.35 |
| | transcript variant 1, | | | | | | | | |
| AK026984 | Homo sapiens cDNA: FLJ23331 fis, clone | AK026984 | −0.34 | −0.59 | −0.38 | −0.38 | −0.42 | 0.38 | −2.65 |
| | HEP12664. | | | | | | | | |
| THC2550202 | Q96HL9_HUMAN (Q96HL9) CHP protein, | BF977562 | −0.47 | −0.32 | −0.49 | −0.41 | −0.42 | 0.38 | −2.65 |
| | partial (59%) | | | | | | | | |
| GMNN | Homo sapiens geminin, DNA replication | NM_015895 | −0.33 | −0.36 | −0.43 | −0.61 | −0.43 | 0.37 | −2.70 |
| | inhibitor | | | | | | | | |
| ZNF107 | Homo sapiens zinc finger protein 107 | NM_016220 | −0.40 | −0.73 | −0.33 | −0.34 | −0.45 | 0.35 | −2.82 |
| | transcript variant 1, | | | | | | | | |
| CEP152 | Homo sapiens centrosomal protein 152 kDa | NM_014985 | −0.34 | −0.67 | −0.36 | −0.47 | −0.46 | 0.35 | −2.87 |
| CDC7 | Homo sapiens cell division cycle 7 homolog | NM_003503 | −0.32 | −0.60 | −0.34 | −0.58 | −0.46 | 0.35 | −2.89 |
| | (S. cerevisiae) | | | | | | | | |
| ZNF91 | Homo sapiens zinc finger protein 91 | NM_003430 | −0.42 | −0.63 | −0.37 | −0.42 | −0.46 | 0.35 | −2.89 |
| KRT80 | Homo sapiens keratin 80, transcript variant | NM_182507 | −0.36 | −0.42 | −0.39 | −0.70 | −0.47 | 0.34 | −2.95 |
| | 1, | | | | | | | | |
| ANK3 | Homo sapiens cDNA FLJ44903 fis, clone | AK126851 | −0.63 | −0.64 | −0.33 | −0.31 | −0.48 | 0.33 | −3.01 |
| | BRAMY3005184, highly similar to Mus | | | | | | | | |
| | musculus ankyrin 3, epithelial. | | | | | | | | |
| THOC2 | Homo sapiens THO complex 2, transcript | NM_001081550 | −0.33 | −0.85 | −0.42 | −0.32 | −0.48 | 0.33 | −3.03 |
| | variant 1, | | | | | | | | |
| THC2633747 | ALU2_HUMAN (P39189) Alu subfamily SB | | −0.49 | −0.49 | −0.63 | −0.32 | −0.48 | 0.33 | −3.04 |
| | sequence contamination warning entry, | | | | | | | | |
| | partial (3%) | | | | | | | | |
| DEPDC1 | Homo sapiens DEP domain containing 1 | NM_017779 | −0.39 | −0.61 | −0.42 | −0.67 | −0.52 | 0.30 | −3.34 |
| | transcript variant 2, | | | | | | | | |
| AKAP9 | Homo sapiens A kinase (PRKA) anchor | NM_147171 | −0.40 | −0.77 | −0.59 | −0.34 | −0.52 | 0.30 | −3.34 |
| | protein (yotiao) 9, transcript variant 1, | | | | | | | | |
| GULP1 | Homo sapiens GULP, engulfment adaptor | NM_016315 | −0.53 | −0.66 | −0.59 | −0.35 | −0.53 | 0.29 | −3.42 |
| | PTB domain containing 1, | | | | | | | | |
| BRCA2 | Homo sapiens breast cancer 2, early onset, | NM_000059 | −0.53 | −0.76 | −0.37 | −0.49 | −0.54 | 0.29 | −3.45 |
| CYorf15B | lipopolysaccaride-specific response 5-like | AF332225 | −0.33 | −0.80 | −0.58 | −0.45 | −0.54 | 0.29 | −3.47 |
| | protein | | | | | | | | |
| | [Source: RefSeq_peptide; Acc: NP_115965] | | | | | | | | |
| | [ENST00000382832] | | | | | | | | |
| C6orf167 | Homo sapiens chromosome 6 open reading | NM_198468 | −0.51 | −0.96 | −0.31 | −0.39 | −0.54 | 0.29 | −3.48 |
| | frame 167, | | | | | | | | |
| RASSF8 | Homo sapiens clone B4-E11 carcinoma | AY665468 | −0.30 | −0.94 | −0.65 | −0.33 | −0.56 | 0.28 | −3.60 |
| | associated protein HOJ-1 mRNA, complete | | | | | | | | |
| | cds, alternatively spliced. | | | | | | | | |
| AL050204 | Homo sapiens mRNA; cDNA | AL050204 | −0.45 | −0.82 | −0.56 | −0.43 | −0.57 | 0.27 | −3.69 |
| | DKFZp586F1223 (from clone | | | | | | | | |
| | DKFZp586F1223). | | | | | | | | |
| THC2619021 | CF066_HUMAN (Q9P032) UPF0240 protein | | −0.35 | −1.04 | −0.55 | −0.39 | −0.58 | 0.26 | −3.80 |
| | C6orf66, complete | | | | | | | | |
| SGEF | Homo sapiens Src homology 3 domain- | NM_015595 | −0.48 | −0.76 | −0.59 | −0.52 | −0.59 | 0.26 | −3.85 |
| | containing guanine nucleotide exchange | | | | | | | | |
| | factor | | | | | | | | |
| NAB1 | Homo sapiens NGFI-A binding protein 1 | NM_005966 | −0.31 | −1.05 | −0.54 | −0.49 | −0.60 | 0.25 | −3.95 |
| | (EGR1 binding protein 1) | | | | | | | | |
| THC2645336 | Unknown | | −0.39 | −0.58 | −0.74 | −0.71 | −0.61 | 0.25 | −4.03 |
| THC2668270 | Q8I727_TRYCR (Q8I727) TcC31.32, partial | | −0.35 | −1.06 | −0.59 | −0.44 | −0.61 | 0.25 | −4.05 |
| | (14%) | | | | | | | | |
| CDK6 | Homo sapiens cyclin-dependent kinase 6 | NM_001259 | −0.50 | −0.94 | −0.68 | −0.36 | −0.62 | 0.24 | −4.20 |
| FAM59A | Homo sapiens family with sequence | NM_022751 | −0.41 | −0.74 | −0.61 | −0.77 | −0.63 | 0.23 | −4.28 |
| | similarity 59, member A | | | | | | | | |
| MPHOSPH1 | Homo sapiens M-phase phosphoprotein 1 | NM_016195 | −0.55 | −0.88 | −0.47 | −0.65 | −0.64 | 0.23 | −4.37 |
| BIRC3 | Homo sapiens baculoviral IAP repeat- | NM_001165 | −0.69 | −0.68 | −0.72 | −0.48 | −0.64 | 0.23 | −4.38 |
| | containing 3 (BIRC3), transcript variant 1, | | | | | | | | |
| ADAM22 | ADAM 22 precursor (A disintegrin and | AL133090 | −0.42 | −0.99 | −0.69 | −0.50 | −0.65 | 0.22 | −4.45 |
| | metalloproteinase domain 22) | | | | | | | | |
| | (Metalloproteinase-like, disintegrin-like, and | | | | | | | | |
| | cysteine-rich protein 2) (Metalloproteinase- | | | | | | | | |
| | disintegrin ADAM22-3). | | | | | | | | |
| | [Source: Uniprot/SWISSPROT; Acc: Q9P0K1] | | | | | | | | |
| | [ENST00000398204] | | | | | | | | |
| THC2733296 | Unknown | | −0.68 | −0.72 | −0.85 | −0.38 | −0.66 | 0.22 | −4.55 |
| AK090762 | Homo sapiens cDNA FLJ33443 fis, clone | AK090762 | −0.80 | −0.99 | −0.41 | −0.46 | −0.66 | 0.22 | −4.62 |
| | BRALZ1000103. | | | | | | | | |
| GBP1 | Homo sapiens guanylate binding protein 1, | NM_002053 | −0.43 | −0.47 | −0.87 | −0.96 | −0.68 | 0.21 | −4.80 |
| | interferon-inducible, 67 kDa | | | | | | | | |
| AK022086 | Homo sapiens cDNA FLJ12024 fis, clone | AK022086 | −0.81 | −0.88 | −0.76 | −0.33 | −0.70 | 0.20 | −4.97 |
| | HEMBB1001797. | | | | | | | | |
| BF831953 | PM3-HT0909-181000-010-b11 HT0909 | BF831953 | −0.71 | −0.87 | −0.84 | −0.39 | −0.71 | 0.20 | −5.09 |
| | Homo sapiens cDNA. | | | | | | | | |
| TUBB6 | Homo sapiens tubulin, beta 6 | NM_032525 | 0.40 | −0.47 | −1.28 | −1.49 | −0.71 | 0.20 | −5.11 |
| THC2677659 | Unknown | | −0.74 | −0.94 | −0.76 | −0.47 | −0.73 | 0.19 | −5.37 |
| ZNF93 | Homo sapiens cDNA FLJ39023 fis, clone | AK096342 | −0.77 | −0.88 | −0.65 | −0.64 | −0.74 | 0.18 | −5.45 |
| | NT2RP7004348, highly similar to ZINC | | | | | | | | |
| | FINGER PROTEIN 93. | | | | | | | | |
| PCGF5 | Homo sapiens polycomb group ring finger 5 | NM_032373 | −0.58 | −1.15 | −0.76 | −0.48 | −0.74 | 0.18 | −5.50 |
| THC2669975 | Unknown | | −0.91 | −0.89 | −0.78 | −0.38 | −0.74 | 0.18 | −5.53 |
| ACOT9 | Homo sapiens acyl-CoA thioesterase 9, | NM_001037171 | 0.36 | −0.80 | −1.21 | −1.37 | −0.76 | 0.18 | −5.70 |
| | transcript variant 1, | | | | | | | | |
| TFF1 | Homo sapiens trefoil factor 1 | NM_003225 | −0.96 | −0.83 | −0.60 | −0.66 | −0.76 | 0.17 | −5.78 |
| SLITRK6 | Homo sapiens SLIT and NTRK-like family. | NM_032229 | −0.46 | −0.85 | −0.91 | −0.96 | −0.80 | 0.16 | −6.24 |
| | member 6 | | | | | | | | |
| THC2618142 | Unknown | | −0.30 | −1.21 | −0.85 | −0.85 | −0.81 | 0.16 | −6.40 |
| LOC389023 | Homo sapiens hypothetical gene supported | BC032913 | −0.92 | −0.82 | −1.03 | −1.18 | −0.99 | 0.10 | −9.77 |
| | by BC032913; BC048425, mRNA (cDNA | | | | | | | | |
| | clone IMAGE: 5265535). | | | | | | | | |
| WNT5A | Homo sapiens wingless-type MMTV | NM_003392 | −0.62 | −1.06 | −1.40 | −1.36 | −1.11 | 0.08 | −12.96 |
| | integration site family, member 5A | | | | | | | | |
| ADD3 | Homo sapiens adducin 3 (gamma), | NM_016824 | −0.40 | −1.39 | −1.42 | −1.60 | −1.20 | 0.06 | −15.99 |
| | transcript variant 1 | | | | | | | | |

TABLE 6
List of the over- and under-expressed genes by at least two fold in the
docetaxel resistant cell-lines IGR-CaP1 and LNCaP.

| | IGR- | IGR- | IGR- | IGR- | IGR- | IGR- | | | |
|---|---|---|---|---|---|---|---|---|---|
| | CaP1 | CaP1 | CaP1 | CaP1 | CaP1 | CaP1 | LNCaP | | |
| | 200 nM | 100 nM | 50 nM | 25 nM | 12 nM | 5 nM | 12 nM | LNCaP | Fold |
| Primary Sequence | Fold | Fold | Fold | Fold | Fold | Fold | Fold | 5 nM | Change |
| Name | Change | Change | Change | Change | Change | Change | Change | Fold Change | Means |
| RPIB9 | 53.22 | 34.75 | 30.56 | 32.60 | 18.03 | 39.18 | 3.48 | 6.94 | 27.35 |
| MID1 | 9.04 | 7.06 | 3.96 | 5.55 | 12.11 | 4.12 | 4.71 | 3.17 | 6.21 |
| AQP1 | 5.56 | 5.08 | 7.98 | 7.39 | 9.35 | 7.94 | 2.46 | 2.74 | 6.06 |
| TMSB4X | 2.54 | 2.75 | 2.42 | 2.62 | 3.39 | 3.04 | 14.12 | 5.22 | 4.51 |
| PDE4B | 3.53 | 2.98 | 6.92 | 4.12 | 3.12 | 2.68 | 7.19 | 5.54 | 4.51 |
| CDKN1C | 4.79 | 2.76 | 4.83 | 6.56 | 6.26 | 5.27 | 2.62 | 2.24 | 4.42 |
| ST6GAL1 | 2.36 | 2.80 | 6.02 | 8.07 | 3.18 | 2.10 | 5.71 | 2.39 | 4.08 |
| SUSD4 | 5.02 | 4.44 | 4.85 | 6.13 | 2.72 | 2.86 | 3.19 | 3.29 | 4.06 |
| AGT | 2.91 | 2.16 | 2.69 | 2.40 | 2.05 | 4.80 | 3.47 | 2.70 | 2.90 |
| CD55 | 6.09 | 6.10 | 5.50 | 4.61 | 2.02 | 2.45 | 3.09 | −6.81 | 2.88 |
| NRL | 2.64 | 2.86 | 3.04 | 3.23 | 4.30 | 2.60 | 2.21 | 2.08 | 2.87 |
| THC2665111 | −4.25 | −6.83 | −5.47 | −6.13 | −3.73 | −2.08 | −4.04 | −2.05 | −4.32 |
| UGT8 | −5.00 | −7.13 | −6.07 | −2.76 | −4.02 | −4.22 | −13.79 | −11.67 | −6.83 |
| MYB | −6.57 | −5.04 | −7.78 | −6.50 | −6.62 | −11.29 | −12.54 | −2.80 | −7.39 |
| GALNT14 | −15.17 | −18.92 | −24.68 | −14.50 | −8.55 | −6.51 | −12.22 | −5.63 | −13.27 |
| SMAD9 | −6.14 | −10.35 | −23.21 | −28.27 | −11.55 | −49.90 | −2.49 | −3.04 | −16.87 |
| DNAJC15 | −4.45 | −4.17 | −6.06 | −3.73 | −2.13 | −2.33 | −115.05 | −75.51 | −26.68 |
| LOC152573/SHISA3 | −403.46 | −649.23 | −148.25 | −636.50 | −28.66 | −61.18 | −4.48 | −3.08 | −241.86 |

TABLE 7
List of the over- and under-expressed genes by at
least two fold in all the docetaxel resistant cell-
lines IGR-CaP1 and in the two clones 3A11 and 3B1.

| | IGR-CaP1-R | 3A11 | 3B1 |
|---|---|---|---|
| Primary Sequence Name | Fold Change | Fold Change | Fold Change |
| CDH16 | 8.71 | 5.43 | 4.07 |
| AQP1 | 7.06 | 3.02 | 3.32 |
| PLEKHH2 | 6.43 | 2.44 | 2.60 |
| SFRP1 | 5.99 | 2.48 | 2.52 |
| C1orf88 | 5.41 | 2.03 | 3.28 |
| AL133090 | 5.23 | 3.32 | 3.19 |
| CDKN1C | 4.90 | 3.17 | 3.04 |
| IGF2 | 4.21 | 3.81 | 4.51 |
| CCPG1 | 4.00 | 4.28 | 4.34 |
| KIAA1505 | 3.99 | 5.25 | 4.19 |
| COL16A1 | 3.76 | 3.30 | 4.04 |
| LOC63920 | 3.50 | 2.25 | 2.53 |
| PTGES | −3.51 | −2.87 | −5.45 |
| BDNF | −3.58 | −4.49 | −3.63 |
| THC2668815 | −3.65 | −2.73 | −5.75 |
| MFHAS1 | −3.66 | −2.11 | −2.69 |
| LCP1 | −3.96 | −4.76 | −3.99 |
| C12orf59 | −5.12 | −2.26 | −2.69 |
| FGFBP1 | −5.18 | −2.50 | −7.64 |
| EPAS1 | −5.33 | −4.25 | −2.82 |
| SYTL3 | −5.97 | −4.86 | −5.83 |
| LZTS1 | −6.01 | −2.06 | −7.11 |
| OLR1 | −7.74 | −7.86 | −6.77 |
| PDE1A | −15.53 | −10.24 | −7.58 |
| PLCXD3 | −18.20 | −2.40 | −27.05 |
| ANKRD38 | −22.01 | −3.21 | −5.64 |
| THC2182743 | −24.21 | −3.86 | −3.97 |

1-19. (canceled)

20. An in vitro method for predicting or monitoring whether a patient affected by a cancer is responsive to a treatment with a molecule of the taxoid family, wherein the method comprises: 1) providing a biological sample from said subject; 2) determining in the biological sample the expression level of at least 5 genes selected from the group consisting of the genes listed in Tables 1, 1 bis, 2 and 2 bis and comparing the expression level of said at least 5 genes to a reference expression level, the reference expression level being the expression level of the genes in cell-lines or patients sensitive to the treatment by the molecule of the taxoid family wherein the over-expression of genes from Tables 1 and 1 bis and/or the under-expression of genes from Tables 2 and 2 bis are indicative of a resistance to the treatment by the molecule of the taxoid family and thereby predicting or monitoring whether a patient affected by a cancer is responsive to a treatment with a molecule of the taxoid family.

21. The method according to claim 20, wherein said at least 5 genes are selected from the group consisting of RPIB9, GALNT14, LZTS1, SHISA3, AQP1, CXCL2, AL137761, TFPI2, TNF, ABCB1, PURG, MCTP1, OAS3, GAS1, BIRC3, BQ186674, UBXD3, SFRP1, PLEKHH2, GNG11, CDH16, AKR1C1, MGC42367, RAFTLIN, FAM111A, ADAMTS1, FHOD3, DUSP23, ITGB8, IL15, IGFBP3, PHLDA1, GPC3, CACNG6, AKR1C3, C1orf88, CDKN1C, THC2753543, NTN4, MID1, CSPG2, AL133090, ST6GAL1, TMEFF1, FBXL16, CART1, C9orf150, HDAC9, GLS, CNTNAP3, PDE4B, DKFZp586I1420, ZNRF2, SP5, LAMA2, CD55, MANEAL, AK026140, KIAA1505, DEPDC6, PPP2R2C, ARHGDIB, RAI2, TXNRD3, ABCB2, RASSF8, CR622072, LITAF, IGF2, LOC389722, ANKRD18A, GRB10, AY336981, SLPI, COL16A1, GRAMD3, FAM107B, LOC440934, VCX, LAMB3, WNT5A, JAG1, NRL, AGT, TMSB4X, CCPG1, ADRA2C, TEX15, SEMA3B, NFKBIZ, AK096677, PTPRM, NQO1, AK022020, MGAT4A, LOC63920, AL390181, AK123483, FAM80A, PSCDBP, CKB, SLC7A8, PDK1, GATA2, PDLIM5, FLJ10159, PTGES, DNAJC15, NPAS1, THC2668815, TFDP2, PFKFB4, ENC1, NRP2, MFHAS1, AK024680, AL137342, D4S234E, LCP1, A_{—}32_P95067, THC2038567, BDNF, AW205591, AKAP12, NMNAT2, SLC12A3, SLC22A2, ANKRD37, LIN7A, PHEX, C1QL1, EPAS1, KCNC4, FGFBP1, SYTL3, HSHPX5, MGST1, THC2050576, SLC3A1, UGT8, SUNC1, DUSP13, AUTS2, PLAC8, MSX2, SMAD9, TTN, LRRN6C, MEIS2, DHRS3, OLR1, MOXD1, DCAMKL1, C12orf59, SALL1, FZD8, FLJ39502, PROS1, MYB, SLC16A10, GJA7, GAL, PLXNA2, PDE1A, AW467174, PLAT, CXCR4, AK3L1, SMPDL3A, KIAA0960, LHFP, CPM, A_{—}24_P345290, PNOC, TM4SF1, ZAR1, RXFP1, CGNL1, AK094972, SOX9, SLC39A8, TMEM47, SLC10A4, EDG7, ITGA2, SLC1A3, PLCXD3, BF514799, SLC16A12, THC2182743, C4orf18 and ANKRD38.

22. The method according to claim 20, wherein said at least 5 genes are selected from one of the following groups or a combination thereof:
a) RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, WNT2B, GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7,ITGA2, PLCXD3, BF514799, SLC16A12, THC2208430, THC2182743, C4orf18, ANKRD38, CALCRL, and LOC152573;RPIB9, CXCL2, TFPI2, TNF, ABCB 1, ADAMTS5, PURG, OAS3, GAS 1, BIRC3, MAL, GALNT14, TM4SF1, RXFP1, ATP8A1, SOX9, SLC39A8, EDG7, ITGA2, SLC1A3, CALCRL and LOC152573;RPIB9, CXCL2, AL137761, TFPI2, TNF, ABCB1, PURG, MCTP1, OAS3, GAS1, BIRC3, BQ186674, UBXD3, GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC152573/SHISA3; or RPIB9, CXCL2, TFPI2, TNF, ABCB1, PURG, OAS3, GAS1, BIRC3, GALNT14, TM4SF1, RXFP1, SOX9, SLC39A8, SLC1A3, EDG7, ITGA2, and LOC 152573/SHISA3;
b) RPIB9, TFPI2, ABCB1, BIRC3, WNT2B, SFRP1, FSTL1, AHR, CDKN1C, ABCB2, CYR61, WNT5A, ABCC3, JAG1, STAT1, WNT7B, CASP8, LZTS1, FZD8, GALNT14, RXFP1 and LOC152573;
c) TFPI2, AL137761, RPIB9, PURG, ABCB1, BIRC3, CXCL2, TNF, MCTP1, FAM111A, OAS3, ITGA2, TMEM47, SLC16A12, THC2182743, ANKRD38, SLC1A3, SLC39A8, CXCR4, PDE1A, LOC152573/SHISA3, BF514799, GALNT14 and PLAT;
d) RPIB9, MID1, AQP1, TMSB4X, PDE4B, CDKN1C, ST6GAL1, SUSD4, AGT, CD55, NRL, THC2665111, UGT8, MYB, GALNT14, SMAD9, DNAJC15, and SHISA3;
e) CDH16, AQP1, PLEKHH2, SFRP1, C1orf88, AL133090, CDKN1C, IGF2, CCPG1, KIAA1505, COL16A1, LOC63920, PTGES, BDNF, THC2668815, MFHAS1, LCP1, C12orf59, FGFBP1, EPASI, SYTL3, LZTS1, OLR1, PDE1A, PLCXD3, ANKRD38, and THC2182743;
f) ABCC3, CD55, COL16A1, DHRS3, FSTL1, GLS, HDL, HIVEPI, LAMA2, LAMB3, LIPG, LITAF, MAL, MFHAS1,NFKBIZ,NRP1, NRP2, OAS3, OLR1, PSCDBP, RFTN1, SCARB1, SEMA3B, SEMA3C, SFRP1, SLC1A3, ST6GAL1, TLR3, TM4SF1 and TNF;
g) ADAMTS1, ADRA2C, AKAP12, CDKN1C, CYR61, FBN1, GAS1, GPC3, IGF2, IGFBP3, JAG1, MGST1, NTN4, PDE1A, PDE4B, PDE4D, PDE4DIP, PDGFB, PHLDA1, PIM1, PPP2R2C, RGS16, SCD, SLC1A1, SMPDL3A, TFPI2 and VCAN;
h) ABCB1, AHR, AHRR, AMPH, BIRC3, CXCL2, CYP1A1, IL1R1, NQO1, PLAT, PLXNA2, SLC16A10, SLC3A1, SLC7A8, SLPI, TAP1, UGT8, UGT2B4, UGT2B7, UGT2B10, UGT2B11 and UGT2B28;
i) AQP1, ARHGDIB, BAMBI, CREB5, CXCR4, EPAS1, FGF2, FGFBP1, GRB10, IL15, MT2A, NUPR1, PDK1, PROS 1, PTPN3, RPS6KA2, TFDP2, WNT2B, WNT5A and WNT7B;
j) AGT, ATP8A2, BDNF, EDGE, GAL, GATA2, ITGA2, LRP11, LZTS1, MYB, NCALD, PNOC, PTGES, SRGAP3, TAC3 and TTN;
k) AFF1, ASGR1, BLVRA, CASP8, CD40, KCNH2, NRG1, NRL, PHEX, PLAC8, SMAD7, SMAD9, SOX9, SPG20 and STAT1;
l) TNF, ABCB1, CYP1A1, AHRR, AHR, PP2R2C, ABCC3, NQO1, PIK3C3, UGT2B7, UGT2B11, UGT2B28, UGT2B4, UGT2B10, CHST7, MGST1 and UGT8;
m) Wnt2B, Wnt5A, Wnt7B, SFRP1, FSTL1, Jag1, Cyr61, LOC152573, FZD8 and FOXL2;
n) ADAMRS1, COL16A1, PSCDBP, DHRS3, GAS 1, GLS, GPC3, IGF2, IGFBP3, LAMA2, LAMB3, LITAF, MFHAS1, MGST1, NFKBIZ, OAS3, OLR1, PHLDA1, PLAT, PNOC, RAFTLIN, RXFP1, SFRP1, SLC1A3, SLPI, ST6GAL1, TFPI2, TM4SF1, TNF, CSPG2 and WNT5A;
o) ABCB1, ADRA2C, AHR, AKAP12, BIRC3, CD44, CDH16, CDKN1C, CXCL2, EPAS1, HDAC9, MYB, PLXNA2, PTPRM, ROBO3, SLC16A10, SLC3A1, SLC7A8, ABCB2, TFDP2 and TNFSF13;
p) AQP1, GALNT14, ITGA2, ITGB8, NMNAT2, NPAS1, PDLIM5, SEMA3B, SLC12A3, SLC39A8, KIAA0960, TXNRD3, CSPG2 and ZNRF2;
q) CART1, CKB, EBF3, KRT7, LCP1, LRRN6C, THC2182743, MEIS2, NRP2, PROS1, RPIB9, SMPDL3A, UBXD3 and UGT8;
r) AKR1C1, C1QL1, CCPG1, D4S234E, DUSP23, FAM111A, FBXL16, GAL, MGAT4A, MID1, FAM80A and TMSB4X;
s) PCDH7, LPHN2, CBLN2, FAM19A2, SESN3, NEBL, ST6GAL1, LIN7A, ZMYND12, TCEA3, ADD3, WNT54, TFF1, ACOT9, PCGF5, TUBB6, GBP1, BIRC3, KIF208 and FAM59A;
t) JAK3, ADD3, AKAP9, B3GALT4, BRCA2, CDK6, DEPDC1, GMNN, GULP1, NDUFAF4, PCGF5, SESN3, TUBB6, ZNF91 and WNT5A; and
u) ACOT9, FUT3, LIN7A, NEBL, PCDH7, ST6GAL1, ASRGL1, BIRC3, BMP7, GBP1, KCND2, KIF20B and NAB1.

23. The method according to claim 20, wherein said molecule of the taxoid family is docetaxel, larotaxel, XRP6258, BMS-184476, BMS-188797, BMS-275183, ortataxel, RPR 109881A, RPR 116258, NBT-287, PG-paclitaxel, ABRAXANE®, Tesetaxel, IDN 5390, Taxoprexin, DHA-paclitaxel, or MAC-321.

24. The method according to claim 20, wherein the method comprises determining the expression level of at least 10, 15, 20, 25, 30, 40, 50, 60, 70, 80, 90, 100, 150, 200 or 300 genes from those listed in Tables 1, 1bis, 2 and 2bis.

25. The method according to claim 20, wherein the expression level of genes is determined by the quantity of protein or mRNA encoded by said genes.

26. The method according to claim 20, wherein the biological sample is a cancer sample.

27. The method according to claim 20, wherein the cancer is selected from the group consisting of breast cancer, lung cancer, prostate cancer, gastric cancer and head and neck cancer.

28. A kit for predicting or monitoring whether a patient affected by a cancer is responsive to a treatment with a molecule of the taxoid family, wherein the kit comprises detection means selected from the group consisting primer pairs, probes and antibodiesspecific to at least 5 genes selected from the group consisting of the genes listed in Tables 1, 1bis, 2, 2bis and 5-7.

29. The kit according to claim 28, wherein said at least 5 genes are selected from the group consisting of RPIB9, GALNT14, LZTS1, SHISA3, AQP1, CXCL2, AL137761, TFPI2, TNF, ABCB1, PURG, MCTP1, OAS3, GAS1, BIRC3, BQ186674, UBXD3, SFRP1, PLEKHH2, GNG11, CDH16, AKR1C1, MGC42367, RAFTLIN, FAM111A, ADAMTS1, FHOD3, DUSP23, ITGB8, IL15, IGFBP3, PHLDA1, GPC3, CACNG6, AKR1C3, C1orf88, CDKN1C, THC2753543, NTN4, MID1, CSPG2, AL133090, ST6GAL1, TMEFF1, FBXL16, CART1, C9orf150, HDAC9, GLS, CNTNAP3, PDE4B, DKFZp586I1420, ZNRF2, SP5, LAMA2, CD55, MANEAL, AK026140, KIAA1505, DEPDC6, PPP2R2C, ARHGDIB, RAI2, TXNRD3, ABCB2, RASSF8, CR622072, LITAF, IGF2, LOC389722, ANKRD18A, GRB10, AY336981, SLPI, COL16A1, GRAMD3, FAM107B, LOC440934, VCX, LAMB3, WNT5A, JAG1, NRL, AGT, TMSB4X, CCPG1, ADRA2C, TEX15, SEMA3B, NFKBIZ, AK096677, PTPRM, NQO1, AK022020, MGAT4A, LOC63920, AL390181, AK123483, FAM80A, PSCDBP, CKB, SLC7A8, PDK1, GATA2, PDLIM5, FLJ 10159, PTGES, DNAJC 15, NPAS1, THC2668815, TFDP2, PFKFB4, ENC1, NRP2, MFHAS1, AK024680, AL137342, D4S234E, LCP1, A_{—}32_P95067, THC2038567, BDNF, AW205591, AKAP12, NMNAT2, SLC12A3, SLC22A2, ANKRD37, LIN7A, PHEX, C1QL1, EPAS1, KCNC4, FGFBP1, SYTL3, HSHPX5, MGST1, THC2050576, SLC3A1, UGT8, SUNC1, DUSP13, AUTS2, PLAC8, MSX2, SMAD9, TTN, LRRN6C, MEIS2, DHRS3, OLR1, MOXD1, DCAMKL1, C12orf59, SALL1, FZD8, FLJ39502, PROS1, MYB, SLC16A10, GJA7, GAL, PLXNA2, PDE1A, AW467174, PLAT, CXCR4, AK3L1, SMPDL3A, KIAA0960, LHFP, CPM, A_{—}24_P345290, PNOC, TM4SF1, ZAR1, RXFP1, CGNL1, AK094972, SOX9, SLC39A8, TMEM47, SLC10A4, EDG7, ITGA2, SLC1A3, PLCXD3, BF514799, SLC16A12, THC2182743, C4orf18 and ANKRD38.

30. The kit according to claim 28, wherein said at least 5 genes are selected from one of the following groups or a combination thereof:
a) RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, WNT2B, GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7,ITGA2, PLCXD3, BF514799, SLC16A12, THC2208430, THC2182743, C4orf18, ANKRD38, CALCRL, and LOC152573; RPIB9, CXCL2, TFPI2, TNF, ABCB1, ADAMTS5, PURG, OAS3, GAS1, BIRC3, MAL, GALNT14, TM4SF1, RXFP1, ATP8A1, SOX9, SLC39A8, EDG7, ITGA2, SLC1A3, CALCRL and LOC152573; RPIB9, CXCL2, AL137761, TFPI2, TNF, ABCB1, PURG, MCTP1, OAS3, GAS1, BIRC3, BQ186674, UBXD3, GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC152573/SHISA3; orRPIB9, CXCL2, TFP12, TNF, ABCB1, PURG, OAS3, GAS1, BIRC3, GALNT14, TM4SF1, RXFP1, SOX9, SLC39A8, SLC1A3, EDG7, ITGA2, and LOC152573/SHISA3;
b) RPIB9, TFPI2, ABCB1, BIRC3, WNT2B, SFRP1, FSTL1, AHR, CDKN1C, ABCB2, CYR61, WNT5A, ABCC3, JAG1, STAT1, WNT7B, CASP8, LZTS1, FZD8, GALNT14, RXFP1 and LOC 152573;
c) TFPI2, AL137761, RPIB9, PURG, ABCB1, BIRC3, CXCL2, TNF, MCTP1, FAM111A, OAS3, ITGA2, TMEM47, SLC16A12, THC2182743, ANKRD38, SLC1A3, SLC39A8, CXCR4, PDE1A, LOC152573, BF514799, GALNT14 and PLAT;
d) RPIB9, MID1, AQP1, TMSB4X, PDE4B, CDKN1C, ST6GAL1, SUSD4, AGT, CD55, NRL, THC2665111, UGT8, MYB, GALNT14, SMAD9, DNAJC15, and SHISA3;
e) CDH16, AQP1, PLEKHH2, SFRP1, C1orf88, AL133090, CDKN1C, IGF2, CCPG1, KIAA1505, COL16A1, LOC63920, PTGES, BDNF, THC2668815, MFHAS1, LCP1, C12orf59, FGFBP1, EPAS1, SYTL3, LZTS1, OLR1, PDE1A, PLCXD3, ANKRD38, and THC2182743;
f) ABCC3, CD55, COL16A1, DHRS3, FSTL1, GLS, HDL, HIVEP1, LAMA2, LAMB3, LIPG, LITAF, MAL, MFHAS1, NFKBIZ, NRP1, NRP2, OAS3, OLR1, PSCDBP, RFTN1, SCARB1, SEMA3B, SEMA3C, SFRP1, SLC1A3, ST6GAL1, TLR3, TM4SF1 and TNF;
g) ADAMTS1, ADRA2C, AKAP12, CDKN1C, CYR61, FBN1, GAS1, GPC3, IGF2, IGFBP3, JAG1, MGST1, NTN4, PDE1A, PDE4B, PDE4D, PDE4DIP, PDGFB, PHLDA1, PIM1, PPP2R2C, RGS16, SCD, SLC1A1, SMPDL3A, TFPI2 and VCAN;
h) ABCB1, AHR, AHRR, AMPH, BIRC3, CXCL2, CYP1A1, IL1R1, NQO1, PLAT, PLXNA2, SLC16A10, SLC3A1, SLC7A8, SLPI, TAP1, UGT8, UGT2B4, UGT2B7, UGT2B10, UGT2B11 and UGT2B28;
i) AQP1, ARHGDIB, BAMBI, CREB5, CXCR4, EPAS1, FGF2, FGFBP1, GRB10, IL15, MT2A, NUPR1, PDK1, PROS1, PTPN3, RPS6KA2, TFDP2, WNT2B, WNT5A and WNT7B;
j) AGT, ATP8A2, BDNF, EDGE, GAL, GATA2, ITGA2, LRP11, LZTS1, MYB,NCALD, PNOC, PTGES, SRGAP3, TAC3 and TTN;
k) AFF1, ASGR1, BLVRA, CASP8, CD40, KCNH2, NRG1, NRL, PHEX, PLAC8, SMAD7, SMAD9, SOX9, SPG20 and STAT1;
l) TNF, ABCB1, CYP1A1, AHRR, AHR, PP2R2C, ABCC3, NQO1, PIK3C3, UGT2B7, UGT2B11, UGT2B28, UGT2B4, UGT2B10, CHST7, MGST1 and UGT8;
m) Wnt2B, Wnt5A, Wnt7B, SFRP1, FSTL1, Jag1, Cyr61, LOC152573, FZD8 and FOXL2;
n) ADAMRS1, COL16A1, PSCDBP, DHRS3, GAS1, GLS, GPC3, IGF2, IGFBP3, LAMA2, LAMB3, LITAF, MFHAS1, MGST1, NFKBIZ, OAS3, OLR1, PHLDA1, PLAT, PNOC, RAFTLIN, RXFP1, SFRP1, SLC1A3, SLPI, ST6GAL1, TFPI2, TM4SF1, TNF, CSPG2 and WNT5A;
o) ABCB1, ADRA2C, AHR, AKAP12, BIRC3, CD44, CDH16, CDKN1C, CXCL2, EPAS1, HDAC9, MYB, PLXNA2, PTPRM, ROBO3, SLC16A10, SLC3A1, SLC7A8, ABCB2, TFDP2 and TNFSF13;
p) AQP1, GALNT14, ITGA2, ITGB8, NMNAT2, NPAS1, PDLIM5, SEMA3B, SLC12A3, SLC39A8, KIAA0960, TXNRD3, CSPG2 and ZNRF2;
q) CART1, CKB, EBF3, KRT7, LCP1, LRRN6C, THC2182743, MEIS2, NRP2, PROS1, RPIB9, SMPDL3A, UBXD3 and UGT8;
r) AKR1C1, C1QL1, CCPG1, D4S234E, DUSP23, FAM111A, FBXL16, GAL, MGAT4A, MID1, FAM80A and TMSB4X;
s) PCDH7, LPHN2, CBLN2, FAM19A2, SESN3, NEBL, ST6GAL1, LIN7A, ZMYND12, TCEA3, ADD3, WNT54, TFF1, ACOT9, PCGF5, TUBB6, GBP1, BIRC3, KIF208 and FAM59A;
t) JAK3, ADD3, AKAP9, B3GALT4, BRCA2, CDK6, DEPDC1, GMNN, GULP1, NDUFAF4, PCGF5, SESN3, TUBB6, ZNF91 and WNT5A; and
u) ACOT9, FUT3, LIN7A, NEBL, PCDH7, ST6GAL1, ASRGL1, BIRC3, BMP7, GBP1, KCND2, KIF20B and NAB 1.

31. A DNA chip comprising a solid support coupled to nucleic acids that hybridize with at least 5 genes selected from the group consisting of the genes listed in Tables 1, 1bis, 2, 2bis and 5-7.

32. The DNA chip according to claim 31, wherein said at least 5 genes are selected from the group consisting of RPIB9, GALNT14, LZTS1, SHISA3, AQP1, CXCL2, AL137761, TFPI2, TNF, ABCB1, PURG, MCTP1, OAS3, GAS1, BIRC3, BQ186674, UBXD3, SFRP1, PLEKHH2, GNG11, CDH16, AKR1C1, MGC42367, RAFTLIN, FAM111A, ADAMTS1, FHOD3, DUSP23, ITGB8, IL15, IGFBP3, PHLDA1, GPC3, CACNG6, AKR1C3, C1orf88, CDKN1C, THC2753543, NTN4, MID1, CSPG2, AL133090, ST6GAL1, TMEFF1, FBXL16, CART1, C9orf150, HDAC9, GLS, CNTNAP3, PDE4B, DKFZp586I1420, ZNRF2, SP5, LAMA2, CD55, MANEAL, AK026140, KIAA1505, DEPDC6, PPP2R2C, ARHGDIB, RAI2, TXNRD3, ABCB2, RASSF8, CR622072, LITAF, IGF2, LOC389722, ANKRD18A, GRB10, AY336981, SLPI, COL16A1, GRAMD3, FAM107B, LOC440934, VCX, LAMB3, WNT5A, JAG1, NRL, AGT, TMSB4X, CCPGI, ADRA2C, TEX15, SEMA3B, NFKBIZ, AK096677, PTPRM, NQO1, AK022020, MGAT4A, LOC63920, AE390181, AK123483, FAM80A, PSCDBP, CKB, SLC7A8, PDK1, GATA2, PDLIM5, FLJ10159, PTGES, DNAJC15, NPAS1, THC2668815, TFDP2, PFKFB4, ENC1, NRP2, MFHAS 1, AK024680, AL137342, D4S234E, LCP1, A_{—}32_P95067, THC2038567, BDNF, AW205591, AKAP12, NMNAT2, SLC12A3, SLC22A2, ANKRD37, LIN7A, PHEX, C1QL1, EPAS1, KCNC4, FGFBP1, SYTL3, HSHPX5, MGST1, THC2050576, SLC3A1, UGT8, SUNC1, DUSP13, AUTS2, PLAC8, MSX2, SMAD9, TTN, LRRN6C, MEIS2, DHRS3, OLR1, MOXD1, DCAMKL1, C12orf59, SALL1, FZD8, FLJ39502, PROS1, MYB, SLC16A10, GJA7, GAL, PLXNA2, PDE1A, AW467174, PLAT, CXCR4, AK3L1, SMPDL3A, KIAA0960, LHFP, CPM, A_{—}24_P345290, PNOC, TM4SF1, ZAR1, RXFP1, CGNL1, AK094972, SOX9, SLC39A8, TMEM47, SLC10A4, EDG7, ITGA2, SLC1A3, PLCXD3, BF514799, SLC16A12, THC2182743, C4orf18 and ANKRD38.

33. The DNA chip according to claim 31, wherein said at least 5 genes are selected from one of the following groups or a combination thereof:
a) RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, WNT2B, GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7,ITGA2, PLCXD3, BF514799, SLC16A12, THC2208430, THC2182743, C4orf18, ANKRD38, CALCRL, and LOC152573; RPIB9, CXCL2, TFPI2, TNF, ABCB1, ADAMTS5, PURG, OAS3, GAS1, BIRC3, MAL, GALNT14, TM4SF1, RXFP1, ATP8A1, SOX9, SLC39A8, EDG7, ITGA2, SLC1A3, CALCRL and LOC152573; RPIB9, CXCL2, AL137761, TFPI2, TNF, ABCB1, PURG, MCTP1, OAS3, GAS1, BIRC3, BQ186674, UBXD3, GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC152573/SHISA3; orRPIB9, CXCL2, TFPI2, TNF, ABCB1, PURG, OAS3, GAS1, BIRC3, GALNT14, TM4SF1, RXFP1, SOX9, SLC39A8, SLCIA3, EDG7, ITGA2, and LOC152573/SHISA3;
b) RPIB9, TFPI2, ABCB1, BIRC3, WNT2B, SFRP1, FSTL1, AHR, CDKN1C, ABCB2, CYR61, WNT5A, ABCC3, JAG1, STAT1, WNT7B, CASP8, LZTS1, FZD8, GALNT14, RXFP1 and LOC152573;
c) TFPI2, AL137761, RPIB9, PURG, ABCB1, BIRC3, CXCL2, TNF, MCTP1, FAM111A, OAS3, ITGA2, TMEM47, SLC16A12, THC2182743, ANKRD38, SLC1A3, SLC39A8, CXCR4, PDE1A, LOC152573, BF514799, GALNT14 and PLAT;
d) RPIB9, MID1, AQP1, TMSB4X, PDE4B, CDKN1C, ST6GAL1, SUSD4, AGT, CD55, NRL, THC2665111, UGT8, MYB, GALNT14, SMAD9, DNAJC15, and SHISA3;
e) CDH16, AQP1, PLEKHH2, SFRP1, C1orf88, AL133090, CDKN1C, IGF2, CCPG1, KIAA1505, COL16A1, LOC63920, PTGES, BDNF, THC2668815, MFHAS1, LCP1, C12orf59, FGFBP1, EPAS1, SYTL3, LZTS1, OLR1, PDE1A, PLCXD3, ANKRD38, and THC2182743;
f) ABCC3, CD55, COL16A1, DHRS3, FSTL1, GLS, HDL, HIVEP1, LAMA2, LAMB3, LIPG, LITAF, MAL, MFHAS1,NFKBIZ,NRP1, NRP2, OAS3, OLR1, PSCDBP, RFTN1, SCARB1, SEMA3B, SEMA3C, SFRP1, SLCIA3, ST6GAL1, TLR3, TM4SF1 and TNF;
g) ADAMTS1, ADRA2C, AKAP12, CDKN1C, CYR61, FBN1, GAS1, GPC3, IGF2, IGFBP3, JAG1, MGST1, NTN4, PDE1A, PDE4B, PDE4D, PDE4DIP, PDGFB, PHLDA1, PIM1, PPP2R2C, RGS16, SCD, SLC1A1, SMPDL3A, TFPI2 and VCAN;
h) ABCB1, AHR, AHRR, AMPH, BIRC3, CXCL2, CYP1A1, IL1R1, NQO1, PLAT, PLXNA2, SLC16A10, SLC3A1, SLC7A8, SLPI, TAP1, UGT8, UGT2B4, UGT2B7, UGT2B10, UGT2B11 and UGT2B28;
i) AQP1, ARHGDIB, BAMBI, CREB5, CXCR4, EPAS1, FGF2, FGFBP1, GRB10, IL15, MT2A, NUPR1, PDK1, PROS1, PTPN3, RPS6KA2, TFDP2, WNT2B, WNT5A and WNT7B;
j) AGT, ATP8A2, BDNF, EDGE, GAL, GATA2, ITGA2, LRP11, LZTS1, MYB, NCALD, PNOC, PTGES, SRGAP3, TAC3 and TTN;
k) AFF1, ASGR1, BLVRA, CASP8, CD40, KCNH2, NRG1, NRL, PHEX, PLAC8, SMAD7, SMAD9, SOX9, SPG20 and STAT1;
l) TNF, ABCB1, CYP1A1, AHRR, AHR, PP2R2C, ABCC3, NQO1, PIK3C3, UGT2B7, UGT2B11, UGT2B28, UGT2B4, UGT2B10, CHST7, MGST1 and UGT8;
m) Wnt2B, Wnt5A, Wnt7B, SFRP1, FSTL1, Jag1, Cyr61, LOC152573, FZD8 and FOXL2;
n) ADAMRS1, COL16A1, PSCDBP, DHRS3, GAS1, GLS, GPC3, IGF2, IGFBP3, LAMA2, LAMB3, LITAF, MFHAS1, MGST1, NFKBIZ, OAS3, OLR1, PHLDA1, PLAT, PNOC, RAFTLIN, RXFP1, SFRP1, SLC1A3, SLPI, ST6GAL1, TFPI2, TM4SF1, TNF, CSPG2 and WNT5A;
o) ABCB1, ADRA2C, AHR, AKAP12, BIRC3, CD44, CDH16, CDKN1C, CXCL2, EPAS1, HDAC9, MYB, PLXNA2, PTPRM, ROBO3, SLC16A10, SLC3A1, SLC7A8, ABCB2, TFDP2 and TNFSF13;
p) AQP1, GALNT14, ITGA2, ITGB8, NMNAT2, NPAS1, PDLIM5, SEMA3B, SLC12A3, SLC39A8, KIAA0960, TXNRD3, CSPG2 and ZNRF2;
q) CART1, CKB, EBF3, KRT7, LCP1, LRRN6C, THC2182743, MEIS2, NRP2, PROS1, RPIB9, SMPDL3A, UBXD3 and UGT8;
r) AKR1C1, C1QL1, CCPG1, D4S234E, DUSP23, FAM111A, FBXL16, GAL, MGAT4A, MID1, FAM80A and TMSB4X;
s) PCDH7, LPHN2, CBLN2, FAM19A2, SESN3, NEBL, ST6GAL1, LIN7A, ZMYND12, TCEA3, ADD3, WNT54, TFF1, ACOT9, PCGF5, TUBB6, GBP1, BIRC3, KIF208 and FAM59A;
t) JAK3, ADD3, AKAP9, B3GALT4, BRCA2, CDK6, DEPDC1, GMNN, GULP1, NDUFAF4, PCGF5, SESN3, TUBB6, ZNF91 and WNT5A; and
u) ACOT9, FUT3, LIN7A, NEBL, PCDH7, ST6GAL1, ASRGL1, BIRC3, BMP7, GBP1, KCND2, KIF20B and NAB 1.

34. A method for screening or identifying a compound suitable for improving the treatment of a cancer with a molecule of the taxoid family or for reducing the resistance development during the treatment of a cancer with a molecule of the taxoid family, comprising 1) providing a cell-line with at least 5 genes over-expressed and/or under-expressed respectively selected from the group of over-expressed genes of Tables 1, 1bis and over-expressed genes of Tables 5-7 and under-expressed genes of Tables 2, 2bis and under-expressed genes of Tables 5-7; 2) contacting said cell-line with a test compound; 3) determining the expression level of said at least 5 genes; and 4) selecting the compound which decreases the expression level of the over-expressed genes and increases the expression level of the under-expressed genes.

35. A method for screening or identifying a compound suitable for improving the treatment of a cancer with a molecule of the taxoid family or for reducing the resistance development during the treatment of a cancer with the molecule of the taxoid family, comprising 1) providing a cell-line sensitive to the molecule of the taxoid family; 2) contacting said cell-line with a test compound and the molecule of the taxoid family; 3) determining the expression level of said at least 5 genes selected from the genes listed in Tables 1, 1bis, 2 and 2bis; and 4) selecting the compound which inhibits the appearance of an over-expression and/or an under-expression of at least 5 genes respectively selected from the group of genes of Tables 1 and ibis and genes of Tables 2 and 2bis.

36. A method for screening or identifying a compound suitable for improving the treatment of a cancer with a molecule of the taxoid family or for reducing the resistance development during the treatment of a cancer with the molecule of the taxoid family, comprising 1) providing a cell-line with at least on gene over-expressed and/or under-expressed respectively selected from the group consisting of RPIB9, CXCL2, AL137761, TFPI2, THC2051204, TNF, ABCB1, PURG, ADAMTS5, MCTP1, SPTLC2L, OAS3, MCTP1, GAS1, BIRC3, BQ186674, MAL, UBXD3, and WNT2B; RPIB9, CXCL2, AL137761, TFPI2, TNF, ABCB1, PURG, MCTP1, OAS3, GAS1, BIRC3, BQ186674, and UBXD3; or RPIB9, CXCL2, TFPI2, TNF, ABCB1, PURG, OAS3, GAS1 and BIRC3 for the over-expressed genes; and GALNT14, TM4SF1, ZAR1, A_{—}23_P10091, GLT8D2, RXFP1, CGNL1, AK094972, LRCH2, BM930757, ATP8A1, SOX9, SLC39A8, TMEM47, SLC10A4, SLC1A3, EDG7, ITGA2, PLCXD3, BF514799, SLC16A12, THC2208430, THC2182743, C4orf18, ANKRD38, CALCRL, and LOC152573;GALNT14, TM4SF1, ZAR1, RXFP1, CGNL1, AK094972, SOX9, SLC39A8, TMEM47, SLC10A4, SLC1A3, EDG7, ITGA2, PLCXD3, BF514799, SLC16A12, THC2208430, THC2182743, C4orf18, ANKRD38, and LOC15257; or GALNT14, TM4SF1, RXFP1, SOX9, SLC39A8, SLC1A3, EDG7, ITGA2 and LOC152573/SHISA3 for the under-expressed genes; 2) contacting said cell-line with a test compound; 3) determining the expression level of said at least one gene; and 4) selecting the compound which decreases the expression level of over-expressed genes and increases the expression level of under-expressed genes.
